(19) United States
(12) Patent Application Publication
(10) Pub. No.: US 2026/0022149 A1
(43) Pub. Date: Jan. 22, 2026

(54) APOPTOSIS RESISTANT CELL LINES

(71) Applicant: Genentech, Inc., South San Francisco, CA (US)

(72) Inventors: Shahram MISAGHI, Oakland, CA (US); Danming TANG, Foster City, CA (US); Amy SHEN, San Mateo, CA (US); Michael Wilson LAIRD, Oakland, CA (US)

(73) Assignee: Genentech, Inc., South San Francisco, CA (US)

(21) Appl. No.: 19/349,731

(22) Filed: Oct. 3, 2025

Related U.S. Application Data
(63) Continuation of application No. 17/355,608, filed on Jun. 23, 2021, now abandoned.
(60) Provisional application No. 63/043,545, filed on Jun. 24, 2020.
(60) Provisional application No. 63/210,640, filed on Jun. 15, 2021.

(51) Int. Cl. C07K 14/47; C12N 5/071; C12N 15/67; C12N 15/86

(52) U.S. Cl. CPC C07K 14/4747; C12N 5/0602; C12N 15/67; C12N 15/86; C12N 2510/02; C12N 2511/00

(57) ABSTRACT

The present disclosure relates to eukaryotic cell lines with a stable integrated loss-of-function or attenuation-of-function mutation in each of the Bax and Bak genes. Also provided are methods of producing such cell lines. This disclosure also relates to compositions and cell cultures comprising such cell lines, as well as methods of producing a product, such as a recombinant polypeptide or viral vector, using said cells, compositions and cell cultures.

### CROSS REFERENCE TO RELATED APPLICATIONS

This application claims priority to U.S. Provisional Application No. 63/043,545, filed on Jun. 24, 2020, and to U.S. Provisional Application No. 63/210,640, filed on Jun. 15, 2021, the contents of which are incorporated by reference in its entirety.

### SEQUENCE LISTING

This application contains a Sequence Listing, which has been submitted electronically in XML format and is hereby incorporated by reference in its entirety. Said XML copy, created on Oct. 2, 2025, is named P36177-US-3.xml and is 50,287 bytes in size.

### 1. FIELD OF INVENTION

This disclosure relates to eukaryotic cell lines with a stable integrated loss-of-function or attenuation-of-function mutation in each of the Bax and Bak genes. Also provided are methods of producing such cell lines. This disclosure also relates to compositions and cell cultures comprising said cells, as well as methods of producing a product, such as a recombinant polypeptide or viral vector, and use of said cells, compositions and cell cultures in methods of producing a product of interest.

### 2. BACKGROUND

Monoclonal antibodies (mAbs) and other recombinant proteins have been established as successful therapeutics for many disease indications including immunology, oncology, neuroscience, and others (see, e.g., Reichert (2017) mAbs. 9:167-181; Singh et al. (2017) Curr. Clin. Pharmacol. 13:85-99). With over 300 mAbs in development in the biotechnology industry, the mAb market is projected to expand to 70 mAb products by the year 2020 (Ecker et al. (2015) mAbs. 7:9-14). As the industry expands and targets become more complex, larger antibody discovery campaigns are needed to screen multiple mAb variants and identify clinical candidates with the desired characteristics.

Eukaryotic cells, such as mammalian cells (e.g., Chinese hamster ovary (CHO) cells), have been widely used in the production of therapeutic proteins for clinical applications, such as mAbs, because of their capacity for proper protein folding, assembly, and post translational modifications. Cell culture and production of desired molecules in significant quantities is, however, challenging. It is therefore desirable to provide improved cells and methods for further optimisation of the production of desired products, such as therapeutic proteins).

### 3. SUMMARY

There remains a need for optimal methods for culturing eukaryotic cell lines, such as mammalian cell lines (e.g., CHO cell lines), in order to produce products of interest, such as recombinant polynucleotides or recombinant polypeptides. We have identified that when cell lines are used to produce products of interest, it is advantageous (e.g. provides better product titers) for cells to exhibit high viability. As such, a need exists for cell lines, including mammalian cell lines (e.g. CHO cell lines), with resistance to apoptosis in order to provide higher productivity and more robust performance in bioreactors that their wild type counterparts.

In order to meet these and other demands, provided herein are eukaryotic cell lines, such as mammalian cell lines (e.g., CHO cell lines), with a stable integrated loss-of-function or attenuation-of-function mutation in each of the Bax and Bak genes. The present disclosure accordingly relates to methods, cells, and compositions comprising cells for producing a product of interest, e.g., a recombinant polynucleotide and/or a recombinant polypeptide using the cells of the present disclosure. In particular, the methods, cells and compositions described herein include improved mammalian cells expressing the product of interest, where the cells (e.g., Chinese Hamster Ovary (CHO) cells) have a stable integrated loss-of-function or attenuation-of-function mutation in each of the Bax and Bak genes. The downregulation or deletion of Bax and Bak genes in the cells and cell lines, reduce the undesired effects associated with the undesired apoptotic activity, e.g., reduced viability and productivity of the eukaryotic cells.

A one aspect, the present disclosure provides an isolated eukaryotic cell line, wherein the cell line comprises a stable integrated loss-of-function or attenuation-of-function mutation in each of the Bax and Bak genes.

In certain embodiments, the cell line comprises a stable integrated loss-of-function mutation in each of the Bax and Bak genes.

In certain embodiments, the cell line comprises a deletion in each of the Bax and Bak genes.

In certain embodiments, the cell line is an animal cell line or a fungal cell line. The cell line may be an animal cell line, e.g. a mammalian cell line. Exemplary mammalian cell lines include hybridoma cell lines, CHO cell lines, COS cell lines, VERO cell lines, HeLa cell lines, HEK 293 cell lines, PER-C6 cell lines, K562 cell lines, MOLT-4 cell lines, Ml cell lines, NS-1 cell lines, COS-7 cell lines, MDBK cell lines, MDCK cell lines, MRC-5 cell lines, WI-38 cell lines, WEHI cell lines, SP2/0 cell lines, BHK cell lines (including BHK-21 cell lines), or their derivatives. The cell line may be a CHO cell line, e.g. a CHO K1 cell line, a CHO K1SV cell line, a DG44 cell line, a DUKXB-11 cell line, a CHOK1S cell line, or a CHO K1M cell line, or their derivatives. The cell line may be a fungal cell line, e.g. a yeast cell line.

In certain embodiments, the cell line further comprises a viral genome and one or more polynucleotides encoding a viral capsid.

In certain embodiments, the cell line further comprises a polynucleotide encoding a product of interest.

The polynucleotide that encodes the product of interest may be integrated in the cellular genome of the cell line at a targeted location. The polynucleotide that encodes the product of interest may be randomly integrated in the cellular genome of the cell line. The polynucleotide that encodes the product of interest may be an extrachromosomal polynucleotide. The polynucleotide that encodes the product of interest may be integrated into a chromosome of the cell line.

The product of interest may be or comprise a recombinant polypeptide. The product of interest (such as a recombinant polypeptide) may be or comprise an antibody, an antibody-fusion protein, an antigen, an enzyme, or a vaccine. The product of interest may be or comprise an antibody. The product of interest may be or comprise an antigen. The product of interest may be or comprise an enzyme. The product of interest may be or comprise a vaccine.

The antibody may be a multispecific antibody or antigen-binding fragment thereof. The antibody may be a multispecific antibody or antigen-binding fragment thereof. The antibody may consist of a single heavy chain sequence and a single light chain sequence or antigen-binding fragments thereof. The antibody may comprise a chimeric antibody, a human antibody or a humanized antibody. The antibody may comprise a monoclonal antibody.

In certain embodiments, the cell line has a higher specific productivity than a corresponding isolated eukaryotic cell line that comprises the polynucleotide and functional copies of each of the wild type Bax and Bak genes.

In certain embodiments, the cell line is more resistant to apoptosis than a corresponding isolated eukaryotic cell line that comprises functional copies of each of the Bax and Bak genes.

In certain embodiments, the cell line is employed in cell culture processes such as fed-batch, perfusion, process intensified perfusion, semi-continuous perfusion, or continuous perfusion. For example, but not limitation, the cell line may be employed in an intensified perfusion process.

In another aspect, the present disclosure provides a composition comprising a eukaryotic cell line of the invention, for example a cell line of the first aspect. The composition may also comprise a cell culture medium.

In certain embodiments, the composition is employed in a cell culture process, such as fed-batch, perfusion, process intensified, semi-continuous perfusion, or continuous perfusion. For example, but not limitation, the cell culture process may be an intensified perfusion process.

In another aspect, the present disclosure provides a cell culture comprising a cell culture medium and a plurality of eukaryotic cells, wherein each cell of the plurality comprises a stable integrated loss-of-function or attenuation-of-function mutation in each of the Bax and Bak genes.

In certain embodiments, the cell culture is employed in cell culture processes such as fed-batch, perfusion, process intensified, semi-continuous perfusion, or continuous perfusion. The cell culture process may be an intensified perfusion process.

In certain embodiments, each cell comprises a stable integrated loss-of-function mutation in each of the Bax and Bak genes.

In certain embodiments, each cell of the plurality comprises a deletion in each of the Bax and Bak genes.

In certain embodiments, the cells are animal cells or fungal cells. The cells may be animal cells, e.g. mammalian cells. Exemplary mammalian cells include hybridoma cells, CHO cells, COS cells, VERO cells, HeLa cells, HEK 293 cells, PER-C6 cells, K562 cells, MOLT-4 cells, Ml cells, NS-1 cells, COS-7 cells, MDBK cells, MDCK cells, MRC-5 cells, WI-38 cells, WEHI cells, SP2/0 cells, BHK cells (including BHK-21 cells), or their derivatives. The cells may be CHO cells, e.g. CHO K1 cells, CHO K1SV cells, DG44 cells, DUKXB-11 cells, CHOK1S cells, or CHO K1M cells, or their derivatives. The cells may be fungal cells, e.g. yeast cells.

In certain embodiments, the cells further comprise a viral genome and one or more polynucleotides encoding a viral capsid.

In certain embodiments, the cell culture (e.g. the plurality of cells) further comprises a polynucleotide that encodes a product of interest.

In certain embodiments, the polynucleotide that encodes the product of interest may be integrated in the cellular genome of the cells at a targeted location. In certain embodiments, the polynucleotide that encodes the product of interest may be randomly integrated in the cellular genome of the cells. In certain embodiments, the polynucleotide that encodes the product of interest may be an extrachromosomal polynucleotide. In certain embodiments, the polynucleotide that encodes the product of interest may be integrated into a chromosome of the cells.

In certain embodiments, the product of interest may be or comprise a recombinant polypeptide. In certain embodiments, the product of interest (such as a recombinant polypeptide) may be or comprise an antibody, an antibody-fusion protein, an antigen, an enzyme, or a vaccine. The product of interest may be or comprise an antibody. In certain embodiments, the product of interest may be or comprise an antigen. In certain embodiments, the product of interest may be or comprise an enzyme. In certain embodiments, the product of interest may be or comprise a vaccine.

In certain embodiments, the antibody may be a multispecific antibody or antigen-binding fragment thereof. In certain embodiments, the antibody may be a multispecific antibody or antigen-binding fragment thereof. In certain embodiments, the antibody may consist of a single heavy chain sequence and a single light chain sequence or antigen-binding fragments thereof. In certain embodiments, the antibody may comprise a chimeric antibody, a human antibody or a humanized antibody. The antibody may comprise a monoclonal antibody.

In certain embodiments, each of the cells further comprises a recombinant polynucleotide.

In another aspect, the present disclosure provides a method of reducing apoptotic activity in a eukaryotic cell, comprising administering to the cell a genetic engineering system. In certain embodiments, the genetic engineering system: (a) knocks down or knocks out the expression of a Bax polypeptide isoform; and (b) knocks down or knocks out the expression of a Bak polypeptide isoform.

In certain embodiments, the method further comprises employing the eukaryotic cell in a fed-batch, perfusion, process intensified, semi-continuous perfusion, or continuous perfusion cell culture process. The eukaryotic cell may be employed in an intensified cell culture process.

In certain embodiments, the genetic engineering system is selected from the group consisting of a CRISPR/Cas system (e.g. a CRISPR/Cas9 system), a zinc-finger nuclease (ZFN) system, a transcription activator-like effector nuclease (TALEN) system and a combination thereof. The genetic engineering system may be a CRISPR/Cas system. The genetic engineering system may be a ZFN system. The genetic engineering system may be a TALEN system.

In certain embodiments, the genetic engineering system is or comprises a CRISPR/Cas9 system. The CRISPR/Cas9 system may comprise: (a) a Cas9 molecule, (b) at least one first guide RNA (gRNA) comprising a targeting sequence that is complementary to a target sequence in a Bax gene, and (c) at least one second gRNA comprising a targeting sequence that is complementary to a target sequence in a Bak gene. At least one of the target sequences may be a portion of the Bax gene. At least one of the target sequences may be a portion of the Bak gene. At least one of the target sequences may be a portion of the Bax gene, and at least one other of the target sequences may be a portion of the Bak gene.

In certain embodiments, the expression of the Bax polypeptide or the expression of the Bak polypeptide is knocked out, and the apoptotic activity of the cell is reduced compared to the apoptotic activity of a reference cell. In an embodiment, the expression of the Bax polypeptide and the expression of the Bak polypeptide is knocked out, and the apoptotic activity of the cell is reduced compared to the apoptotic activity of a reference cell.

In certain embodiments, the expression of the Bax polypeptide or the expression of the Bak polypeptide is knocked down, and the apoptotic activity of the cell is reduced compared to the apoptotic activity of a reference cell. In an embodiment, the expression of the Bax polypeptide or the expression of the Bak polypeptide is knocked down, and the apoptotic activity of the cell is reduced compared to the apoptotic activity of a reference cell.

In certain embodiments, the apoptotic activity is less than the apoptotic activity in the reference cell (e.g. the apoptotic activity may be less than about 80%, less than about 50%, or less than about 30% of the apoptotic activity in the reference cell). For example, the apoptotic activity may be less than from about 1% to less than about 99% apoptotic activity of the reference cell. The apoptotic activity of the cell may be determined from the viability for a population of said cells compared to the viability of a population of said reference cells determined during production phase. The reference cell may be a cell that comprises wild-type alleles of the Bax and Bak genes, for example the reference cell may be a cell that only substantially differs from the apoptosis attenuated cell in that the reference cell comprises wild-type alleles of the Bax and Bak genes. In an embodiment, less apoptosis correlates with cells that possess high viability.

In certain embodiments, the cell is an animal cell or a fungal cell. The cell may be an animal cell, e.g. a mammalian cell. Exemplary mammalian cells include hybridoma cells, CHO cells, COS cells, VERO cells, HeLa cells, HEK 293 cells, PER-C6 cells, K562 cells, MOLT-4 cells, Ml cells, NS-1 cells, COS-7 cells, MDBK cells, MDCK cells, MRC-5 cells, WI-38 cells, WEHI cells, SP2/0 cells, BHK cells (including BHK-21 cells), or their derivatives. The cell may be a CHO cell, e.g. a CHO K1 cell, a CHO K1SV cell, a DG44 cell, a DUKXB-11 cell, a CHOK1S cell, or a CHO K1M cell, or their derivatives. The cell may be a fungal cell, e.g. a yeast cell.

In certain embodiments, the cell further comprises a viral genome and one or more polynucleotides encoding a viral capsid.

In certain embodiments, the cell further comprises a polynucleotide that encodes a product of interest.

In certain embodiments, the polynucleotide that encodes the product of interest may be integrated in the cellular genome of the cell at a targeted location. In certain embodiments, the polynucleotide that encodes the product of interest may be randomly integrated in the cellular genome of the cell. In certain embodiments, the polynucleotide that encodes the product of interest may be an extrachromosomal polynucleotide. In certain embodiments, the polynucleotide that encodes the product of interest may be integrated into a chromosome of the cell.

In certain embodiments, the product of interest may be or comprise a recombinant polypeptide. The product of interest (such as a recombinant polypeptide) may be or comprise an antibody, an antibody-fusion protein, an antigen, an enzyme, or a vaccine. In certain embodiments, the product of interest may be or comprise an antibody. In certain embodiments, the product of interest may be or comprise an antigen. In certain embodiments, the product of interest may be or comprise an enzyme. In certain embodiments, the product of interest may be or comprise a vaccine.

In certain embodiments, the antibody may be a multispecific antibody or antigen-binding fragment thereof. In certain embodiments, the antibody may be a multispecific antibody or antigen-binding fragment thereof. In certain embodiments, the antibody may consist of a single heavy chain sequence and a single light chain sequence or antigen-binding fragments thereof. In certain embodiments, the antibody may comprise a chimeric antibody, a human antibody or a humanized antibody. In certain embodiments, the antibody may comprise a monoclonal antibody.

In certain embodiments, each of the cells further comprises a recombinant polynucleotide.

In another aspect, the present disclosure provides a method of producing a recombinant polypeptide. In certain embodiments, the method comprises culturing a eukaryotic cell line, under conditions suitable for production of the polypeptide. In certain embodiments, the cell line comprises (a) a stable integrated loss-of-function or attenuation-of-function mutation in each of the Bax and Bak genes, and (b) a polynucleotide encoding the recombinant polypeptide.

In certain embodiments, polynucleotide that encodes the polypeptide is integrated in the cellular genome of the cells of the cell line at a targeted location. In certain embodiments, the polynucleotide that encodes the polypeptide is randomly integrated in the cellular genome of the cells of the cell line.

In certain embodiments, the polynucleotide that encodes the polypeptide is an extrachromosomal polynucleotide. In certain embodiments, the polynucleotide that encodes the polypeptide is integrated into a chromosome of the cells of the cell line.

In certain embodiments, the recombinant polypeptide may be or comprise an antibody, an antigen, an enzyme, or a vaccine. In certain embodiments, the recombinant polypeptide may be or comprise an antibody. In certain embodiments, the recombinant polypeptide may be or comprise an antibody-fusion protein. In certain embodiments, the recombinant polypeptide may be or comprise an antigen. In certain embodiments, the recombinant polypeptide may be or comprise an enzyme. The recombinant polypeptide may be or comprise a vaccine.

In certain embodiments, the antibody may be a multispecific antibody or antigen-binding fragment thereof. In certain embodiments, the antibody may be a multispecific antibody or antigen-binding fragment thereof. In certain embodiments, the antibody may consist of a single heavy chain sequence and a single light chain sequence or antigen-binding fragments thereof. In certain embodiments, the antibody may comprise a chimeric antibody, a human antibody or a humanized antibody. In certain embodiments, the antibody may comprise a monoclonal antibody.

In certain embodiments, the method further comprises isolating the recombinant polypeptide. The isolating typically comprises isolating the recombinant polypeptide from the cell line.

In certain embodiments, the cell line is an animal cell line or a fungal cell line. The cell line may be an animal cell line, e.g. a mammalian cell line. Exemplary mammalian cell lines include hybridoma cell lines, CHO cell lines, COS cell lines, VERO cell lines, HeLa cell lines, HEK 293 cell lines, PER-C6 cell lines, K562 cell lines, MOLT-4 cell lines, Ml cell lines, NS-1 cell lines, COS-7 cell lines, MDBK cell lines, MDCK cell lines, MRC-5 cell lines, WI-38 cell lines, WEHI cell lines, SP2/0 cell lines, BHK cell lines (including BHK-21 cell lines), or their derivatives. The cell line may be a CHO cell line, e.g. a CHO K1 cell line, a CHO K1SV cell line, a DG44 cell line, a DUKXB-11 cell line, a CHOK1S cell line, or a CHO K1M cell line, or their derivatives. The cell line may be a fungal cell line, e.g. a yeast cell line.

In certain embodiments, the cell line is cultured in a cell culture medium. The cell line may be cultured under fed-batch culture conditions, or perfusion culture conditions. The cell line may be cultured under fed-batch culture conditions. The fed-batch culture conditions may be intensified fed-batch culture conditions. The cell line may be cultured under perfusion culture conditions. The perfusion culture conditions may be semi-continuous perfusion. The perfusion culture conditions may be continuous perfusion.

In certain embodiments, the cell line comprises a stable integrated loss-of-function mutation in each of the Bax and Bak genes.

In another aspect, the present disclosure provides a method of producing a viral vector. In certain embodiments, the method comprises culturing a eukaryotic cell line, under conditions suitable for production of the viral vector. In certain embodiments, the cell line comprises (a) stable integrated a loss-of-function or attenuation-of function mutation in each of the Bax and Bak genes, (b) a viral genome, and (c) one or more polynucleotides encoding a viral capsid, under conditions suitable for production of the viral vector.

In certain embodiments, the method further comprising isolating the viral vector. The isolating typically comprises isolating the viral vector from the cell line.

In certain embodiments, the cell line is an animal cell line or a fungal cell line. The cell line may be an animal cell line, e.g., a mammalian cell line. Exemplary mammalian cell lines include hybridoma cell lines, CHO cell lines, COS cell lines, VERO cell lines, HeLa cell lines, HEK 293 cell lines, PER-C6 cell lines, K562 cell lines, MOLT-4 cell lines, Ml cell lines, NS-1 cell lines, COS-7 cell lines, MDBK cell lines, MDCK cell lines, MRC-5 cell lines, WI-38 cell lines, WEHI cell lines, SP2/0 cell lines, BHK cell lines (including BHK-21 cell lines), or their derivatives. The cell line may be a CHO cell line, e.g. a CHO K1 cell line, a CHO K1SV cell line, a DG44 cell line, a DUKXB-11 cell line, a CHOK1S cell line, or a CHO K1M cell line, or their derivatives. The cell line may be a fungal cell line, e.g. a yeast cell line.

In certain embodiments, the cell line is cultured in a cell culture medium. The cell line may be cultured under fed-batch culture conditions, or perfusion culture conditions. The cell line may be cultured under fed-batch culture conditions. The fed-batch culture conditions may be intensified fed-batch culture conditions. The cell line may be cultured under perfusion culture conditions. The perfusion culture conditions may be semi-continuous perfusion. The perfusion culture conditions may be continuous perfusion.

In certain embodiments, the cell line comprises a stable integrated loss-of-function mutation in each of the Bax and Bak genes.

In another aspect, the present disclosure provides a method of producing a recombinant polypeptide, comprising a method of reducing apoptotic activity according to the fourth aspect, followed by producing the recombinant polypeptide according to a method of the fifth aspect.

In another aspect, the present disclosure provides a method of producing a viral vector, comprising a method of reducing apoptotic activity according to the fourth aspect, followed by producing the viral vector according to a method of the sixth aspect.

In another aspect, the present disclosure provides use of an isolated eukaryotic cell line of the first aspect for the production of a product of interest, the cell line comprising a polynucleotide encoding the product of interest. The use may further comprise isolating the product of interest.

In another aspect, the present disclosure provides use of a composition of the second aspect for the production of a product of interest, wherein the cell line of said composition comprises a polynucleotide encoding the product of interest. The use may further comprise isolating the product of interest.

In another aspect, the present disclosure provides use of a cell culture of the third aspect for the production of a product of interest, wherein the plurality of eukaryotic cells of said cell culture further comprises a polynucleotide that encodes a product of interest. The use may further comprise isolating the product of interest.

In another aspect, the present disclosure provides use of a cell-line of the first aspect, composition of the second aspect, or cell culture of the third aspect in a cell culture process. The cell culture process may be or comprise a fed-batch, perfusion, process intensified, semi-continuous perfusion, or continuous perfusion cell culture process. The cell culture process may comprise an intensified perfusion process.

### 4. BRIEF DESCRIPTION OF THE DRAWINGS

FIG. 1 provides the day 14 IVCC results for 4 to 5 clones with highest antibody titers, generated from wild type (WT) and two different Bax/Bak double knockout (DKO) hosts, respectively, were analyzed for their integral of viable cell concentration (IVCC, 1e8 cell-d/L) over the 14 day period of intensified (high seeding density) antibody production process. Bax/Bak DKO clones had comparable or higher IVCC than the WT clones.

FIG. 2 provides the VCC of WT and Bax/Bak DKO clones during the intensified production process. Viable cell count (VCC, 1e6 cell/mL) of the indicated clones from the WT host (A) or two different Bax/Bak DKO hosts (B&C) were measured and plotted. Bax/Bak DKO clones and WT clones had similar growth rate in the first 2 days. VCCs declined after day 3 because the cell cultures were diluted everyday by adding feed and removing cultured cells for various assays.

FIG. 3 provides the Viability of WT and Bax/Bak DKO clones during the intensified production process. Viability (%) of the indicated clones generated from the WT host (A) or two different Bax/Bak DKO hosts (B&C) were measured and plotted. WT clones had declined viabilities after day 10 (A), while Bax/Bak DKO clones maintained high viability till the end of the process, suggesting that deletion of Bax and Bak genes significantly prevents cell death in the later stage of the intensified process.

FIG. 4 provides the day 14 viability (%) of indicated clones. Bax/Bak DKO clones showed much higher viability than the WT clones on day 14, confirming that deletion of Bax and Bak genes significantly reduces cell death in the later stage of the intensified process.

FIG. 5 provides a Western blot analysis of cleaved caspase-3 in Day −14 cell pellets. Day 14 cell pellets of the indicated clones were analyzed by Western blot for the levels of cleaved caspase-3, an apoptosis marker protein. All the WT clones expressed high levels of cleaved caspase-3, indicating that WT cells are undergoing apoptosis in the later stages of the intensified process, and that apoptotic cell death is a major contributor to culture viability decline. All the Bax/Bak DKO clones had low levels of cleaved caspase-3 cleavage, suggesting that deletion of Bax and Bak genes sufficiently blocks apoptotic cell death.

FIG. 6 illustrates the titres obtained on on days 3, 7, 10 and 14. Antibody titers (g/L) on days 3, 7, 10 and 14 in a 14-day intensified process for indicated clones were measured and plotted. Note that Bax/Bak DKO clones day 7 titers were on average comparable to the WT clones, while their day 14 titers were significantly higher. More importantly, for most of the Bax/Bak DKO clones, day 14 titers were higher than day 10 titer, indicating that cells were still producing antibody in the last 4 days of production culture. However for the WT clones, titers did not increase from day 10 to day 14, suggesting that these clones lost productivity at the end of the intensified production process. The loss of productivity in the WT clones was likely due to apoptotic cell death in these cultures.

FIG. 7 indicates the average specific productivity. Cell specific productivities (Qp, pg/cell-d) were calculated from dividing day 14 titer (g/L) by day 14 IVCC (1e8 cell-d/L). Qp of Bax/Bak DKO clones were on average higher than that of the WT clones.

FIG. 8 indicates the corrected average specific productivities for the top clones in the whole 14-day process. Cell specific productivities (Qp, pg/cell-d) corrected by dilution factors. Results are provided in FIG. 8 for the top clone of WT host and the top 2-3 clones from Bax/Bak DKO hosts.

FIG. 9 provides the corrected specific productivities of the top clones at different stages during the intensified production process. Corrected specific productivities (Qp, pg/cell-d) of the indicated top clones are provided at different stages during the intensified process. For all clones, day 0 to 3 was the cell growth stage, when Qp was lower than it was during the stationary stage (after day 3). Only the WT clone showed a declined Qp between Day 10 to 14. Since Qp was calculated only for the viable cells, this result suggests that WT cells not only declined in viability but also in productivity in the last 4 days of the process. This decline of productivity is likely due to mitochondrial membrane damage caused by Bax and Bak proteins activation at the onset of apoptosis. On the other hand, the Bax/Bak DKO clones not only had extended viability but also had extended productivity. Accordingly, it is considered that the deletion of these two genes not only prevented apoptosis, but it also assisted in maintaining mitochondrial integrity and health.

FIG. 10 illustrates the glucose consumption rates for the top clones. Total glucose consumed during the intensified production process (mg) was plotted against the integral of total cell number (1e6cell-d) at different time points. The slopes represent glucose consumption rates (mg/1e6 cell-d) for indicated clones. The glucose consumption rates were comparable between Bax/Bak DKO clones and WT clone.

FIG. 11 indicates the culture lactate concentrations of the top clones during the intensified production process. Lactate concentration in the harvested cell culture fluid (HCCF) of the indicated clones were measured daily and plotted. The results suggest that lactate metabolism of WT and Bax/Bak DKO clones were comparable during the intensified production process.

FIG. 12 provides the day 14 HMWS (%). The levels of aggregated antibodies (%) in day 14 HCCF are given in FIG. 14 for the indicated clones. The % HMWS levels were on average comparable between the WT and Bax/Bak DKO clones.

FIG. 13 indicates the day 14% Main peak. This provides an illustration of the levels of the intact and monomeric antibodies (%) in day 14 HCCF for the indicated clones. The % Main peak levels were on average comparable between the WT and Bax/Bak DKO clones

FIG. 14 provides an illustration of the amount of antibody fragments as % LMWS. Levels of antibody fragments in day 14 HCCF for the indicated clones are depicted. The % LMWS levels were on average comparable between the WT and Bax/Bak DKO clones.

FIG. 15 depicts the amounts of antibody acidic charge variants (%) in day 14 HCCF of the clones. The % Acidic peak levels were on average comparable between the WT and Bax/Bak DKO clones.

FIG. 16 provides the results for main peaks, which indicates the levels of antibody neutral charge variants (%) in day 14 HCCF of the clones. The % Main peak levels were on average comparable between the WT and Bax/Bak DKO clones.

FIG. 17 depicts the amounts of antibody basic charge variants (%) in day 14 HCCF of the clones. The % Basic peak levels were on average comparable between the WT and Bax/Bak DKO clones.

FIGS. 18A-18C illustrate that knocking out Bax/Bak genes improve cell viability and titer for standard mAb expressing CHO pools in an intensified production process. Viability, viable cell count (VCC), titer, and 14-day average specific productivity (Qp) of pools of cells expressing mAb-A that were generated from the indicated host cell lines have been measured in a low seeding density platform-1 process in shake flasks (18A), in a low seeding density platform-1 process in AMBR15 bioreactors (18B), and in a high seeding density platform-1 process in AMBR15 bioreactors (18C).

FIGS. 19A-B illustrate that single cell clones generated from Bax/Bak DKO hosts achieved extended viability and higher titer of standard mAb in an intensified production process. Viability, VCC, titer, and 14-day average specific productivity of top clones expressing mAb-A generated from the indicated host cell lines have been measured in (19A) a low seeding density process in shake flasks, and (19B) in an intensified process in AMBR15 bioreactors. Error bars show the standard deviation of 4-5 top clones generated from indicated hosts.

FIG. 20 illustrates that single cell clones generated from Bax/Bak DKO hosts achieved extended viability and higher titer of standard mAb in a scaled-up intensified production process. Viability, VCC, titer, and 14-day average specific productivity of the top clones expressing mAb-A generated from indicated host cell lines have been measured in the intensified process using AMBR250 bioreactors. Error bars show the standard deviation of 4 replicates of the same WT top clone.

FIGS. 21A-21C illustrate that knocking out Bax/Bak genes improves complex molecule expression in a CHO intensified production process. Viability, VCC, titer, and 14-day average specific productivity of the pools of cells expressing complex molecule-B (21A), bispecific antibody molecule-C (21B) and complex molecule-D (21C) generated from WT and two DKO host cell lines have been measured in the intensified production process in AMBR15 bioreactors. Error bars show the standard deviation of 2 replicate pools that were derived from the same host for indicated molecules.

FIGS. 22A-22D illustrate the generation of Bax/Bak DKO cell lines and product quality attributes of mAb-A produced in WT and DKO pools in the 3 production processes. Sequential knock-out of Bax and Bak genes from WT cell line were performed as follows. Step 1, transfecting RNA targeting Bax gene into WT cells followed by single cell cloning to generate Bax KO clone #40. Step 2, transfecting RNA targeting Bak gene into Bax KO clone #40 cells followed by single cell cloning to generate Bax/Bak DKO clone 1, 2, 3, 7, 8, 21. (22B-22D). High molecular weight species (HMWS)/aggregate levels, different glycan species levels, and charge variant levels were measured for mAb-A expressing pools production platform-1 in shake flasks at low seeding density (SD) (22B), in production platform-1 in AMBR15 at low SD (22C), and in production platform-1 in AMBR15 high SD (22D).

FIGS. 23A-23C depict product quality attributes of mAb-A expressing top clones generated from WT and DKO hosts in shake flasks and AMBR15 bioreactors. Different glycan species levels, charge variant levels, and high molecular weight species (HMWS)/aggregate levels for mAb-A expressing clones were measured in production platform-1 in (23A) shake flasks at low SD, and (23B) in AMBR15 intensified production platform-1. Western blot of cleaved caspase 3 levels in Day 14 cells in AMBR15 intensified production platform-1 (23C).

FIG. 24A-24C depict product quality attributes of complex molecules and a bispecific antibody expressed in CHO pools in the intensified process in AMBR15 bioreactors. Charge variant levels, high molecular weight species (HMWS)/aggregate levels, and different glycan species levels were measured for CHO pools expressing complex molecule-B (24A), bispecific molecule-C (24B), and complex molecule C (24C). Note that bispecific molecule-C is an aglycosylated molecule, therefore no glycosylation data for this molecule is available.

FIG. 25A-25D illustrate that knocking out Bax/Bak genes in a pool of transfected CHO cells expressing complex molecule-E improved cell viability in the high seeding density production process. (25A) Overview of strategy to evaluate Bax/Bak gene knockout in pool of CHO cells transfected with complex molecule-E expressing constructs. Titer (25B), cell viability (25C) and VCC (25D) of complex molecule-E expressing pool of CHO cells after mock or Bax/Bak gRNA transfection.

### 5. DETAILED DESCRIPTION

Throughout the description and claims of this specification, the words “comprise” and “contain” and variations of them mean “including but not limited to”, and they are not intended to (and do not) exclude other moieties, additives, components, integers or steps. Throughout the description and claims of this specification, the singular encompasses the plural unless the context otherwise requires. In particular, where the indefinite article is used, the specification is to be understood as contemplating plurality as well as singularity, unless the context requires otherwise.

Features, integers, characteristics, compounds, chemical moieties or groups described in conjunction with a particular aspect, embodiment or example of the invention are to be understood to be applicable to any other aspect, embodiment or example described herein unless incompatible therewith. All of the features disclosed in this specification (including any accompanying claims, abstract and drawings), and/or all of the steps of any method or process so disclosed, may be combined in any combination, except combinations where at least some of such features and/or steps are mutually exclusive. The invention is not restricted to the details of any disclosed embodiments. The invention extends to any novel one, or any novel combination, of the features disclosed in this specification (including any accompanying claims, abstract and drawings), or to any novel one, or any novel combination, of the steps of any method or process so disclosed.

The reader's attention is directed to all papers and documents which are filed concurrently with or previous to this specification in connection with this application and which are open to public inspection with this specification, and the contents of all such papers and documents are incorporated herein by reference.

All references cited herein, including patent applications, patent publications, non-patent literature and UniProtKB/Swiss-Prot Accession numbers are herein incorporated by reference in their entirety, as if each individual reference were specifically and individually indicated to be incorporated by reference.

For the avoidance of doubt, it is hereby stated that the information disclosed earlier in this specification under the heading “Background” is relevant to the invention and is to be read as part of the disclosure of the invention.

For clarity, but not by way of limitation, the detailed description of the presently disclosed subject matter is divided into the following subsections:
5.1 Definitions;
5.2 Methods for Modulating BAX and BAK Expression
5.3 Cells Lines;
5.4 Cell Cultures;
5.5 Methods of Production; and
5.6 Products.

### 5.1. Definitions

The following explanations of terms and methods are provided to better describe the present disclosure and to guide those of ordinary skill in the art in the practice of the present disclosure.

As used herein, the term “about” or “approximately” means within an acceptable error range for the particular value as determined by one of ordinary skill in the art, which will depend in part on how the value is measured or determined, i.e., the limitations of the measurement system. For example, “about” can mean within 3 or more than 3 standard deviations, per the practice in the art. Alternatively, “about” can mean a range of up to 20%, preferably up to 10%, more preferably up to 5%, and more preferably still up to 1% of a given value. Alternatively, particularly with respect to biological systems or processes, the term can mean within an order of magnitude, preferably within 5-fold, and more preferably within 2-fold, of a value.

As used herein, “polypeptide” and “protein” can be used interchangeably and refer generally to peptides and proteins having more than about 10 covalently attached amino acids linked by a peptidyl bond. The term protein encompasses purified natural products, or products which may be produced partially or wholly using recombinant or synthetic techniques. The terms peptide and protein may refer to an aggregate of a protein such as a dimer or other multimer, a fusion protein, a protein variant, or derivative thereof. The term also includes modifications of the protein, for example, protein modified by glycosylation, acetylation, phosphorylation, pegylation, ubiquitination, and so forth. A protein may comprise amino acids not encoded by a nucleic acid codon. A protein may have a sequence of amino acids of sufficient length to produce higher levels of tertiary and/or quaternary structure. A typical protein herein may have a molecular weight of at least about 15-20 kD, preferably at least about 20 kD. Examples of proteins encompassed within the definition herein include all mammalian proteins, in particular, therapeutic and diagnostic proteins, such as therapeutic and diagnostic antibodies, and, in general proteins that contain one or more disulfide bonds, including multi-chain polypeptides comprising one or more inter- and/or intrachain disulfide bonds.

By “protein modification” or “protein mutation” is meant an amino acid substitution, insertion, and/or deletion in a polypeptide sequence or an alteration to a moiety chemically linked to a protein. For example, a modification may be an altered carbohydrate or PEG structure attached to a protein. The proteins of the invention may include at least one such protein modification.

The term “modified protein” or “mutated protein” encompasses proteins having at least one substitution, insertion, and/or deletion of an amino acid. A modified or mutated protein may have 1, 2, 3, 4, 5, 6, 7, 8, 9 or 10 or more amino acid modifications (selected from substitutions, insertions, deletions and combinations thereof.

The term “antibody” as used herein encompasses various antibody structures including, but not limited to, monoclonal antibodies, polyclonal antibodies, monospecific antibodies (e.g., antibodies consisting of a single heavy chain sequence and a single light chain sequence, including multimers of such pairings), multispecific antibodies (e.g., bispecific antibodies) and antibody fragments so long as they exhibit the desired antigen-binding activity.

The terms “antibody fragment”, “antigen-binding portion” of an antibody (or simply “antibody portion”) or “antigen-binding fragment” of an antibody, as used herein, refers to a molecule other than an intact antibody that comprises a portion of an intact antibody that binds the antigen to which the intact antibody binds. Examples of antibody fragments include, but are not limited to, Fv, Fab, Fab′, Fab′-SH, F(ab′)2; diabodies; linear antibodies; single-chain antibody molecules (e.g., scFv, and scFab); single domain antibodies (dAbs); and multispecific antibodies formed from antibody fragments. For a review of certain antibody fragments, see Holliger and Hudson, Nature Biotechnology 23:1126-1136 (2005).

The term “chimeric” antibody means an antibody in which a portion of the heavy and/or light chain is derived from a particular source or species, while the remainder of the heavy and/or light chain is derived from a different source or species.

The term “human antibody” means an antibody which possesses an amino acid sequence which corresponds to that of an antibody produced by a human or a human cell or derived from a non-human source that utilizes human antibody repertoires or other human antibody-encoding sequences. This definition of a human antibody specifically excludes a humanized antibody comprising non-human antigen-binding residues.

The term “humanized antibody” means a chimeric antibody comprising amino acid residues from non-human CDRs and amino acid residues from human FRs. In examples, a humanized antibody will comprise substantially all of at least one, and typically two, variable domains, in which all or substantially all of the CDRs correspond to those of a non-human antibody, and all or substantially all of the FRs correspond to those of a human antibody. A humanized antibody optionally can comprise at least a portion of an antibody constant region derived from a human antibody. A “humanized form” of an antibody, e.g., a non-human antibody, refers to an antibody that has undergone humanization.

The term “monoclonal antibody” means an antibody obtained from a population of substantially homogeneous antibodies, i.e., the individual antibodies comprising the population are identical and/or bind the same epitope, except for possible variant antibodies, e.g., containing naturally occurring mutations or arising during production of a monoclonal antibody preparation, such variants generally being present in minor amounts. In contrast to polyclonal antibody preparations, which typically include different antibodies directed against different determinants (epitopes), each monoclonal antibody of a monoclonal antibody preparation is directed against a single determinant on an antigen. Thus, the modifier “monoclonal” indicates the character of the antibody as being obtained from a substantially homogeneous population of antibodies, and is not to be construed as requiring production of the antibody by any particular method. For example, the monoclonal antibodies in accordance with the presently disclosed subject matter can be made by a variety of techniques, including but not limited to the hybridoma method, recombinant DNA methods, phage-display methods, and methods utilizing transgenic animals containing all or part of the human immunoglobulin loci, such methods and other exemplary methods for making monoclonal antibodies being described herein.

The term “variable region” or “variable domain” means the domain of an antibody heavy or light chain that is involved in binding the antibody to antigen. The variable domains of the heavy chain and light chain (VH and VL, respectively) of a native antibody generally have similar structures, with each domain comprising four conserved framework regions (FRs) and three complementary determining regions (CDRs). (See, e.g., Kindt et al. Kuby Immunology, 6th ed., W.H. Freeman and Co., page 91 (2007).) A single VH or VL domain can be sufficient to confer antigen-binding specificity. Furthermore, antibodies that bind a particular antigen can be isolated using a VH or VL domain from an antibody that binds the antigen to screen a library of complementary VL or VH domains, respectively. See, e.g., Portolano et al., J. Immunol. 150:880-887 (1993); Clarkson et al., Nature 352:624-628 (1991).

The term “hypervariable region” or “HVR” as used herein refers to each of the regions of an antibody variable domain which are hypervariable in sequence and which determine antigen binding specificity, for example “complementarity determining regions” (“CDRs”). Generally, antibodies comprise six CDRs: three in the VH (CDR-H1, CDR-H2, CDR-H3), and three in the VL (CDR-L1, CDR-L2, CDR-L3). Exemplary CDRs herein include:
hypervariable loops occurring at amino acid residues 26-32 (L1), 50-52 (L2), 91-96 (L3), 26-32 (H1), 53-55 (H2), and 96-101 (H3) (Chothia and Lesk, J. Mol. Biol. 196:901-917 (1987));
CDRs occurring at amino acid residues 24-34 (L1), 50-56 (L2), 89-97 (L3), 31-35b (H1), 50-65 (H2), and 95-102 (H3) (Kabat et al., Sequences of Proteins of Immunological Interest, 5th Ed. Public Health Service, National Institutes of Health, Bethesda, MD (1991)); and
antigen contacts occurring at amino acid residues 27c-36 (L1), 46-55 (L2), 89-96 (L3), 30-35b (H1), 47-58 (H2), and 93-101 (H3) (MacCallum et al. J. Mol. Biol. 262: 732-745 (1996)).

CDRs may be determined according to Kabat et al., Sequences of Proteins of Immunological Interest, 5th Ed. Public Health Service, National Institutes of Health, Bethesda, MD (1991). CDR designations may also be determined according to Chothia and Lesk, J. Mol. Biol. 196:901-917 (1987), MacCallum et al. J. Mol. Biol. 262: 732-745 (1996), or any other scientifically accepted nomenclature system.

The term “class” in relation to an antibody means the type of constant domain or constant region possessed by its heavy chain. There are five major classes of antibodies: IgA, IgD, IgE, IgG and IgM, and several of these can be further divided into subclasses (isotypes), e.g., IgG1, IgG2, IgG3, IgG4, IgA1, and IgA2. The antibody may be of the IgG1 isotype. The antibody may be of the IgG2 isotype. The heavy chain constant domains that correspond to the different classes of immunoglobulins are called α, δ, ε, γ and μ, respectively. The light chain of an antibody can be assigned to one of two types, called kappa (κ) and lambda (λ), based on the amino acid sequence of its constant domain.

The terms “nucleic acid molecule” or “polynucleotide” means any compound and/or substance that comprises a polymer of nucleotides. Each nucleotide is composed of a base, specifically a purine- or pyrimidine base (i.e., cytosine (C), guanine (G), adenine (A), thymine (T) or uracil (U)), a sugar (i.e., deoxyribose or ribose), and a phosphate group. Often, the nucleic acid molecule is described by the sequence of bases, whereby said bases represent the primary structure (linear structure) of a nucleic acid molecule. The sequence of bases is typically represented from 5′ to 3′. Herein, the term nucleic acid molecule encompasses deoxyribonucleic acid (DNA) including, e.g., complementary DNA (cDNA) and genomic DNA, ribonucleic acid (RNA), in particular messenger RNA (mRNA), synthetic forms of DNA or RNA, and mixed polymers comprising two or more of these molecules. The nucleic acid molecule can be linear or circular. In addition, the term nucleic acid molecule includes both, sense and antisense strands, as well as single stranded and double stranded forms. Moreover, the herein described nucleic acid molecule can contain naturally occurring or non-naturally occurring nucleotides. Examples of non-naturally occurring nucleotides include modified nucleotide bases with derivatized sugars or phosphate backbone linkages or chemically modified residues. Nucleic acid molecules also encompass DNA and RNA molecules which are suitable as a vector for direct expression of an antibody of the disclosure in vitro and/or in vivo, e.g., in a host or patient. Such DNA (e.g., cDNA) or RNA (e.g., mRNA) vectors, can be unmodified or modified. For example, mRNA can be chemically modified to enhance the stability of the RNA vector and/or expression of the encoded molecule so that mRNA can be injected into a subject to generate the antibody in vivo (see, e.g., Stadler et al, Nature Medicine 2017, published online 12 Jun. 2017, doi:10.1038/nm.4356 or EP 2 101 823 B1). The term “vector” means, unless the context requires otherwise, a nucleic acid molecule capable of transporting another nucleic acid to which it has been linked.

The term “isolated” means a biological component (such as a nucleic acid molecule or protein) that has been substantially separated or purified away from other biological components in the cell of the organism in which the component naturally occurs, i.e., other chromosomal and extrachromosomal DNA and RNA, and proteins. Nucleic acids and proteins that have been “isolated” include nucleic acids and proteins purified by standard purification methods. The term also embraces nucleic acids and proteins prepared by recombinant expression in a host cell as well as chemically synthesized nucleic acids, proteins and peptides.

Purified: The term purified does not require absolute purity; rather, it is intended as a relative term. Thus, for example, a purified product is one in which the product is more enriched than the product (e.g. polypeptide or protein) is in its environment within a cell, such that the product is substantially separated from cellular components (nucleic acids, lipids, carbohydrates, and [other] polypeptides) that may accompany it.

In one example, a product of interest of the disclosure (e.g. a polypeptide, such as an antibody) is purified when at least 50% by weight of a sample is composed of the product, for example when at least 60%, 70%, 80%, 85%, 90%, 92%, 95%, 98%, or 99% or more of a sample is composed of the polypeptide. Examples of methods that can be used to purify a polypeptide, include, but are not limited to the methods disclosed in Sambrook et al. (Molecular Cloning: A Laboratory Manual, Cold Spring Harbor, N.Y., 1989, Ch. 17). Protein purity can be determined by, for example, high-pressure liquid chromatography or other conventional methods.

The term “titer” means the total amount of a product of interest (e.g. a recombinant polypeptide, such as an antibody) produced by a cell culture divided by a given amount of medium volume. Titer is typically expressed in units of milligrams of antibody per milliliter or liter of medium (mg/ml or mg/L). In certain embodiments, titer is expressed in grams of antibody per liter of medium (g/L). Titer can be expressed or assessed in terms of a relative measurement, such as a percentage increase in titer as compared obtaining the protein product under different culture conditions.

The term “sequence identity”: the identity between two or more nucleic acid sequences, or two or more amino acid sequences, is expressed in terms of the identity or similarity between the sequences. Sequence identity can be measured in terms of percentage identity; the higher the percentage, the more identical the sequences are. The percentage identity is calculated over the entire length of the sequence. Homologs or orthologs of nucleic acid or amino acid sequences possess a relatively high degree of sequence identity when aligned using standard methods. This homology is more significant when the orthologous proteins or cDNAs are derived from species which are more closely related (e.g., human and mouse sequences), compared to species more distantly related (e.g., human and C. elegans sequences).

The term “cell” as used herein includes reference to a eukaryotic cell. Unless the context requires otherwise, reference to a cell may include reference to the plural (cells). A eukaryotic cell may be an animal cell (e.g. a mammalian cell) or a fungal cell (e.g. a yeast cell). A eukaryotic cell may be a mammalian cell, such as a hybridoma, CHO cell, COS cell, VERO cell, HeLa cell, HEK 293 cell, PER-C6 cell, K562 cell, MOLT-4 cell, Ml cell, NS-1 cell, COS-7 cell, MDBK cell, MDCK cell, MRC-5 cell, WI-38 cell, WEHI cell, SP2/0 cell, BHK cell (including BHK-21 cell) and derivatives thereof. A CHO cell may be, for example, a CHO K1 cell, a CHO K1SV cell, a DG44 cell, a DUKXB-11 cell, a CHOK1S cell, a CHO K1M cell, and derivatives thereof.

The term “cell line” as used herein includes reference to a culture of eukaryotic cells that can be propagated repeatedly. The eukaryotic cells of the cell line may be selected from any cell as defined herein.

The terms “host cell,” “host cell line” and “host cell culture” are used interchangeably herein to refer to cells into which exogenous nucleic acid has been introduced, including the progeny of such cells. Host cells include “transformants” and “transformed cells,” which include the primary transformed cell and progeny derived therefrom without regard to the number of passages. Progeny does not need to be completely identical in nucleic acid content to a parent cell, but can contain mutations. Mutant progeny that have the same function or biological activity as screened or selected for in the originally transformed cell are included herein.

The terms “mammalian host cell” or “mammalian cell” as used herein refer to cells and cell lines derived from mammals that are capable of growth and survival when placed in either monolayer culture or in suspension culture in a medium containing the appropriate nutrients and growth factors. The necessary growth factors for a particular cell line are readily determined empirically without undue experimentation, as described for example in Mammalian Cell Culture (Mather, J. P. ed., Plenum Press, N.Y. 1984), and Barnes and Sato, (1980) Cell, 22:649. Typically, the cells are capable of expressing and secreting large quantities of a particular protein, e.g., glycoprotein, of interest into the culture medium. Examples of suitable mammalian host cells include Chinese hamster ovary cells/-DHFR (CHO, Urlaub and Chasin, Proc. Natl. Acad. Sci. USA, 77:4216 1980); dp12.CHO cells (EP 307,247 published 15 Mar. 1989); CHO-K1 (ATCC, CCL-61); monkey kidney CV1 line transformed by SV40 (COS-7, ATCC CRL 1651); human embryonic kidney line (293 or 293 cells subcloned for growth in suspension culture, Graham et al., J. Gen Virol., 36:59 1977); baby hamster kidney cells (BHK, ATCC CCL 10); mouse sertoli cells (TM4, Mather, Biol. Reprod., 23:243-251 1980); monkey kidney cells (CV1 ATCC CCL 70); African green monkey kidney cells (VERO-76, ATCC CRL-1587); human cervical carcinoma cells (HELA, ATCC CCL 2); canine kidney cells (MDCK, ATCC CCL 34); buffalo rat liver cells (BRL 3A, ATCC CRL 1442); human lung cells (W138, ATCC CCL 75); human liver cells (Hep G2, HB 8065); mouse mammary tumor (MMT 060562, ATCC CCL51); TRI cells (Mather et al., Annals N.Y. Acad. Sci., 383:44-68 1982); MRC 5 cells; FS4 cells; and a human hepatoma line (Hep G2). The mammalian cells may include Chinese hamster ovary cells/-DHFR (CHO, Urlaub and Chasin, Proc. Natl. Acad. Sci. USA, 77:4216 1980); dp12.CHO cells (EP 307,247 published 15 Mar. 1989).

The term “hybridoma” means a hybrid cell line produced by the fusion of an immortal cell line of immunologic origin and an antibody producing cell. The term encompasses progeny of heterohybrid myeloma fusions, which are the result of a fusion with human cells and a murine myeloma cell line subsequently fused with a plasma cell, commonly known as a trioma cell line. Furthermore, the term is meant to include any immortalized hybrid cell line which produces antibodies such as, for example, quadromas. See, e.g., Milstein et al., Nature, 537:3053 (1983).

The term “cell culture medium” as used herein refers to a nutritive solution for cultivating cells. A “cell culture feed” and a “cell culture additive” represent nutritive supplements that may be added to a cell culture medium to improve medium performance. For example, a cell culture feed and/or a cell culture additive may be added to a cell culture medium during batch culture of cells. A cell culture medium may be chemically defined or may comprise undefined components. Cell culture medium, for example for mammalian cells, typically comprises at least one component from one or more of the following categories:
1) an energy source, usually in the form of a carbohydrate such as glucose;
2) all essential amino acids, and usually the basic set of twenty amino acids plus cysteine;
3) vitamins and/or other organic compounds required at low concentrations;
4) free fatty acids; and
5) trace elements, where trace elements are defined as inorganic compounds or naturally occurring elements that are typically required at very low concentrations, usually in the micromolar range.

Cell culture media and similar nutrient solutions may optionally be supplemented with one or more components from any of the following categories:
1) hormones and other growth factors as, for example, insulin, transferrin, and epidermal growth factor;
2) salts and buffers as, for example, calcium, magnesium, and phosphate;
3) nucleosides and bases such as, for example, adenosine, thymidine, and hypoxanthine; and
4) protein and tissue hydrolysates.

A “chemically defined” medium as used herein is a medium in which every ingredient is known. A chemically defined medium is distinguished from serum, embryonic extracts, and hydrolysates, each of which contain unknown components. A cell culture medium of the present disclosure may be a chemically defined medium. A cell culture feed of the present disclosure may be chemically defined. A cell culture additive of the present disclosure may be chemically defined.

An “undefined medium” or “medium comprising undefined component(s)” as used herein includes reference to a medium that comprises one or more ingredients that are not known. Undefined components may be provided by, for example, serum, peptones, hydrolysates (such as yeast, plant or serum hydrolysate), and embryonic extracts.

The term “culturing” refers to contacting a cell or cells with a cell culture medium under conditions suitable to the survival and/or growth and/or proliferation of the cell.

The term “batch culture” refers to a culture in which all components for cell culturing (including the cells and all culture nutrients) are supplied to the culturing bioreactor at the start of the culturing process.

The term “fed batch cell culture,” as used herein refers to a batch culture wherein the cells and culture medium are supplied to the culturing bioreactor initially, and additional culture nutrients are fed, continuously or in discrete increments, to the culture during the culturing process, with or without periodic cell and/or product harvest before termination of culture.”

The term “perfusion culture,” sometimes referred to as continuous culture, is a culture by which the cells are restrained in the culture by, e.g., filtration, encapsulation, anchoring to microcarriers, etc., and the culture medium is continuously, step-wise or intermittently introduced (or any combination of these) and removed from the culturing bioreactor.”

The term “growth phase” of a cell culture refers to the period of exponential cell growth (the log phase) where cells are generally rapidly dividing. The duration of time for which the cells are maintained at growth phase can vary based on the cell-type, the rate of growth of cells and/or the culture conditions, for example. During this phase, cells are cultured for a period of time, for example 1-4 days, and under such conditions that cell growth is maximized. The determination of the growth cycle for the host cell can be determined for the particular host cell envisioned without undue experimentation. “Period of time and under such conditions that cell growth is maximized” and the like, refer to those culture conditions that, for a particular cell line, are determined to be optimal for cell growth and division. For certain cell cultures (e.g. of mammalian cells) during the growth phase, cells are cultured in nutrient medium containing the necessary additives generally at about 30°-40° C. in a humidified, controlled atmosphere, such that optimal growth is achieved for the particular cell line. Cells may be maintained in the growth phase for a period of about between one and four days, usually between two to three days.

The term “transition phase” of the cell culture refers to the period of time during which culture conditions for the production phase are engaged. During the transition phase environmental factors such as temperature of the cell culture, medium osmolality and the like are shifted from growth conditions to production conditions.

The term “production phase” of the cell culture refers to the period of time during which cell growth is/has plateaued. The logarithmic cell growth typically decreases before or during this phase and protein production takes over. During the production phase, logarithmic cell growth has ended, and production of a product (e.g. a polypeptide) is primary. During this period of time, the medium is generally supplemented to support continued protein production and to achieve the desired product, which may be a glycoprotein. Fed-batch and/or perfusion cell culture processes supplement the cell culture medium or provide fresh medium during this phase to achieve and/or maintain desired cell density, viability and/or recombinant protein product titer. A production phase can be conducted at large scale.

The terms “expression” or “expresses” are used herein to refer to transcription and translation occurring within a host cell. The level of expression of a product gene in a host cell can be determined on the basis of either the amount of corresponding mRNA that is present in the cell or the amount of the protein encoded by the product gene that is produced by the cell. For example, mRNA transcribed from a product gene may be quantified by northern hybridization. Sambrook et al., Molecular Cloning: A Laboratory Manual, pp. 7.3-7.57 (Cold Spring Harbor Laboratory Press, 1989). Protein encoded by a product gene can be quantified either by assaying for the biological activity of the protein or by employing assays that are independent of such activity, such as western blotting or radioimmunoassay using antibodies that are capable of reacting with the protein. Sambrook et al., Molecular Cloning: A Laboratory Manual, pp. 18.1-18.88 (Cold Spring Harbor Laboratory Press, 1989).

The term “cell density” refers to the number of cells in a given volume of medium. In certain embodiments, a high cell density is desirable in that it can lead to higher protein productivity. Cell density can be monitored by any technique known in the art, including, but not limited to, extracting samples from a culture and analyzing the cells under a microscope, using a commercially available cell counting device or by using a commercially available suitable probe introduced into the bioreactor itself (or into a loop through which the medium and suspended cells are passed and then returned to the bioreactor).

### 5.2. Methods for Modulating BAX and BAK Expression

Provided herein are methods of reducing apoptotic activity in a eukaryotic cell by employing a genetic engineering system to modulate (i.e. knock down or knock out) (a) the expression of a Bax polypeptide isoform; and (b) the expression of a Bak polypeptide isoform. This also provides a stable integrated loss-of-function mutation or stable attenuation-of-function mutation in each of the Bax and Bak genes by introducing the mutation into any eukaryotic host cell that allows for the stable integration of the loss-of-function mutation or attenuation-of-function mutation into the eukaryotic host cell. A eukaryotic host cell that allows for the stable integration may be generated by a variety of methods including target integration (TI) (e.g. as described in WO 2019/126634), random integration (RI) or transposase mediated integration. Various genetic engineering systems known in the art can be used for said loss-of-function or attenuation-of-function engineering. Non-limiting examples of such engineering systems include the CRISPR/Cas system, the zinc-finger nuclease (ZFN) system, the transcription activator-like effector nuclease (TALEN) system. Any CRISPR/Cas systems known in the art, including traditional, enhanced or modified Cas systems, as well as other bacterial based genome excising tools such as Cpf-1 can be used with the methods disclosed herein.

In certain embodiments, the cells of the present disclosure exhibit reduced or eliminated expression of BAX. In certain embodiments, BAX, as used herein, refers to a eukaryotic BAX cellular protein, e.g., the CHO BAX cellular protein (Entrez Gene ID: 100689032; GenBank ID: EF104643.1), and functional variants thereof. In certain embodiments, functional variants of BAX, as used herein encompass BAX sequence variants having 50%, 55%, 60%, 65%, 70%, 75%, 80%, 85%, 90%, 91%, 92%, 93%, 94%, 95%, 96%, 97%, 98%, 99% identity to the wild type BAX sequence of the modified cell used for the production of a recombinant product of interest.

In certain embodiments, the cells of the present disclosure exhibit reduced or eliminated expression of BAK. In certain embodiments, BAK, as used herein, refers to a eukaryotic BAK cellular protein, e.g., the CHO BAK cellular protein (GenBank ID: EF104644.1), and functional variants thereof. In certain embodiments, functional variants of BAK, as used herein encompass BAK sequence variants having 50%, 55%, 60%, 65%, 70%, 75%, 80%, 85%, 90%, 91%, 92%, 93%, 94%, 95%, 96%, 97%, 98%, 99% identity to the wild type BAK sequence of the modified cell used for the production of a recombinant product of interest.

The entire gene or a portion of each of the Bax gene and/or Bak gene may be deleted to modulate, e.g., knock down or knock out, expression of a Bax polypeptide and/or Bak polypeptide. At least about 2%, at least about 5%, at least about 10%, at least about 15%, at least about 20%, at least about 25%, at least about 30%, at least about 35%, at least about 40%, at least about 45%, at least about 50%, at least about 55%, at least about 60%, at least about 65%, at least about 70%, at least about 75%, at least about 80%, at least about 85% or at least about 90% of the Bax gene may be deleted. At least about 2%, at least about 5%, at least about 10%, at least about 15%, at least about 20%, at least about 25%, at least about 30%, at least about 35%, at least about 40%, at least about 45%, at least about 50%, at least about 55%, at least about 60%, at least about 65%, at least about 70%, at least about 75%, at least about 80%, at least about 85% or at least about 90% of the Bak gene may be deleted. At least about 2%, at least about 5%, at least about 10%, at least about 15%, at least about 20%, at least about 25%, at least about 30%, at least about 35%, at least about 40%, at least about 45%, at least about 50%, at least about 55%, at least about 60%, at least about 65%, at least about 70%, at least about 75%, at least about 80%, at least about 85% or at least about 90% of the each of the Bax gene and Bak gene may be deleted.

No more than about 2%, no more than about 5%, no more than about 10%, no more than about 15%, no more than about 20%, no more than about 25%, no more than about 30%, no more than about 35%, no more than about 40%, no more than about 45%, no more than about 50%, no more than about 55%, no more than about 60%, no more than about 65%, no more than about 70%, no more than about 75%, no more than about 80%, no more than about 85% or no more than about 90% of the Bax gene may be deleted. No more than about 2%, no more than about 5%, no more than about 10%, no more than about 15%, no more than about 20%, no more than about 25%, no more than about 30%, no more than about 35%, no more than about 40%, no more than about 45%, no more than about 50%, no more than about 55%, no more than about 60%, no more than about 65%, no more than about 70%, no more than about 75%, no more than about 80%, no more than about 85% or no more than about 90% of the Bak gene may be deleted. No more than about 2%, no more than about 5%, no more than about 10%, no more than about 15%, no more than about 20%, no more than about 25%, no more than about 30%, no more than about 35%, no more than about 40%, no more than about 45%, no more than about 50%, no more than about 55%, no more than about 60%, no more than about 65%, no more than about 70%, no more than about 75%, no more than about 80%, no more than about 85% or no more than about 90% of each of the Bax gene and Bakgene may be deleted.

In certain examples, between about 2% and about 90%, between about 10% and about 90%, between about 20% and about 90%, between about 25% and about 90%, between about 30% and about 90%, between about 40% and about 90%, between about 50% and about 90%, between about 60% and about 90%, between about 70% and about 90%, between about 80% and about 90%, between about 85% and about 90%, between about 2% and about 80%, between about 10% and about 80%, between about 20% and about 80%, between about 30% and about 80%, between about 40% and about 80%, between about 50% and about 80%, between about 60% and about 80%, between about 70% and about 80%, between about 75% and about 80%, between about 2% and about 70%, between about 10% and about 70%, between about 20% and about 70%, between about 30% and about 70%, between about 40% and about 70%, between about 50% and about 70%, between about 60% and about 70%, between about 65% and about 70%, between about 2% and about 60%, between about 10% and about 60%, between about 20% and about 60%, between about 30% and about 60%, between about 40% and about 60%, between about 50% and about 60%, between about 55% and about 60%, between about 2% and about 50%, between about 10% and about 50%, between about 20% and about 50%, between about 30% and about 50%, between about 40% and about 50%, between about 45% and about 50%, between about 2% and about 40%, between about 10% and about 40%, between about 20% and about 40%, between about 30% and about 40%, between about 35% and about 40%, between about 2% and about 30%, between about 10% and about 30%, between about 20% and about 30%, between about 25% and about 30%, between about 2% and about 20%, between about 5% and about 20%, between about 10% and about 20%, between about 15% and about 20%, between about 2% and about 10%, between about 5% and about 10%, or between about 2% and about 5% of the Bax gene may be deleted. In certain examples, between about 2% and about 90%, between about 10% and about 90%, between about 20% and about 90%, between about 25% and about 90%, between about 30% and about 90%, between about 40% and about 90%, between about 50% and about 90%, between about 60% and about 90%, between about 70% and about 90%, between about 80% and about 90%, between about 85% and about 90%, between about 2% and about 80%, between about 10% and about 80%, between about 20% and about 80%, between about 30% and about 80%, between about 40% and about 80%, between about 50% and about 80%, between about 60% and about 80%, between about 70% and about 80%, between about 75% and about 80%, between about 2% and about 70%, between about 10% and about 70%, between about 20% and about 70%, between about 30% and about 70%, between about 40% and about 70%, between about 50% and about 70%, between about 60% and about 70%, between about 65% and about 70%, between about 2% and about 60%, between about 10% and about 60%, between about 20% and about 60%, between about 30% and about 60%, between about 40% and about 60%, between about 50% and about 60%, between about 55% and about 60%, between about 2% and about 50%, between about 10% and about 50%, between about 20% and about 50%, between about 30% and about 50%, between about 40% and about 50%, between about 45% and about 50%, between about 2% and about 40%, between about 10% and about 40%, between about 20% and about 40%, between about 30% and about 40%, between about 35% and about 40%, between about 2% and about 30%, between about 10% and about 30%, between about 20% and about 30%, between about 25% and about 30%, between about 2% and about 20%, between about 5% and about 20%, between about 10% and about 20%, between about 15% and about 20%, between about 2% and about 10%, between about 5% and about 10%, or between about 2% and about 5% of the Bak gene may be deleted. In certain examples, between about 2% and about 90%, between about 10% and about 90%, between about 20% and about 90%, between about 25% and about 90%, between about 30% and about 90%, between about 40% and about 90%, between about 50% and about 90%, between about 60% and about 90%, between about 70% and about 90%, between about 80% and about 90%, between about 85% and about 90%, between about 2% and about 80%, between about 10% and about 80%, between about 20% and about 80%, between about 30% and about 80%, between about 40% and about 80%, between about 50% and about 80%, between about 60% and about 80%, between about 70% and about 80%, between about 75% and about 80%, between about 2% and about 70%, between about 10% and about 70%, between about 20% and about 70%, between about 30% and about 70%, between about 40% and about 70%, between about 50% and about 70%, between about 60% and about 70%, between about 65% and about 70%, between about 2% and about 60%, between about 10% and about 60%, between about 20% and about 60%, between about 30% and about 60%, between about 40% and about 60%, between about 50% and about 60%, between about 55% and about 60%, between about 2% and about 50%, between about 10% and about 50%, between about 20% and about 50%, between about 30% and about 50%, between about 40% and about 50%, between about 45% and about 50%, between about 2% and about 40%, between about 10% and about 40%, between about 20% and about 40%, between about 30% and about 40%, between about 35% and about 40%, between about 2% and about 30%, between about 10% and about 30%, between about 20% and about 30%, between about 25% and about 30%, between about 2% and about 20%, between about 5% and about 20%, between about 10% and about 20%, between about 15% and about 20%, between about 2% and about 10%, between about 5% and about 10%, or between about 2% and about 5% of each of the Bax gene and Bak gene may be deleted.

A CRISPR/Cas9 system may be employed to modulate the expression of a Bax polypeptide and/or Bak polypeptide. A clustered regularly-interspaced short palindromic repeats (CRISPR) system is a genome editing tool discovered in prokaryotic cells. When utilized for genome editing, the system includes Cas9 (a protein able to modify DNA utilizing crRNA as its guide), CRISPR RNA (crRNA, contains the RNA used by Cas9 to guide it to the correct section of host DNA along with a region that binds to tracrRNA (generally in a hairpin loop form) forming an active complex with Cas9), and trans-activating crRNA (tracrRNA, binds to crRNA and forms an active complex with Cas9). The terms “guide RNA” and “gRNA” refer to any nucleic acid that promotes the specific association (or “targeting”) of an RNA-guided nuclease such as a Cas9 to a target sequence such as a genomic or episomal sequence in a cell. gRNAs can be unimolecular (comprising a single RNA molecule, and referred to alternatively as chimeric) or modular (comprising more than one, and typically two, separate RNA molecules, such as a crRNA and a tracrRNA, which are usually associated with one another, for instance by duplexing). CRISPR/Cas9 strategies can employ a vector to transfect the mammalian cell. The guide RNA (gRNA) can be designed for each application as this is the sequence that Cas9 uses to identify and directly bind to the target DNA in a cell. Multiple crRNAs and the tracrRNA can be packaged together to form a single-guide RNA (sgRNA). The sgRNA can be joined together with the Cas9 gene and made into a vector in order to be transfected into cells.

A CRISPR/Cas9 system for use in modulating expression of one or more Bax polypeptides and/or Bak polypeptides may comprise a Cas9 molecule and one or more gRNAs comprising a targeting domain that is complementary to a target sequence of the Bax gene and/or Bak gene. The target gene may be a region of the Bax gene and/or the Bak gene. The target sequence can thus be any exon or intron region within the Bax gene, e.g., the targeting of which eliminates or reduces the expression of a Bax polypeptide. The target sequence can thus be any exon or intron region within the Bak gene, e.g., the targeting of which eliminates or reduces the expression of a Bak polypeptide.

The gRNAs may be administered to the cell in a single vector and the Cas9 molecule may be administered to the cell in a second vector. The gRNAs and the Cas9 molecule may be administered to the cell in a single vector. Alternatively, each of the gRNAs and Cas9 molecule may be administered by separate vectors. In examples, the CRISPR/Cas9 system can be delivered to the cell as a ribonucleoprotein complex (RNP) that comprises a Cas9 protein complexed with one or more gRNAs, e.g., delivered by electroporation (see, e.g., DeWitt et al., Methods 121-122:9-15 (2017) for additional methods of delivering RNPs to a cell). Administering the CRISPR/Cas9 system to the cell typically results in the knock out or knock down of the expression of both the Bax and Bak polypeptides.

The genetic engineering system used for modulating the expression of a Bax polypeptide and/or a Bak polypeptide may be a ZFN system. The ZFN can act as restriction enzyme, which is generated by combining a zinc finger DNA-binding domain with a DNA-cleavage domain. A zinc finger domain can be engineered to target specific DNA sequences which allows the zinc-finger nuclease to target desired sequences within genomes. The DNA-binding domains of individual ZFNs typically contain a plurality of individual zinc finger repeats and can each recognize a plurality of base pairs. The most common method to generate a new zinc-finger domain is to combine smaller zinc-finger “modules” of known specificity. The most common cleavage domain in ZFNs is the non-specific cleavage domain from the type IIs restriction endonuclease FokI. ZFN modulates the expression of proteins by producing double-strand breaks (DSBs) in the target DNA sequence, which will, in the absence of a homologous template, be repaired by non-homologous end-joining (NHEJ). Such repair can result in deletion or insertion of base-pairs, producing frame-shift and preventing the production of the harmful protein (Durai et al., Nucleic Acids Res.; 33 (18): 5978-90 (2005)). Multiple pairs of ZFNs can also be used to completely remove entire large segments of genomic sequence (Lee et al., Genome Res.; 20 (1): 81-9 (2010)). The target gene may be part of the Bax gene. The target gene may be part of the Bak gene.

The genetic engineering system used for modulating the expression of a Bax polypeptide and/or a Bak polypeptide may be a TALEN system. TALENs are restriction enzymes that can be engineered to cut specific sequences of DNA. TALEN systems operate on a similar principle as ZFNs. TALENs are generated by combining a transcription activator-like effectors DNA-binding domain with a DNA cleavage domain. Transcription activator-like effectors (TALEs) are composed of 33-34 amino acid repeating motifs with two variable positions that have a strong recognition for specific nucleotides. By assembling arrays of these TALEs, the TALE DNA-binding domain can be engineered to bind desired DNA sequence, and thereby guide the nuclease to cut at specific locations in genome (Boch et al., Nature Biotechnology; 29(2):135-6 (2011 The target gene may be part of the Bax gene. The target gene may be part of the Bak gene.

The genetic engineering system disclosed herein can be delivered into the mammalian cell using a viral vector, e.g., retroviral vectors such as gamma-retroviral vectors, and lentiviral vectors. Combinations of retroviral vector and an appropriate packaging line are suitable, where the capsid proteins will be functional for infecting human cells. Various amphotropic virus-producing cell lines are known, including, but not limited to, PA12 (Miller, et al. (1985) Mol. Cell. Biol. 5:431-437); PA317 (Miller, et al. (1986) Mol. Cell. Biol. 6:2895-2902); and CRIP (Danos, et al. (1988) Proc. Natl. Acad. Sci. USA 85:6460-6464). Non-amphotropic particles are suitable too, e.g., particles pseudotyped with VSVG, RD114 or GALV envelope and any other known in the art. Possible methods of transduction also include direct co-culture of the cells with producer cells, e.g., by the method of Bregni, et al. (1992) Blood 80:1418-1422, or culturing with viral supernatant alone or concentrated vector stocks with or without appropriate growth factors and polycations, e.g., by the method of Xu, et al. (1994) Exp. Hemat. 22:223-230; and Hughes, et al. (1992) J. Clin. Invest. 89:1817.

Other transducing viral vectors can be used to modify the mammalian cell disclosed herein. In certain embodiments, the chosen vector exhibits high efficiency of infection and stable integration and expression (see, e.g., Cayouette et al., Human Gene Therapy 8:423-430, 1997; Kido et al., Current Eye Research 15:833-844, 1996; Bloomer et al., Journal of Virology 71:6641-6649, 1997; Naldini et al., Science 272:263-267, 1996; and Miyoshi et al., Proc. Natl. Acad. Sci. U.S.A. 94:10319, 1997). Other viral vectors that can be used include, for example, adenoviral, lentiviral, and adena-associated viral vectors, vaccinia virus, a bovine papilloma virus, or a herpes virus, such as Epstein-Barr Virus (also see, for example, the vectors of Miller, Human Gene Therapy 15-14, 1990; Friedman, Science 244:1275-1281, 1989; Eglitis et al., BioTechniques 6:608-614, 1988; Tolstoshev et al., Current Opinion in Biotechnology 1:55-61, 1990; Sharp, The Lancet 337:1277-1278, 1991; Cornetta et al., Nucleic Acid Research and Molecular Biology 36:311-322, 1987; Anderson, Science 226:401-409, 1984; Moen, Blood Cells 17:407-416, 1991; Miller et al., Biotechnology 7:980-990, 1989; LeGal La Salle et al., Science 259:988-990, 1993; and Johnson, Chest 107:77S-83S, 1995). Retroviral vectors are particularly well developed and have been used in clinical settings (Rosenberg et al., N. Engl. J. Med 323:370, 1990; Anderson et al., U.S. Pat. No. 5,399,346).

### 5.3 Cells Lines

The disclosure relates to an isolated cell line, wherein the cell line comprises a stable integrated loss-of-function or attenuation-of-function mutation in each of the Bax and Bak genes. In an aspect, the isolated cell line is a eukaryotic cell line.

In certain embodiments, the cell line comprises a stable integrated loss-of-function mutation in each of the Bax and Bak genes.

In certain embodiments, the cell line comprises a deletion in each of the Bax and Bak genes.

In certain embodiments, the cell line is an animal cell line or a fungal cell line. The cell line may be an animal cell line, e.g. a mammalian cell line. Exemplary mammalian cell lines include hybridoma cell lines, CHO cell lines, COS cell lines, VERO cell lines, HeLa cell lines, HEK 293 cell lines, PER-C6 cell lines, K562 cell lines, MOLT-4 cell lines, Ml cell lines, NS-1 cell lines, COS-7 cell lines, MDBK cell lines, MDCK cell lines, MRC-5 cell lines, WI-38 cell lines, WEHI cell lines, SP2/0 cell lines, BHK cell lines (including BHK-21 cell lines), or their derivatives. The cell line may be a CHO cell line, e.g. a CHO K1 cell line, a CHO K1SV cell line, a DG44 cell line, a DUKXB-11 cell line, a CHOK1S cell line, or a CHO K1M cell line, or their derivatives. The cell line may be a fungal cell line, e.g. a yeast cell line.

In certain embodiments, the cell line further comprises a viral genome and one or more polynucleotides encoding a viral capsid.

In certain embodiments, the cell line further comprises a polynucleotide encoding a product of interest.

The polynucleotide that encodes the product of interest may be integrated in the cellular genome of the cell line at a targeted location. The polynucleotide that encodes the product of interest may be randomly integrated in the cellular genome of the cell line. The polynucleotide that encodes the product of interest may be an extrachromosomal polynucleotide. The polynucleotide that encodes the product of interest may be integrated into a chromosome of the cell line.

In certain embodiments, the polynucleotide that encodes the product of interest may be integrated in the cellular genome of the cell line at a targeted location. Such targeted integration allows for exogenous nucleotide sequences to be integrated into one or more pre-determined sites of a host cell genome. In certain embodiments, the targeted integration is mediated by a recombinase that recognizes one or more recombinant recognition sequences (RRSs). The RRS or RRSs may be selected from the group consisting of a LoxP sequence, a LoxP L3 sequence, a LoxP 2L sequence, a LoxFas sequence, a Lox511 sequence, a Lox2272 sequence, a Lox2372 sequence, a Lox5171 sequence, a Loxm2 sequence, a Lox71 sequence, a Lox66 sequence, a FRT sequence, a Bxb1 attP sequence, a Bxb1 attB sequence, a φC31 attP sequence, and a φC31 attB sequence. The targeted integration may be mediated by homologous recombination. The targeted integration may be mediated by an exogenous site-specific nuclease followed by homology-directed repair (HDR) and/or non-homologous end joining (NHEJ). Targeted integration in accordance with the present disclosure may be as further described in WO 2019/126634 (see, e.g., WO 2019/126634 sections 5.1, 5.2, 5.3 and 5.4, on pages 42-55; with methods of preparing cells using targeted integration further described in sections 6.1 and 6.2 on pages 55-67).

In certain embodiments employing targeted integration, the exogenous nucleotide sequence is integrated at a site within a specific locus of the genome of a host cell, (a “TI host cell”). In certain embodiments, the locus into which the exogenous nucleotide sequence is integrated is at least about 50%, at least about 60%, at least about 70%, at least about 80%, at least about 90%, at least about 95%, at least about 99%, or at least about 99.9% homologous to a sequence selected from Contigs NW_006874047.1, NW_006884592.1, NW_006881296.1, NW_003616412.1, NW_003615063.1, NW_006882936.1, and NW_003615411.1.

In certain embodiments, the nucleotide sequence immediately 5′ of the integrated exogenous sequence is selected from the group consisting of nucleotides 41190-45269 of NW_006874047.1, nucleotides 63590-207911 of NW_006884592.1, nucleotides 253831-491909 of NW_006881296.1, nucleotides 69303-79768 of NW_003616412.1, nucleotides 293481-315265 of NW_003615063.1, nucleotides 2650443-2662054 of NW_006882936.1, or nucleotides 82214-97705 of NW_003615411.1 and sequences at least 50% homologous thereto. In certain embodiments, the nucleotide sequence immediately 5′ of the integrated exogenous sequence are at least about 50%, at least about 60%, at least about 70%, at least about 80%, at least about 90%, at least about 95%, at least about 99%, or at least about 99.9% homologous to nucleotides 41190-45269 of NW_006874047.1, nucleotides 63590-207911 of NW_006884592.1, nucleotides 253831-491909 of NW_006881296.1, nucleotides 69303-79768 of NW_003616412.1, nucleotides 293481-315265 of NW_003615063.1, nucleotides 2650443-2662054 of NW_006882936.1, or nucleotides 82214-97705 of NW_003615411.1.

In certain embodiments, the nucleotide sequence immediately 3′ of the integrated exogenous sequence is selected from the group consisting of nucleotides 45270-45490 of NW_006874047.1, nucleotides 207912-792374 of NW_006884592.1, nucleotides 491910-667813 of NW_006881296.1, nucleotides 79769-100059 of NW_003616412.1, nucleotides 315266-362442 of NW_003615063.1, nucleotides 2662055-2701768 of NW_006882936.1, or nucleotides 97706-105117 of NW_003615411.1 and sequences at least 50% homologous thereto. In certain embodiments, the nucleotide sequence immediately 3′ of the integrated exogenous sequence is at least about 50%, at least about 60%, at least about 70%, at least about 80%, at least about 90%, at least about 95%, at least about 99%, or at least about 99.9% homologous to nucleotides 45270-45490 of NW_006874047.1, nucleotides 207912-792374 of NW_006884592.1, nucleotides 491910-667813 of NW_006881296.1, nucleotides 79769-100059 of NW_003616412.1, nucleotides 315266-362442 of NW_003615063.1, nucleotides 2662055-2701768 of NW_006882936.1, or nucleotides 97706-105117 of NW_003615411.1.

In certain embodiments, the integrated exogenous sequence is flanked 5′ by a nucleotide sequence selected from the group consisting of nucleotides 41190-45269 of NW_006874047.1, nucleotides 63590-207911 of NW_006884592.1, nucleotides 253831-491909 of NW_006881296.1, nucleotides 69303-79768 of NW_003616412.1, nucleotides 293481-315265 of NW_003615063.1, nucleotides 2650443-2662054 of NW_006882936.1, and nucleotides 82214-97705 of NW_003615411.1. and sequences at least 50% homologous thereto. In certain embodiments, the integrated exogenous sequence is flanked 3′ by a nucleotide sequence selected from the group consisting of nucleotides 45270-45490 of NW_006874047.1, nucleotides 207912-792374 of NW_006884592.1, nucleotides 491910-667813 of NW_006881296.1, nucleotides 79769-100059 of NW_003616412.1, nucleotides 315266-362442 of NW_003615063.1, nucleotides 2662055-2701768 of NW_006882936.1, and nucleotides 97706-105117 of NW_003615411.1 and sequences at least 50% homologous thereto. In certain embodiments, the nucleotide sequence flanking 5′ of the integrated exogenous nucleotide sequence is at least about 50%, at least about 60%, at least about 70%, at least about 80%, at least about 90%, at least about 95%, at least about 99%, or at least about 99.9% homologous to nucleotides 41190-45269 of NW_006874047.1, nucleotides 63590-207911 of NW_006884592.1, nucleotides 253831-491909 of NW_006881296.1, nucleotides 69303-79768 of NW_003616412.1, nucleotides 293481-315265 of NW_003615063.1, nucleotides 2650443-2662054 of NW_006882936.1, and nucleotides 82214-97705 of NW_003615411.1. In certain embodiments, the nucleotide sequence flanking 3′ of the integrated exogenous nucleotide sequence is at least about 50%, at least about 60%, at least about 70%, at least about 80%, at least about 90%, at least about 95%, at least about 99%, or at least about 99.9% homologous to nucleotides 45270-45490 of NW_006874047.1, nucleotides 207912-792374 of NW_006884592.1, nucleotides 491910-667813 of NW_006881296.1, nucleotides 79769-100059 of NW_003616412.1, nucleotides 315266-362442 of NW_003615063.1, nucleotides 2662055-2701768 of NW_006882936.1, and nucleotides 97706-105117 of NW_003615411.1.

In certain embodiments, the integrated exogenous nucleotide sequence is operably linked to a nucleotide sequence selected from the group consisting of Contigs NW_006874047.1, NW_006884592.1, NW_006881296.1, NW_003616412.1, NW_003615063.1, NW_006882936.1, and NW_003615411.1 and sequences at least 50% homologous thereto. In certain embodiments, the nucleotide sequence operably linked to the exogenous nucleotide sequence is at least about 50%, at least about 60%, at least about 70%, at least about 80%, at least about 90%, at least about 95%, at least about 99%, or at least about 99.9% homologous to a sequence selected from Contigs NW_006874047.1, NW_006884592.1, NW_006881296.1, NW_003616412.1, NW_003615063.1, NW_006882936.1, and NW_003615411.1.

In certain embodiments, the nucleic acid encoding a product of interest can be integrated into a host cell genome using transposase-based integration. Transposase-based integration techniques are disclosed, for example, in Trubitsyna et al., Nucleic Acids Res. 45(10):e89 (2017), Li et al., PNAS 110(25):E2279-E2287 (2013) and WO 2004/009792, which are incorporated by reference herein in their entireties.

The product of interest may be or comprise a recombinant polypeptide. The product of interest (such as a recombinant polypeptide) may be or comprise an antibody, an antigen, an enzyme, or a vaccine. The antibody may be a multispecific antibody or antigen-binding fragment thereof. The antibody may be a multispecific antibody or antigen-binding fragment thereof. The antibody may consist of a single heavy chain sequence and a single light chain sequence or antigen-binding fragments thereof. The antibody may comprise a chimeric antibody, a human antibody or a humanized antibody. The product of interest may be a complex molecule, for e.g., a part antibody and part protein, or a non-antibody complex protein and such derivatives. The antibody may comprise a monoclonal antibody.

In certain embodiments, the cell line has a higher specific productivity than a corresponding eukaryotic cell line that comprises the polynucleotide and functional copies of each of the wild type Bax and Bak genes. For example, the cell line may have a specific productivity (Qp) that is at least about 5%, at least about 10%, at least about 15%, at least about 20%, at least about 25%, at least about 30%, at least about 35%, at least about 40%, at least about 45%, at least about 50%, at least about 55%, or at least about 60% higher than the specific productivity of the corresponding eukaryotic cell line that comprises the polynucleotide and functional copies of each of the wild type Bax and Bak genes. For example, the cell line may have a titre of the product of interest that is at least about 5%, at least about 10%, at least about 15%, at least about 20%, at least about 25%, at least about 30%, at least about 35%, at least about 40%, at least about 45%, at least about 50%, at least about 55%, or at least about 60% higher than the titre of the corresponding eukaryotic cell line that comprises the polynucleotide and functional copies of each of the wild type Bax and Bak genes.

In certain embodiments, the cell line is more resistant to apoptosis than a corresponding isolated eukaryotic cell line that comprises functional copies of each of the Bax and Bak genes.

### 5.4. Cell Cultures

A cell culture comprises a cell culture medium and at least one (typically a plurality of) cells. For example, a cell culture medium may comprise a cell culture medium and a plurality of eukaryotic cells, wherein each cell of the plurality comprises a stable integrated loss-of-function or attenuation-of-function mutation in each of the Bax and Bak genes.

Cell culture media contain many components. Cell culture media provide the nutrients necessary to maintain and grow cells in a controlled, artificial and in vitro environment. Characteristics and compositions of the cell culture media vary depending on the particular cellular requirements. Important parameters include osmolarity, pH, and nutrient formulations.

Culture media contain a mixture of amino acids, glucose, salts, vitamins, and other nutrients, and are available either as a powder or as a liquid form from commercial suppliers. The requirements for these components vary among cell lines. Regulation of pH is critical for optimum culture conditions and is generally achieved using a suitable buffering system. While chemically defined media (CDM) are preferred for therapeutic and related applications, as CDM provide reproducible contamination-free media when prepared and used under aseptic conditions, for some cell types it may be necessary to use media comprising serum, proteins or other biological extracts (such as yeast extracts or enzymatic digests of plant or animal matter).

Some extremely simple defined media, which consist essentially of vitamins, amino acids, organic and inorganic salts and buffers have been used for cell culture. Such media (often called “basal media”), however, are usually seriously deficient in the nutritional content required by most animal cells. These media therefore often need to be supplemented, for example with feeds or other additives, to form complete media. In addition, batch culture systems often include periodic supplementation of the media with concentrated feeds or additives, to maintain the viability of cultured cells and/or production of biological products, such as polypeptides (e.g. antibodies, or biologically functional fragments of antibodies), proteins, peptides, hormones, viruses or virus like particles, nucleic acids or fragments thereof.

Ingredients that may be present in basal media include amino acids (nitrogen source), vitamins, inorganic salts, sugars (carbon source), buffering salts and lipids. Basal media for use with some mammalian cell culture systems may contain ethanolamine, D-glucose, N-[2-hydroxyethyl]-piperazine-N′-[2-ethanesulfonic acid](HEPES), linoleic acid, lipoic acid, phenol red, PLURONIC F68, putrescine, sodium pyruvate.

Amino acid ingredients which may be included in the media include L-alanine, L-arginine, L-asparagine, L-aspartic acid, L-cysteine, L-glutamic acid, L glutamine, glycine, L-histidine, L-isoleucine, L-leucine, L-lysine, L-methionine, L phenylalanine, L-proline, L-serine, L-threonine, L-tryptophan, L-tyrosine, L-valine, and derivatives thereof. These amino acids may be obtained commercially, for example from Sigma (Saint Louis, Missouri).

Vitamin ingredients which may be included in the media include biotin, choline chloride, D-Ca2+-pantothenate, folic acid, i-inositol, niacinamide, pyridoxine, riboflavin, thiamine and vitamin B12. These vitamins may be obtained commercially, for example from Sigma (Saint Louis, Missouri).

Inorganic salt ingredients which may be used in the media include one or more calcium salts (e.g., CaCl2)), Fe(NO3)3, KCl, one or more magnesium salts (e.g., MgCl2 and/or MgSO4), one or more manganese salts (e.g., MnCl2), NaCl, NaHCO3, N2HPO4, and ions of the trace elements selenium, vanadium, zinc and copper. These trace elements may be provided in a variety of forms, preferably in the form of salts such as Na2SeO3, NH4VO3, ZnSO4 and CuSO4. These inorganic salts and trace elements may be obtained commercially, for example from Sigma (Saint Louis, Missouri).

Exemplary media that are useful in the culture of mammalian cells include commercially available media such as Ham's F10 (Sigma), Minimal Essential Medium ((MEM), Sigma), RPMI-1640 (Sigma), and Dulbecco's Modified Eagle's Medium ((DMEM, Sigma) are suitable for culturing the host cells. In addition, any of the media described in Ham and Wallace (1979), Meth. in Enz. 58:44; Barnes and Sato (1980), Anal. Biochem. 102:255; U.S. Pat. Nos. 4,767,704; 4,657,866; 4,927,762; or 4,560,655; WO 90/03430; WO 87/00195; U.S. Pat. No. Re. 30,985; or U.S. Pat. No. 5,122,469, the disclosures of all of which are incorporated herein by reference, may be used as culture media for the host cells. Any of these media may be supplemented as necessary with hormones and/or other growth factors (such as insulin, transferrin, or epidermal growth factor), salts (such as sodium chloride, calcium, magnesium, and phosphate), buffers (such as HEPES), nucleosides (such as adenosine and thymidine), antibiotics (such as Gentamycin™ drug), trace elements (defined as inorganic compounds usually present at final concentrations in the micromolar range), and glucose or an equivalent energy source. Any other necessary supplements may also be included at appropriate concentrations that would be known to those skilled in the art. The culture conditions, such as temperature, pH, and the like, are those previously used with the host cell selected for expression, and will be apparent to the ordinarily skilled artisan. Exemplary culture conditions are provided in M. Takagi and K. Ueda, “Comparison of the optimal culture conditions for cell growth and tissue plasminogen activator production by human embryo lung cells on microcarriers”, Biotechnology, (1994), 41, 565-570; H. J. Morton, “A survey of commercially available tissue culture media”, In Vitro (1970), 6(2), 89-108; J. Van der Valk, et al., (2010), “Optimization of chemically defined cell culture media-replacing fetal bovine serum in mammalian in vitro methods,” Toxicology in vitro, 24(4), 1053-1063; R. J. Graham et al., “Consequences of trace metal variability and supplementation on Chinese hamster ovary (CHO) cell culture performance: A review of key mechanisms and considerations”, BIotechnol. Bioeng. (2019), 116(12), 3446-3456; S. Janoschek et al., A protocol to transfer a fed-batch platform process into semi-perfusion mode: The benefit of automated small-scale bioreactors compared to shake flasks as scale-down model”, Biotechnol. Prog., (2019), 35(2), e2757; and M. Kuiper et al., “Repurposing fed-batch media and feeds for highly productive CHO perfusion processes”, Biotechnology Progress, 15 Apr. 2019, https://doi.org/10.1002/btpr.2821; all of which are incorporated by reference herein in their entirety.

Exemplary media that are useful for the production of CHO cells can contain a basal medium component such as a DMEM/HAM F-12 based formulation (for composition of DMEM and HAM F12 media, see culture media formulations in American Type Culture Collection Catalogue of Cell Lines and Hybridomas, Sixth Edition, 1988, pages 346-349) (the formulation of medium as described in U.S. Pat. No. 5,122,469 are particularly appropriate) with modified concentrations of some components such as amino acids, salts, sugar, and vitamins, and optionally containing glycine, hypoxanthine, and thymidine; recombinant human insulin, hydrolyzed peptone, such as Primatone HS or Primatone RL (Sheffield, England), or the equivalent; a cell protective agent, such as Pluronic F68 or the equivalent pluronic polyol; gentamycin; and trace elements.

The cell culture may comprise eukaryotic cells comprises a stable integrated loss-of-function or attenuation-of-function mutation in each of the Bax and Bak genes, which express a recombinant protein. The recombinant protein can be produced by growing cells which express the products of interest under a variety of cell culture conditions. For instance, cell culture procedures for the large or small-scale production of proteins are potentially useful within the context of the present disclosure. Procedures including, but not limited to, a fluidized bed bioreactor, hollow fiber bioreactor, roller bottle culture, shake flask culture, or stirred tank bioreactor system can be used, in the latter two systems, with or without microcarriers, and operated alternatively in a batch, fed-batch, or continuous mode

The cell culture of the present disclosure may be performed in a stirred tank bioreactor system and a fed batch culture procedure is employed. In the fed batch culture, the eukaryotic host cells (e.g. mammalian host cells) and culture medium are supplied to a culturing vessel initially and additional culture nutrients are fed, continuously or in discrete increments, to the culture during culturing, with or without periodic cell and/or product harvest before termination of culture. The fed batch culture can include, for example, a semi-continuous fed batch culture, wherein periodically whole culture (including cells and medium) is removed and replaced by fresh medium. Fed batch culture is distinguished from simple batch culture in which all components for cell culturing (including the cells and all culture nutrients) are supplied to the culturing vessel at the start of the culturing process. Fed batch culture can be further distinguished from perfusion culturing insofar as the supernatant is not removed from the culturing vessel during the process (in perfusion culturing, the cells are restrained in the culture by, e.g., filtration, encapsulation, anchoring to microcarriers etc. and the culture medium is continuously or intermittently introduced and removed from the culturing vessel).

The cells of the culture may be propagated according to any scheme or routine that can be suitable for the specific host cell and the specific production plan contemplated. Therefore, the present disclosure contemplates a single step or multiple step culture procedure. In a single step culture, the host cells are inoculated into a culture environment and the processes of the instant disclosure are employed during a single production phase of the cell culture. Alternatively, a multi-stage culture is envisioned. In the multi-stage culture cells can be cultivated in a number of steps or phases. For instance, cells can be grown in a first step or growth phase culture wherein cells, possibly removed from storage, are inoculated into a medium suitable for promoting growth and high viability. The cells can be maintained in the growth phase for a suitable period of time by the addition of fresh medium to the host cell culture.

Fed batch or continuous cell culture conditions are typically devised to enhance growth of the eukaryotic cells (e.g. mammalian cells) in the growth phase of the cell culture. In the growth phase cells are grown under conditions and for a period of time that is maximized for growth. Culture conditions, such as temperature, pH, dissolved oxygen (dO2) and the like, are those used with the particular host and will be apparent to the ordinarily skilled artisan. Generally, the pH is adjusted to a level between about 6.5 and 7.5 using either an acid (e.g., CO2) or a base (e.g., Na2CO3 or NaOH). A suitable temperature range for culturing mammalian cells such as CHO cells is between about 30° to 38° C. and a suitable dO2 is between 5-90% of air saturation.

At a particular stage the cells can be used to inoculate a production phase or step of the cell culture. Alternatively, as described above the production phase or step can be continuous with the inoculation or growth phase or step.

The culturing methods described in the present disclosure can further include harvesting the product from the cell culture, e.g., from the production phase of the cell culture. In certain embodiments, the product produced by the cell culture methods of the present disclosure can be harvested from the third bioreactor, e.g., production bioreactor. For example, but not by way of limitation, the disclosed methods can include harvesting the product at the completion of the production phase of the cell culture. Alternatively, or additionally, the product can be harvested prior to the completion of the production phase. In certain embodiments, the product can be harvested from the cell culture once a particular cell density has been achieved. For example, but not by way of limitation, the cell density can be from about 2.0×10⁷ cells/mL to about 5.0×10⁷ cells/mL prior to harvesting.

Harvesting or isolating the product from the cell culture can include one or more of centrifugation, filtration, acoustic wave separation, flocculation and cell removal technologies.

The product of interest can be secreted from the host cells or can be a membrane-bound, cytosolic or nuclear protein. Soluble forms of the polypeptide may be purified from the conditioned cell culture media and membrane-bound forms of the polypeptide can be purified by preparing a total membrane fraction from the expressing cells and extracting the membranes with a nonionic detergent such as TRITON® X-100 (EMD Biosciences, San Diego, Calif.). Cytosolic or nuclear proteins may be prepared by lysing the host cells (e.g., by mechanical force, sonication and/or detergent), removing the cell membrane fraction by centrifugation and retaining the supernatant.

In an embodiment, the invention provides a composition, comprising a eukaryotic cell line as disclosed herein wherein the cells of the cell line comprise a stable integrated loss-of-function or attenuation-of-function mutation in each of the Bax and Bak genes, and a cell culture medium as disclosed herein.

Another embodiment provides a cell culture comprising a cell culture medium and a plurality of eukaryotic cells, wherein each cell of the plurality comprises a stable integrated loss-of-function or attenuation-of-function mutation in each of the Bax and Bak genes. The cells may have further features as disclosed herein. The cell culture medium may be further defined as disclosed herein.

### 5.5 Methods of Production

In certain embodiments, the present disclosure provides methods of producing a recombinant polypeptide. In certain embodiments, the methods comprise culturing a eukaryotic cell line, under conditions suitable for production of the polypeptide. In certain embodiments, the cell line comprises: (a) a stable integrated loss-of-function or attenuation-of-function mutation in each of the Bax and Bak genes; and (b) a polynucleotide encoding the recombinant polypeptide.

The polynucleotide that encodes the polypeptide may be integrated in the cellular genome of the cells of the cell line at a targeted location. Such targeted integration allows for exogenous nucleotide sequences to be integrated into one or more pre-determined sites of a host cell genome. In certain embodiments, the targeted integration is mediated by a recombinase that recognizes one or more recombinant recognition sequences (RRSs). The RRS or RRSs may be selected from the group consisting of a LoxP sequence, a LoxP L3 sequence, a LoxP 2L sequence, a LoxFas sequence, a Lox511 sequence, a Lox2272 sequence, a Lox2372 sequence, a Lox5171 sequence, a Loxm2 sequence, a Lox71 sequence, a Lox66 sequence, a FRT sequence, a Bxb1 attP sequence, a Bxb1 attB sequence, a φC31 attP sequence, and a φC31 attB sequence. The targeted integration may be mediated by homologous recombination. The targeted integration may be mediated by an exogenous site-specific nuclease followed by homology-directed repair (HDR) and/or non-homologous end joining (NHEJ). Targeted integration in accordance with the present disclosure may be as further described in WO 2019/126634 (see, e.g., WO 2019/126634 sections 5.1, 5.2, 5.3 and 5.4, on pages 42-55; with methods of preparing cells using targeted integration further described in sections 6.1 and 6.2 on pages 55-67).

The polynucleotide that encodes the polypeptide may be randomly integrated in the cellular genome of the cells of the cell line. The polynucleotide that encodes the polypeptide may be an extrachromosomal polynucleotide. The polynucleotide that encodes the polypeptide may be integrated into a chromosome of the cells of the cell line.

The recombinant polypeptide may be or comprise an antibody, an antigen, an enzyme, or a vaccine. The recombinant polypeptide may be or comprise an antibody. The recombinant polypeptide may be or comprise an antigen. The recombinant polypeptide may be or comprise an enzyme. The recombinant polypeptide may be or comprise a vaccine. The antibody may be a multispecific antibody or antigen-binding fragment thereof. The antibody may be a multispecific antibody or antigen-binding fragment thereof. The antibody may consist of a single heavy chain sequence and a single light chain sequence or antigen-binding fragments thereof. The antibody may comprise a chimeric antibody, a human antibody or a humanized antibody. The antibody may comprise a monoclonal antibody.

The method can further comprise isolating the recombinant polypeptide. Such isolation typically comprises isolating the recombinant polypeptide from the cell line. Isolating the recombinant polypeptide can include one or more of centrifugation, filtration, acoustic wave separation, flocculation and cell removal technologies. The isolated recombinant polypeptide can be purified.

The cell line can be an animal cell line or a fungal cell line. The cell line can be an animal cell line, e.g. a mammalian cell line. Exemplary mammalian cell lines include hybridoma cell lines, CHO cell lines, COS cell lines, VERO cell lines, HeLa cell lines, HEK 293 cell lines, PER-C6 cell lines, K562 cell lines, MOLT-4 cell lines, Ml cell lines, NS-1 cell lines, COS-7 cell lines, MDBK cell lines, MDCK cell lines, MRC-5 cell lines, WI-38 cell lines, WEHI cell lines, SP2/0 cell lines, BHK cell lines (including BHK-21 cell lines), or their derivatives. The cell line may be a CHO cell line, e.g. a CHO K1 cell line, a CHO K1SV cell line, a DG44 cell line, a DUKXB-11 cell line, a CHOK1S cell line, or a CHO K1M cell line, or their derivatives. The cell line may be a fungal cell line, e.g. a yeast cell line.

The cell line can cultured in a cell culture medium. The cell culture medium and/or cell culture conditions may be as further described above, under the heading “Cell Cultures”. The cell line may be cultured under fed-batch culture conditions, or perfusion culture conditions. The cell line can be cultured under fed-batch culture conditions. The fed-batch culture conditions can be intensified fed-batch culture conditions. The cell line can be cultured under perfusion culture conditions. The perfusion culture conditions can be semi-continuous perfusion. The perfusion culture conditions can be continuous perfusion.

The cell line can comprise a stable integrated loss-of-function mutation in each of the Bax and Bak genes.

In certain embodiments, the present disclosure provides methods of producing a viral vector. In certain embodiments, the methods comprise culturing a eukaryotic cell line under conditions suitable for production of the viral vector. In certain embodiments, the cell line comprises (a) stable integrated a loss-of-function or attenuation-of function mutation in each of the Bax and Bak genes, (b) a viral genome, and (c) one or more polynucleotides encoding a viral capsid, under conditions suitable for production of the viral vector.

In certain embodiments, the methods can comprise isolating the viral vector. In certain embodiments, isolation typically comprises isolating the viral vector from the cell line. Isolating the viral vector may include one or more of centrifugation, filtration, acoustic wave separation, flocculation and cell removal technologies. The isolated viral vector may be purified.

The cell line may be an animal cell line or a fungal cell line. The cell line may be an animal cell line, e.g. a mammalian cell line. Exemplary mammalian cell lines include hybridoma cell lines, CHO cell lines, COS cell lines, VERO cell lines, HeLa cell lines, HEK 293 cell lines, PER-C6 cell lines, K562 cell lines, MOLT-4 cell lines, Ml cell lines, NS-1 cell lines, COS-7 cell lines, MDBK cell lines, MDCK cell lines, MRC-5 cell lines, WI-38 cell lines, WEHI cell lines, SP2/0 cell lines, BHK cell lines (including BHK-21 cell lines), or their derivatives. The cell line may be a CHO cell line, e.g. a CHO K1 cell line, a CHO K1SV cell line, a DG44 cell line, a DUKXB-11 cell line, a CHOK1S cell line, or a CHO K1M cell line, or their derivatives. The cell line may be a fungal cell line, e.g. a yeast cell line.

The cell line may cultured in a cell culture medium. The cell culture medium and/or cell culture conditions may be as further described above, under the heading “Cell Cultures”. The cell line may be cultured under fed-batch culture conditions, or perfusion culture conditions. The cell line may be cultured under fed-batch culture conditions. The fed-batch culture conditions may be intensified fed-batch culture conditions. The cell line may be cultured under perfusion culture conditions. The perfusion culture conditions may be semi-continuous perfusion. The perfusion culture conditions may be continuous perfusion.

In certain embodiments, the cell line can comprise a stable integrated loss-of-function mutation in each of the Bax and Bak genes.

In certain embodiments, the present disclosure provides methods of producing a recombinant polypeptide, comprising reducing apoptotic activity according as described herein (e.g. under the heading “Methods for modulating Bax andbakexpression”), followed by producing the recombinant polypeptide according to a method of disclosure (e.g. as disclosed hereinabove).

### 5.6 Products

The cells, and/or cell lines, and/or methods of the disclosure may be used to produce any product of interest that can be expressed by the cells disclosed herein. The cells, and/or cell lines, and/or methods of the present disclosure may be used for the production of polypeptides, e.g., mammalian polypeptides. Non-limiting examples of such polypeptides include hormones, receptors, fusion proteins including antibody fusion proteins (for e.g., antibody-cytokine fusion proteins), regulatory factors, growth factors, complement system factors, enzymes, clotting factors, anti-clotting factors, kinases, cytokines, CD proteins, interleukins, therapeutic proteins, diagnostic proteins and antibodies. The cells, and/or cell lines, and/or methods of the present disclosure are typically not specific to the molecule, e.g., antibody, that is being produced.

The methods of the present disclosure may be used for the production of antibodies, including therapeutic and diagnostic antibodies or antigen-binding fragments thereof. The antibody produced by cells, cell lines, and/or methods of the disclosure can be, but are not limited to, monospecific antibodies (e.g., antibodies consisting of a single heavy chain sequence and a single light chain sequence, including multimers of such pairings), multispecific antibodies and antigen-binding fragments thereof. For example, but not by way of limitation, the multispecific antibody can be a bispecific antibody, a biepitopic antibody, a T-cell-dependent bispecific antibody (TDB), a Dual Acting FAb (DAF) or antigen-binding fragments thereof.

### Multispecific Antibodies

An antibody may be a multispecific antibody, e.g., a bispecific antibody. “Multispecific antibodies” are monoclonal antibodies that have binding specificities for at least two different sites, i.e., different epitopes on different antigens (i.e., bispecific) or different epitopes on the same antigen (i.e., biepitopic). The multispecific antibody may have three or more binding specificities. Multispecific antibodies can be prepared as full length antibodies or antibody fragments as described herein.

Techniques for making multispecific antibodies include, but are not limited to, recombinant co-expression of two immunoglobulin heavy chain-light chain pairs having different specificities (see Milstein and Cuello, Nature 305: 537 (1983)) and “knob-in-hole” engineering (see, e.g., U.S. Pat. No. 5,731,168, and Atwell et al., J. Mol. Biol. 270:26 (1997)). Multispecific antibodies can also be made by engineering electrostatic steering effects for making antibody Fc-heterodimeric molecules (see, e.g., WO 2009/089004); cross-linking two or more antibodies or fragments (see, e.g., U.S. Pat. No. 4,676,980, and Brennan et al., Science, 229: 81 (1985)); using leucine zippers to produce bi-specific antibodies (see, e.g., Kostelny et al., J. Immunol., 148(5):1547-1553 (1992) and WO 2011/034605); using the common light chain technology for circumventing the light chain mis-pairing problem (see, e.g., WO 98/50431); using “diabody” technology for making bispecific antibody fragments (see, e.g., Hollinger et al., Proc. Natl. Acad. Sci. USA, 90:6444-6448 (1993)); and using single-chain Fv (sFv) dimers (see, e.g., Gruber et al., J. Immunol., 152:5368 (1994)); and preparing trispecific antibodies as described, e.g., in Tutt et al. J. Immunol. 147: 60 (1991).

Engineered antibodies with three or more antigen binding sites, including for example, “Octopus antibodies”, or DVD-Ig are also included herein (see, e.g., WO 2001/77342 and WO 2008/024715). Other non-limiting examples of multispecific antibodies with three or more antigen binding sites can be found in WO 2010/115589, WO 2010/112193, WO 2010/136172, WO 2010/145792 and WO 2013/026831. The bispecific antibody or antigen binding fragment thereof also includes a “Dual Acting FAb” or “DAF” (see, e.g., US 2008/0069820 and WO 2015/095539.

Multispecific antibodies may also be provided in an asymmetric form with a domain crossover in one or more binding arms of the same antigen specificity, i.e., by exchanging the VH/VL domains (see, e.g., WO 2009/080252 and WO 2015/150447), the CH1/CL domains (see, e.g., WO 2009/080253) or the complete Fab arms (see, e.g., WO 2009/080251, WO 2016/016299, also see Schaefer et al, PNAS, 108 (2011) 1187-1191, and Klein at al., MAbs 8 (2016) 1010-20). A multispecific antibody may comprise a cross-Fab fragment. The term “cross-Fab fragment” or “xFab fragment” or “crossover Fab fragment” refers to a Fab fragment, wherein either the variable regions or the constant regions of the heavy and light chain are exchanged. A cross-Fab fragment comprises a polypeptide chain composed of the light chain variable region (VL) and the heavy chain constant region 1 (CH1), and a polypeptide chain composed of the heavy chain variable region (VH) and the light chain constant region (CL). Asymmetrical Fab arms can also be engineered by introducing charged or non-charged amino acid mutations into domain interfaces to direct correct Fab pairing. See, e.g., WO 2016/172485.

Various further molecular formats for multispecific antibodies are known in the art and are included herein (see, e.g., Spiess et al., Mol. Immunol. 67 (2015) 95-106).

A particular type of multispecific antibodies, also included herein, are bispecific antibodies designed to simultaneously bind to a surface antigen on a target cell, e.g., a tumor cell, and to an activating, invariant component of the T cell receptor (TCR) complex, such as CD3, for retargeting of T cells to kill target cells.

Additional non-limiting examples of bispecific antibody formats that can be useful for this purpose include, but are not limited to, the so-called “BiTE” (bispecific T cell engager) molecules wherein two scFv molecules are fused by a flexible linker (see, e.g., WO 2004/106381, WO 2005/061547, WO 2007/042261, and WO 2008/119567, Nagorsen and Bäuerle, Exp Cell Res 317, 1255-1260 (2011)); diabodies (Holliger et al., Prot. Eng. 9, 299-305 (1996)) and derivatives thereof, such as tandem diabodies (“TandAb”; Kipriyanov et al., J Mol Biol 293, 41-56 (1999)); “DART” (dual affinity retargeting) molecules which are based on the diabody format but feature a C-terminal disulfide bridge for additional stabilization (Johnson et al., J Mol Biol 399, 436-449 (2010)), and so-called triomabs, which are whole hybrid mouse/rat IgG molecules (reviewed in Seimetz et al., Cancer Treat. Rev. 36, 458-467 (2010)). Particular T cell bispecific antibody formats included herein are described in WO 2013/026833, WO 2013/026839, WO 2016/020309; Bacac et al., Oncoimmunology 5(8) (2016) e1203498.

### Antibody Fragments

An antibody produced by the cells, and/or cell lines, and/or methods provided herein may be an antibody fragment. For example, but not by way of limitation, the antibody fragment may be a Fab, Fab′, Fab′-SH or F(ab′)2 fragment, in particular a Fab fragment. Papain digestion of intact antibodies produces two identical antigen-binding fragments, called “Fab” fragments containing each the heavy- and light-chain variable domains (VH and VL, respectively) and also the constant domain of the light chain (CL) and the first constant domain of the heavy chain (CH1). The term “Fab fragment” thus refers to an antibody fragment comprising a light chain comprising a VL domain and a CL domain, and a heavy chain fragment comprising a VH domain and a CH1 domain. “Fab′ fragments” differ from Fab fragments by the addition of residues at the carboxy terminus of the CH1 domain including one or more cysteines from the antibody hinge region. Fab′-SH are Fab′ fragments in which the cysteine residue(s) of the constant domains bear a free thiol group. Pepsin treatment yields an F(ab′)2 fragment that has two antigen-binding sites (two Fab fragments) and a part of the Fc region. For discussion of Fab and F(ab′)2 fragments comprising salvage receptor binding epitope residues and having increased in vivo half-life, see U.S. Pat. No. 5,869,046.

The antibody fragment may be a diabody, a triabody or a tetrabody. “Diabodies” are antibody fragments with two antigen-binding sites that can be bivalent or bispecific. See, for example, EP 404,097; WO 1993/01161; Hudson et al., Nat. Med. 9:129-134 (2003); and Hollinger et al., Proc. Natl. Acad. Sci. USA 90: 6444-6448 (1993). Triabodies and tetrabodies are also described in Hudson et al., Nat. Med. 9:129-134 (2003).

The antibody fragment may be a single chain Fab fragment. A “single chain Fab fragment” or “scFab” is a polypeptide consisting of an antibody heavy chain variable domain (VH), an antibody heavy chain constant domain 1 (CH1), an antibody light chain variable domain (VL), an antibody light chain constant domain (CL) and a linker, wherein said antibody domains and said linker have one of the following orders in N-terminal to C-terminal direction: a) VH-CH1-linker-VL-CL, b) VL-CL-linker-VH-CH1, c) VH-CL-linker-VL-CH1 or d) VL-CH1-linker-VH-CL. In particular, said linker may be a polypeptide of at least 30 amino acids, preferably between 32 and 50 amino acids. Said single chain Fab fragments are stabilized via the natural disulfide bond between the CL domain and the CH1 domain. In addition, these single chain Fab fragments might be further stabilized by generation of interchain disulfide bonds via insertion of cysteine residues (e.g., position 44 in the variable heavy chain and position 100 in the variable light chain according to Kabat numbering).

The antibody fragment may be a single-chain variable fragment (scFv). A “single-chain variable fragment” or “scFv” is a fusion protein of the variable domains of the heavy (VH) and light chains (VL) of an antibody, connected by a linker. In particular, the linker may be a short polypeptide of 10 to 25 amino acids and is usually rich in glycine for flexibility, as well as serine or threonine for solubility, and can either connect the N-terminus of the VH with the C-terminus of the VL, or vice versa. This protein retains the specificity of the original antibody, despite removal of the constant regions and the introduction of the linker. For a review of scFv fragments, see, e.g., Plückthun, in The Pharmacology of Monoclonal Antibodies, vol. 113, Rosenburg and Moore eds., (Springer-Verlag, New York), pp. 269-315 (1994); see also WO 93/16185; and U.S. Pat. Nos. 5,571,894 and 5,587,458.

The antibody fragment may be a single-domain antibody. “Single-domain antibodies” are antibody fragments comprising all or a portion of the heavy chain variable domain or all or a portion of the light chain variable domain of an antibody. A single-domain antibody may be a human single-domain antibody (Domantis, Inc., Waltham, MA; see, e.g., U.S. Pat. No. 6,248,516 B1).

In certain aspects, an antibody fusion protein produced by the cells and methods provided herein is an antibody-cytokine fusion protein. While such antibody-cytokine fusion proteins can comprise full length antibodies, the antibody of the antibody-cytokine fusion protein is, in certain embodiments, an antibody fragment, e.g., a single-chain variable fragment (scFv), a diabodies, aFab fragment, or a small immunoprotein (SIP). In certain embodiments, the cytokine can be fused to the N-terminus or the C-terminus of the antibody. In certain embodiments, the cytokine of the antibody-cytokine fusion protein consists of multiple subunits. In certain embodiments, the subunits of the cytokine are the same (homomeric). In certain embodiments, the subunits of the cytokine are the distinct (heterometic). In certain embodiments, the subunits of the cytokine are fused to the same antibody. In certain embodiments, the subunits of the cytokine are fused to a different antibody. For a review of antibody-cytokine fusion protein, see, e.g., Murer et al., N Biotechnol., 52: 42-53 (2019).

Antibody fragments may be made by various techniques, including but not limited to proteolytic digestion of an intact antibody.

### Chimeric and Humanized Antibodies

An antibody produced by the cells, and/or cell lines, and/or methods provided herein may be a chimeric antibody. Certain chimeric antibodies are described, e.g., in U.S. Pat. No. 4,816,567; and Morrison et al., Proc. Natl. Acad. Sci. USA, 81:6851-6855 (1984)). In one example, a chimeric antibody comprises a non-human variable region (e.g., a variable region derived from a mouse, rat, hamster, rabbit, or non-human primate, such as a monkey) and a human constant region. In a further example, a chimeric antibody is a “class switched” antibody in which the class or subclass has been changed from that of the parent antibody. Chimeric antibodies include antigen-binding fragments thereof.

A chimeric antibody may be a humanized antibody. Typically, a non-human antibody is humanized to reduce immunogenicity to humans, while retaining the specificity and affinity of the parental non-human antibody. Generally, a humanized antibody comprises one or more variable domains in which the CDRs (or portions thereof) are derived from a non-human antibody, and FRs (or portions thereof) are derived from human antibody sequences. A humanized antibody optionally will also comprise at least a portion of a human constant region. In certain embodiments, some FR residues in a humanized antibody are substituted with corresponding residues from a non-human antibody (e.g., the antibody from which the CDR residues are derived), e.g., to restore or improve antibody specificity or affinity.

Humanized antibodies and methods of making them are reviewed, e.g., in Almagro and Fransson, Front. Biosci. 13:1619-1633 (2008), and are further described, e.g., in Riechmann et al., Nature 332:323-329 (1988); Queen et al., Proc. Nat'l Acad. Sci. USA 86:10029-10033 (1989); U.S. Pat. Nos. 5,821,337, 7,527,791, 6,982,321, and 7,087,409; Kashmiri et al., Methods 36:25-34 (2005) (describing specificity determining region (SDR) grafting); Padlan, Mol. Immunol. 28:489-498 (1991) (describing “resurfacing”); Dall'Acqua et al., Methods 36:43-60 (2005) (describing “FR shuffling”); and Osbourn et al., Methods 36:61-68 (2005) and Klimka et al., Br. J. Cancer, 83:252-260 (2000) (describing the “guided selection” approach to FR shuffling).

Human framework regions that can be used for humanization include but are not limited to: framework regions selected using the “best-fit” method (see, e.g., Sims et al. J. Immunol. 151:2296 (1993)); framework regions derived from the consensus sequence of human antibodies of a particular subgroup of light or heavy chain variable regions (see, e.g., Carter et al. Proc. Natl. Acad. Sci. USA, 89:4285 (1992); and Presta et al. J. Immunol., 151:2623 (1993)); human mature (somatically mutated) framework regions or human germline framework regions (see, e.g., Almagro and Fransson, Front. Biosci. 13:1619-1633 (2008)); and framework regions derived from screening FR libraries (see, e.g., Baca et al., J. Biol. Chem. 272:10678-10684 (1997) and Rosok et al., J. Biol. Chem. 271:22611-22618 (1996)).

### Human Antibodies

An antibody produced by the cells, and/or cell lines, and/or methods provided herein may be a human antibody. Human antibodies can be produced using various techniques known in the art. Human antibodies are described generally in van Dijk and van de Winkel, Curr. Opin. Pharmacol. 5: 368-74 (2001) and Lonberg, Curr. Opin. Immunol. 20:450-459 (2008).

Human antibodies may be prepared by administering an immunogen to a transgenic animal that has been modified to produce intact human antibodies or intact antibodies with human variable regions in response to antigenic challenge. Such animals typically contain all or a portion of the human immunoglobulin loci, which replace the endogenous immunoglobulin loci, or which are present extrachromosomally or integrated randomly into the animal's chromosomes. In such transgenic mice, the endogenous immunoglobulin loci have generally been inactivated. For review of methods for obtaining human antibodies from transgenic animals, see Lonberg, Nat. Biotech. 23:1117-1125 (2005). See also, e.g., U.S. Pat. Nos. 6,075,181 and 6,150,584 describing XENOMOUSE™ technology; U.S. Pat. No. 5,770,429 describing HUMAB® technology; U.S. Pat. No. 7,041,870 describing K-M MOUSE® technology, and U.S. Patent Application Publication No. US 2007/0061900, describing VELOCIMOUSE® technology). Human variable regions from intact antibodies generated by such animals can be further modified, e.g., by combining with a different human constant region.

Human antibodies may also be made by hybridoma-based methods. Human myeloma and mouse-human heteromyeloma cell lines for the production of human monoclonal antibodies have been described. (See, e.g., Kozbor J. Immunol., 133: 3001 (1984); Brodeur et al., Monoclonal Antibody Production Techniques and Applications, pp. 51-63 (Marcel Dekker, Inc., New York, 1987); and Boerner et al., J. Immunol., 147: 86 (1991).) Human antibodies generated via human B-cell hybridoma technology are also described in Li et al., Proc. Natl. Acad. Sci. USA, 103:3557-3562 (2006). Additional methods include those described, for example, in U.S. Pat. No. 7,189,826 (describing production of monoclonal human IgM antibodies from hybridoma cell lines) and Ni, Xiandai Mianyixue, 26(4):265-268 (2006) (describing human-human hybridomas). Human hybridoma technology (Trioma technology) is also described in Vollmers and Brandlein, Histology and Histopathology, 20(3):927-937 (2005) and Vollmers and Brandlein, Methods and Findings in Experimental and Clinical Pharmacology, 27(3):185-91 (2005).

### Target Molecules

Non-limiting examples of molecules that may be targeted by an antibody produced by the cells and methods disclosed herein include soluble serum proteins and their receptors and other membrane bound proteins (e.g., adhesins). In certain examples, an antibody produced by the cells and methods disclosed herein is capable of binding to one, two or more cytokines, cytokine-related proteins, and cytokine receptors selected from the group consisting of 8MPI, 8MP2, 8MP38 (GDFIO), 8MP4, 8MP6, 8MP8, CSFI (M-CSF), CSF2 (GM-CSF), CSF3 (G-CSF), EPO, FGF1 (αFGF), FGF2 (βFGF), FGF3 (int-2), FGF4 (HST), FGF5, FGF6 (HST-2), FGF7 (KGF), FGF9, FGF1 0, FGF11, FGF12, FGF12B, FGF14, FGF16, FGF17, FGF19, FGF20, FGF21, FGF23, IGF1, IGF2, IFNA1, IFNA2, IFNA4, IFNA5, IFNA6, IFNA7, IFN81, IFNG, IFNWI, FEL1, FEL1 (EPSELON), FEL1 (ZETA), IL 1A, IL 1B, IL2, IL3, IL4, IL5, IL6, IL7, IL8, IL9, IL10, IL 11, IL 12A, IL 12B, IL 13, IL 14, IL 15, IL 16, IL 17, IL 17B, IL 18, IL 19, IL20, IL22, IL23, IL24, IL25, IL26, IL27, IL28A, IL28B, IL29, IL30, PDGFA, PDGFB, TGFA, TGFB1, TGFB2, TGFBb3, LTA (TNF-β), LTB, TNF (TNF-α), TNFSF4 (OX40 ligand), TNFSF5 (CD40 ligand), TNFSF6 (FasL), TNFSF7 (CD27 ligand), TNFSF8 (CD30 ligand), TNFSF9 (4-1 BB ligand), TNFSF10 (TRAIL), TNFSF11 (TRANCE), TNFSF12 (APO3L), TNFSF13 (April), TNFSF13B, TNFSF14 (HVEM-L), TNFSF15 (VEGI), TNFSF18, HGF (VEGFD), VEGF, VEGFB, VEGFC, IL1R1, IL1R2, IL1RL1, IL1RL2, IL2RA, IL2RB, IL2RG, IL3RA, IL4R, IL5RA, IL6R, IL7R, IL8RA, IL8RB, IL9R, IL10RA, IL10RB, IL 11RA, IL12RB1, IL12RB2, IL13RA1, IL13RA2, IL15RA, IL17R, IL18R1, IL20RA, IL21R, IL22R, IL1HY1, IL1RAP, IL1RAPL1, IL1RAPL2, IL1RN, IL6ST, IL18BP, IL18RAP, IL22RA2, AIF1, HGF, LEP (leptin), PTN, and THPO.

An antibody produced by cells and methods disclosed herein may be capable of binding to a chemokine, chemokine receptor, or a chemokine-related protein selected from the group consisting of CCLI (1-309), CCL2 (MCP-1/MCAF), CCL3 (MIP-Iα), CCL4 (MIP-Iβ), CCL5 (RANTES), CCL7 (MCP-3), CCL8 (mcp-2), CCL11 (eotaxin), CCL 13 (MCP-4), CCL 15 (MIP-Iδ), CCL 16 (HCC-4), CCL 17 (TARC), CCL 18 (PARC), CCL 19 (MDP-3b), CCL20 (MIP-3α), CCL21 (SLC/exodus-2), CCL22 (MDC/STC-1), CCL23 (MPIF-1), CCL24 (MPIF-2/eotaxin-2), CCL25 (TECK), CCL26 (eotaxin-3), CCL27 (CTACK/ILC), CCL28, CXCLI (GROI), CXCL2 (GR02), CXCL3 (GR03), CXCL5 (ENA-78), CXCL6 (GCP-2), CXCL9 (MIG), CXCL 10 (IP 10), CXCL 11 (1-TAC), CXCL 12 (SDFI), CXCL 13, CXCL 14, CXCL 16, PF4 (CXCL4), PPBP (CXCL7), CX3CL 1 (SCYDI), SCYEI, XCLI (lymphotactin), XCL2 (SCM-Iβ), BLRI (MDR15), CCBP2 (D6/JAB61), CCRI (CKRI/HM145), CCR2 (mcp-IRB IRA), CCR3 (CKR3/CMKBR3), CCR4, CCR5 (CMKBR5/ChemR13), CCR6 (CMKBR6/CKR-L3/STRL22/DRY6), CCR7 (CKR7/EBII), CCR8 (CMKBR8/TER1/CKR-L1), CCR9 (GPR-9-6), CCRL1 (VSHK1), CCRL2 (L-CCR), XCR1 (GPR5/CCXCR1), CMKLR1, CMKOR1 (RDC1), CX3CR1 (V28), CXCR4, GPR2 (CCR10), GPR31, GPR81 (FKSG80), CXCR3 (GPR9/CKR-L2), CXCR6 (TYMSTR/STRL33/Bonzo), HM74, IL8RA (IL8Rα), IL8RB (IL8Rβ), LTB4R (GPR16), TCP10, CKLFSF2, CKLFSF3, CKLFSF4, CKLFSF5, CKLFSF6, CKLFSF7, CKLFSF8, BDNF, C5, C5R1, CSF3, GRCC10 (C10), EPO, FY (DARC), GDF5, HDF1, HDF1α, DL8, PRL, RGS3, RGS13, SDF2, SLIT2, TLR2, TLR4, TREM1, TREM2, and VHL.

In certain examples, an antibody produced by methods disclosed herein (e.g., a multispecific antibody such as a bispecific antibody) is capable of binding to one or more target molecules selected from the following: 0772P (CA125, MUC16) (i.e., ovarian cancer antigen), ABCF1; ACVR1; ACVR1B; ACVR2; ACVR2B; ACVRL1; ADORA2A; Aggrecan; AGR2; AICDA; AIF1; AIG1; AKAP1; AKAP2; AMH; AMHR2; amyloid beta; ANGPTL; ANGPT2; ANGPTL3; ANGPTL4; ANPEP; APC; APOC1; AR; ASLG659; ASPHD1 (aspartate beta-hydroxylase domain containing 1; LOC253982); AZGP1 (zinc-a-glycoprotein); B7.1; B7.2; BAD; BAFF-R (B cell-activating factor receptor, BLyS receptor 3, BR3; BAG1; BAI1; BCL2; BCL6; BDNF; BLNK; BLRI (MDR15); BMP1; BMP2; BMP3B (GDF10); BMP4; BMP6; BMP8; BMPR1A; BMPR1B (bone morphogenic protein receptor-type IB); BMPR2; BPAG1 (plectin); BRCA1; Brevican; C19orf10 (IL27w); C3; C4A; C5; C5R1; CANT1; CASP1; CASP4; CAV1; CCBP2 (D6/JAB61); CCL1 (1-309); CCL11 (eotaxin); CCL13 (MCP-4); CCL15 (MIP1δ); CCL16 (HCC-4); CCL17 (TARC); CCL18 (PARC); CCL19 (MIP-30); CCL2 (MCP-1); MCAF; CCL20 (MIP-3a); CCL21 (MTP-2); SLC; exodus-2; CCL22 (MDC/STC-1); CCL23 (MPIF-1); CCL24 (MPIF-2/eotaxin-2); CCL25 (TECK); CCL26 (eotaxin-3); CCL27 (CTACK/ILC); CCL28; CCL3 (MTP-Iα); CCL4 (MDP-Iβ); CCL5(RANTES); CCL7 (MCP-3); CCL8 (mcp-2); CCNA1; CCNA2; CCND1; CCNE1; CCNE2; CCR1 (CKRI/HM145); CCR2 (mcp-IRP/RA); CCR3 (CKR/CMKBR3); CCR4; CCR5 (CMKBR5/ChemR13); CCR6 (CMKBR6/CKR-L3/STRL22/DRY6); CCR7 (CKBR7/EBI1); CCR8 (CMKBR8/TER1/CKR-L1); CCR9 (GPR-9-6); CCRL1 (VSHK1); CCRL2 (L-CCR); CD164; CD19; CD1C; CD20; CD200; CD22 (B-cell receptor CD22-B isoform); CD24; CD28; CD3; CD37; CD38; CD3E; CD3G; CD3Z; CD4; CD40; CD40L; CD44; CD45RB; CD52; CD69; CD72; CD74; CD79A (CD79a, immunoglobulin-associated alpha, a B cell-specific protein); CD79B; CDS; CD80; CD81; CD83; CD86; CDH1 (E-cadherin); CDH10; CDH12; CDH13; CDH18; CDH19; CDH20; CDH5; CDH7; CDH8; CDH9; CDK2; CDK3; CDK4; CDK5; CDK6; CDK7; CDK9; CDKN1A (p21/WAF1/Cip1); CDKN1B (p27/Kip1); CDKN1C; CDKN2A (P16INK4a); CDKN2B; CDKN2C; CDKN3; CEBPB; CER1; CHGA; CHGB; Chitinase; CHST10; CKLFSF2; CKLFSF3; CKLFSF4; CKLFSF5; CKLFSF6; CKLFSF7; CKLFSF8; CLDN3; CLDN7 (claudin-7); CLL-1 (CLEC12A, MICL, and DCAL2); CLN3; CLU (clusterin); CMKLR1; CMKOR1 (RDC1); CNR1; COL 18A1; COL1A1; COL4A3; COL6A1; complement factor D; CR2; CRP; CRIPTO (CR, CR1, CRGF, CRIPTO, TDGF1, teratocarcinoma-derived growth factor); CSFI (M-CSF); CSF2 (GM-CSF); CSF3 (GCSF); CTLA4; CTNNB1 (b-catenin); CTSB (cathepsin B); CX3CL1 (SCYDI); CX3CR1 (V28); CXCL1 (GRO1); CXCL10 (IP-10); CXCL11 (I-TAC/IP-9); CXCL12 (SDF1); CXCL13; CXCL14; CXCL16; CXCL2 (GRO2); CXCL3 (GRO3); CXCL5 (ENA-78/LIX); CXCL6 (GCP-2); CXCL9 (MIG); CXCR3 (GPR9/CKR-L2); CXCR4; CXCR5 (Burkitt's lymphoma receptor 1, a G protein-coupled receptor); CXCR6 (TYMSTR/STRL33/Bonzo); CYB5; CYC1; CYSLTR1; DAB2IP; DES; DKFZp451J0118; DNCLI; DPP4; E16 (LAT1, SLC7A5); E2F1; ECGF1; EDG1; EFNA1; EFNA3; EFNB2; EGF; EGFR; ELAC2; ENG; ENO1; ENO2; ENO3; EPHB4; EphB2R; EPO; ERBB2 (Her-2); EREG; ERK8; ESR1; ESR2; ETBR (Endothelin type B receptor); F3 (TF); FADD; FasL; FASN; FCER1A; FCER2; FCGR3A; FcRH1 (Fc receptor-like protein 1); FcRH2 (IFGP4, IRTA4, SPAP1A (SH2 domain containing phosphatase anchor protein 1a), SPAP1B, SPAP1C); FGF; FGF1 (αFGF); FGF10; FGF11; FGF12; FGF12B; FGF13; FGF14; FGF16; FGF17; FGF18; FGF19; FGF2 (bFGF); FGF20; FGF21; FGF22; FGF23; FGF3 (int-2); FGF4 (HST); FGF5; FGF6 (HST-2); FGF7 (KGF); FGF8; FGF9; FGFR; FGFR3; FIGF (VEGFD); FEL1 (EPSILON); FIL1 (ZETA); FLJ12584; FLJ25530; FLRTI (fibronectin); FLT1; FOS; FOSL1 (FRA-1); FY (DARC); GABRP (GABAa); GAGEB1; GAGEC1; GALNAC4S-6ST; GATA3; GDF5; GDNF-Ra1 (GDNF family receptor alpha 1; GFRA1; GDNFR; GDNFRA; RETL1; TRNR1; RET1L; GDNFR-alpha1; GFR-ALPHA-1); GEDA; GFI1; GGT1; GM-CSF; GNASI; GNRHI; GPR2 (CCR10); GPR19 (G protein-coupled receptor 19; Mm.4787); GPR31; GPR44; GPR54 (KISS1 receptor; KISS1R; GPR54; HOT7T175; AXOR12); GPR81 (FKSG80); GPR172A (G protein-coupled receptor 172A; GPCR41; FLJ11856; D15Ertd747e); GRCCIO (C10); GRP; GSN (Gelsolin); GSTP1; HAVCR2; HDAC4; HDAC5; HDAC7A; HDAC9; HGF; HIF1A; HOP1; histamine and histamine receptors; HLA-A; HLA-DOB (Beta subunit of MHC class II molecule (Ia antigen); HLA-DRA; HM74; HMOXI; HUMCYT2A; ICEBERG; ICOSL; 1D2; IFN-a; IFNA1; IFNA2; IFNA4; IFNA5; IFNA6; IFNA7; IFNB1; IFNgamma; DFNW1; IGBP1; IGF1; IGF1R; IGF2; IGFBP2; IGFBP3; IGFBP6; IL-1; IL10; IL10RA; IL10RB; IL11; IL11RA; IL-12; IL12A; IL12B; IL12RB1; IL12RB2; IL13; IL13RA1; IL13RA2; IL14; IL15; IL15RA; IL16; IL17; IL17B; IL17C; IL17R; IL18; IL18BP; IL18R1; IL18RAP; IL19; IL1A; IL1B; ILIF10; IL1F5; IL1F6; IL1F7; IL1F8; IL1F9; IL1HY1; IL1R1; IL1R2; IL1RAP; IL1RAPL1; IL1RAPL2; IL1RL1; IL1RL2, ILIRN; IL2; IL20; IL20Ra; IL21 R; IL22; IL-22c; IL22R; IL22RA2; IL23; IL24; IL25; IL26; IL27; IL28A; IL28B; IL29; IL2RA; IL2RB; IL2RG; IL3; IL30; IL3RA; IL4; IL4R; IL5; IL5RA; IL6; IL6R; IL6ST (glycoprotein 130); influenza A; influenza B; EL7; EL7R; EL8; IL8RA; DL8RB; IL8RB; DL9; DL9R; DLK; INHA; INHBA; INSL3; INSL4; IRAK1; IRTA2 (Immunoglobulin superfamily receptor translocation associated 2); ERAK2; ITGA1; ITGA2; ITGA3; ITGA6 (a6 integrin); ITGAV; ITGB3; ITGB4 (b4 integrin); α4β7 and αEβ7 integrin heterodimers; JAG1; JAK1; JAK3; JUN; K6HF; KAI1; KDR; KITLG; KLF5 (GC Box BP); KLF6; KLKIO; KLK12; KLK13; KLK14; KLK15; KLK3; KLK4; KLK5; KLK6; KLK9; KRT1; KRT19 (Keratin 19); KRT2A; KHTHB6 (hair-specific type H keratin); LAMAS; LEP (leptin); LGR5 (leucine-rich repeat-containing G protein-coupled receptor 5; GPR49, GPR67); Lingo-p75; Lingo-Troy; LPS; LTA (TNF-b); LTB; LTB4R (GPR16); LTB4R2; LTBR; LY64 (Lymphocyte antigen 64 (RP105), type I membrane protein of the leucine rich repeat (LRR) family); Ly6E (lymphocyte antigen 6 complex, locus E; Ly67,RIG-E,SCA-2,TSA-1); Ly6G6D (lymphocyte antigen 6 complex, locus G6D; Ly6-D, MEGT1); LY6K (lymphocyte antigen 6 complex, locus K; LY6K; HSJ001348; FLJ35226); MACMARCKS; MAG or OMgp; MAP2K7 (c-Jun); MDK; MDP; MIB1; midkine; MEF; MIP-2; MKI67; (Ki-67); MMP2; MMP9; MPF (MPF, MSLN, SMR, megakaryocyte potentiating factor, mesothelin); MS4A1; MSG783 (RNF124, hypothetical protein FLJ20315); MSMB; MT3 (metallothionectin-111); MTSS1; MUC1 (mucin); MYC; MY088; Napi3b (also known as NaPi2b) (NAPI-3B, NPTIIb, SLC34A2, solute carrier family 34 (sodium phosphate), member 2, type II sodium-dependent phosphate transporter 3b); NCA; NCK2; neurocan; NFKB1; NFKB2; NGFB (NGF); NGFR; NgR-Lingo; NgR-Nogo66 (Nogo); NgR-p75; NgR-Troy; NME1 (NM23A); NOX5; NPPB; NR0B1; NR0B2; NR1D1; NR1D2; NR1H2; NR1H3; NR1H4; NR112; NR113; NR2C1; NR2C2; NR2E1; NR2E3; NR2F1; NR2F2; NR2F6; NR3C1; NR3C2; NR4A1; NR4A2; NR4A3; NR5A1; NR5A2; NR6A1; NRP1; NRP2; NT5E; NTN4; ODZI; OPRD1; OX40; P2RX7; P2X5 (Purinergic receptor P2X ligand-gated ion channel 5); PAP; PART1; PATE; PAWR; PCA3; PCNA; PD-L1; PD-L2; PD-1; POGFA; POGFB; PECAM1; PF4 (CXCL4); PGF; PGR; phosphacan; PIAS2; PIK3CG; PLAU (uPA); PLG; PLXDC1; PMEL17 (silver homolog; SILV; D12S53E; PMEL17; SI; SIL); PPBP (CXCL7); PPID; PRI; PRKCQ; PRKDI; PRL; PROC; PROK2; PSAP; PSCA hlg (2700050C12Rik, C530008016Rik, RIKEN cDNA 2700050C12, RIKEN cDNA 2700050C12 gene); PTAFR; PTEN; PTGS2 (COX-2); PTN; RAC2 (p21 Rac2); RARB; RET (ret proto-oncogene; MEN2A; HSCR1; MEN2B; MTC1; PTC; CDHF12; Hs.168114; RET51; RET-ELE1); RGSI; RGS13; RGS3; RNF110 (ZNF144); ROBO2; S100A2; SCGB1D2 (lipophilin B); SCGB2A1 (mammaglobin2); SCGB2A2 (mammaglobin 1); SCYEI (endothelial Monocyte-activating cytokine); SDF2; Sema 5b (FLJ10372, KIAA1445, Mm.42015, SEMA5B, SEMAG, Semaphorin 5b Hlog, sema domain, seven thrombospondin repeats (type 1 and type 1-like), transmembrane domain (TM) and short cytoplasmic domain, (semaphorin) 5B); SERPINA1; SERPINA3; SERPINB5 (maspin); SERPINE1(PAI-1); SERPDMF1; SHBG; SLA2; SLC2A2; SLC33A1; SLC43A1; SLIT2; SPPI; SPRR1B (Sprl); ST6GAL1; STABI; STAT6; STEAP (six transmembrane epithelial antigen of prostate); STEAP2 (HGNC_8639, IPCA-1, PCANAP1, STAMP1, STEAP2, STMP, prostate cancer associated gene 1, prostate cancer associated protein 1, six transmembrane epithelial antigen of prostate 2, six transmembrane prostate protein); TB4R2; TBX21; TCPIO; TOGFI; TEK; TENB2 (putative transmembrane proteoglycan); TGFA; TGFBI; TGFBIII; TGFB2; TGFB3; TGFBI; TGFBRI; TGFBR2; TGFBR3; THIL; THBSI (thrombospondin-1); THBS2; THBS4; THPO; TIE (Tie-1); TMP3; tissue factor; TLR1; TLR2; TLR3; TLR4; TLR5; TLR6; TLR7; TLR8; TLR9; TLR10; TMEFF1 (transmembrane protein with EGF-like and two follistatin-like domains 1; Tomoregulin-1); TMEM46 (shisa homolog 2); TNF; TNF-a; TNFAEP2 (B94); TNFAIP3; TNFRSFIIA; TNFRSF1A; TNFRSF1B; TNFRSF21; TNFRSF5; TNFRSF6 (Fas); TNFRSF7; TNFRSF8; TNFRSF9; TNFSF10 (TRAIL); TNFSF11 (TRANCE); TNFSF12 (AP03L); TNFSF13 (April); TNFSF13B; TNFSF14 (HVEM-L); TNFSF15 (VEGI); TNFSF18; TNFSF4 (OX40 ligand); TNFSF5 (CD40 ligand); TNFSF6 (FasL); TNFSF7 (CD27 ligand); TNFSFS (CD30 ligand); TNFSF9 (4-1 BB ligand); TOLLIP; Toll-like receptors; TOP2A (topoisomerase Ea); TP53; TPM1; TPM2; TRADD; TMEM118 (ring finger protein, transmembrane 2; RNFT2; FLJ14627); TRAF1; TRAF2; TRAF3; TRAF4; TRAF5; TRAF6; TREM1; TREM2; TrpM4 (BR22450, FLJ20041, TRPM4, TRPM4B, transient receptor potential cation channel, subfamily M, member 4); TRPC6; TSLP; TWEAK; Tyrosinase (TYR; OCAIA; OCA1A; tyrosinase; SHEP3); VEGF; VEGFB; VEGFC; versican; VHL C5; VLA-4; XCL1 (lymphotactin); XCL2 (SCM-1b); XCRI (GPR5/CCXCRI); YY1; and ZFPM2.

In certain examples, an antibody produced by the cells and methods disclosed herein is capable of binding to CD proteins such as CD3, CD4, CD5, CD16, CD19, CD20, CD21 (CR2 (Complement receptor 2) or C3DR (C3d/Epstein Barr virus receptor) or Hs.73792); CD33; CD34; CD64; CD72 (B-cell differentiation antigen CD72, Lyb-2); CD79b (CD79B, CD79β, IGb (immunoglobulin-associated beta), B29); CD200 members of the ErbB receptor family such as the EGF receptor, HER2, HER3, or HER4 receptor; cell adhesion molecules such as LFA-1, Mac1, p150.95, VLA-4, ICAM-1, VCAM, alpha4/beta7 integrin, and alphav/beta3 integrin including either alpha or beta subunits thereof (e.g., anti-CD11a, anti-CD18, or anti-CD11b antibodies); growth factors such as VEGF-A, VEGF-C; tissue factor (TF); alpha interferon (alphaIFN); TNFalpha, an interleukin, such as IL-1 beta, IL-3, IL-4, IL-5, IL-6, IL-8, IL-9, IL-13, IL 17 AF, IL-1S, IL-13R alpha1, IL13R alpha2, IL-4R, IL-5R, IL-9R, IgE; blood group antigens; flk2/flt3 receptor; obesity (OB) receptor; mpl receptor; CTLA-4; RANKL, RANK, RSV F protein, protein C etc.

In certain examples, the cells, cell lines and methods provided herein can be used to produce an antibody (or a multispecific antibody, such as a bispecific antibody) that specifically binds to complement protein C5 (e.g., an anti-C5 agonist antibody that specifically binds to human C5). The anti-C5 antibody may comprise 1, 2, 3, 4, 5 or 6 CDRs selected from (a) a heavy chain variable region CDR1 comprising the amino acid sequence of SSYYMA (SEQ ID NO:1); (b) a heavy chain variable region CDR2 comprising the amino acid sequence of AIFTGSGAEYKAEWAKG (SEQ ID NO:26); (c) a heavy chain variable region CDR3 comprising the amino acid sequence of DAGYDYPTHAMHY (SEQ ID NO: 27); (d) a light chain variable region CDR1 comprising the amino acid sequence of RASQGISSSLA (SEQ ID NO: 28); (e) a light chain variable region CDR2 comprising the amino acid sequence of GASETES (SEQ ID NO: 29); and (f) a light chain variable region CDR3 comprising the amino acid sequence of QNTKVGSSYGNT (SEQ ID NO: 30). For example, the anti-C5 antibody may comprise a heavy chain variable domain (VH) sequence comprising one, two or three CDRs selected from: (a) a heavy chain variable region CDR1 comprising the amino acid sequence of (SSYYMA (SEQ ID NO: 1); (b) a heavy chain variable region CDR2 comprising the amino acid sequence of AIFTGSGAEYKAEWAKG (SEQ ID NO: 26); (c) a heavy chain variable region CDR3 comprising the amino acid sequence of DAGYDYPTHAMHY (SEQ ID NO: 27); and/or a light chain variable domain (VL) sequence comprising one, two or three CDRs selected from (d) a light chain variable region CDR1 comprising the amino acid sequence of RASQGISSSLA (SEQ ID NO: 28); (e) a light chain variable region CDR2 comprising the amino acid sequence of GASETES (SEQ ID NO: 29); and (f) a light chain variable region CDR3 comprising the amino acid sequence of QNTKVGSSYGNT (SEQ ID NO: 30). The sequences of CDR1, CDR2 and CDR3 of the heavy chain variable region and CDR1, CDR2 and CDR3 of the light chain variable region above are disclosed in US 2016/0176954 as SEQ ID NO: 117, SEQ ID NO: 118, SEQ ID NO: 121, SEQ ID NO: 122, SEQ ID NO: 123, and SEQ ID NO: 125, respectively. (See Tables 7 and 8 in US 2016/0176954.)

In certain examples, the anti-C5 antibody comprises the VH and VL sequences QVQLVESGGG LVQPGRSLRL SCAASGFTVH SSYYMAWVRQ APGKGLEWVG AIFTGSGAEY KAEWAKGRVT ISKDTSKNQV VLTMTNMDPV DTATYYCASD AGYDYPTHAM HYWGQGTLVT VSS (SEQ ID NO: 31) and DIQMTQSPSS LSASVGDRVT ITCRASQGIS SSLAWYQQKP GKAPKLLIYG ASETESGVPS RFSGSGSGTD FTLTISSLQP EDFATYYCQN TKVGSSYGNT FGGGTKVEIK (SEQ ID NO: 32), respectively, including post-translational modifications of those sequences. The VH and VL sequences above are disclosed in US 2016/0176954 as SEQ ID NO: 106 and SEQ ID NO: 111, respectively. (See Tables 7 and 8 in US 2016/0176954.) The anti-C5 antibody may be 305L015 (see US 2016/0176954).

In certain examples, an antibody produced by methods disclosed herein is capable of binding to OX40 (e.g., an anti-OX40 agonist antibody that specifically binds to human OX40). The anti-OX40 antibody may comprise 1, 2, 3, 4, 5 or 6 CDRs selected from (a) a heavy chain variable region CDR1 comprising the amino acid sequence of DSYMS (SEQ ID NO: 2); (b) a heavy chain variable region CDR2 comprising the amino acid sequence of DMYPDNGDSSYNQKFRE (SEQ ID NO: 3); (c) a heavy chain variable region CDR3 comprising the amino acid sequence of APRWYFSV (SEQ ID NO: 4); (d) a light chain variable region CDR1 comprising the amino acid sequence of RASQDISNYLN (SEQ ID NO: 5); (e) a light chain variable region CDR2 comprising the amino acid sequence of YTSRLRS (SEQ ID NO: 6); and (f) a light chain variable region CDR3 comprising the amino acid sequence of QQGHTLPPT (SEQ ID NO: 7). For example, the anti-OX40 antibody may comprise a heavy chain variable domain (VH) sequence comprising one, two or three CDRs selected from: (a) a heavy chain variable region CDR1 comprising the amino acid sequence of DSYMS (SEQ ID NO: 2); (b) a heavy chain variable region CDR2 comprising the amino acid sequence of DMYPDNGDSSYNQKFRE (SEQ ID NO: 3); and (c) a heavy chain variable region CDR3 comprising the amino acid sequence of APRWYFSV (SEQ ID NO: 4) and/or a light chain variable domain (VL) sequence comprising one, two or three CDRs selected from (a) a light chain variable region CDR1 comprising the amino acid sequence of RASQDISNYLN (SEQ ID NO: 5); (b) a light chain variable region CDR2 comprising the amino acid sequence of YTSRLRS (SEQ ID NO: 6); and (c) a light chain variable region CDR3 comprising the amino acid sequence of QQGHTLPPT (SEQ ID NO: 7). The anti-OX40 antibody may comprise the VH and VL sequences EVQLVQSGAE VKKPGASVKV SCKASGYTFT DSYMSWVRQA PGQGLEWIGD MYPDNGDSSY NQKFRERVTI TRDTSTSTAY LELSSLRSED TAVYYCVLAP RWYFSVWGQG TLVTVSS (SEQ ID NO: 8) and DIQMTQSPSS LSASVGDRVT ITCRASQDIS NYLNWYQQKP GKAPKLLIYY TSRLRSGVPS RFSGSGSGTD FTLTISSLQP EDFATYYCQQ GHTLPPTFGQ GTKVEIK (SEQ ID NO: 9), respectively, including post-translational modifications of those sequences.

In certain examples, the anti-OX40 antibody comprises 1, 2, 3, 4, 5 or 6 CDRs selected from (a) a heavy chain variable region CDR1 comprising the amino acid sequence of NYLIE (SEQ ID NO: 10); (b) a heavy chain variable region CDR2 comprising the amino acid sequence of VINPGSGDTYYSEKFKG (SEQ ID NO: 11); (c) a heavy chain variable region CDR3 comprising the amino acid sequence of DRLDY (SEQ ID NO: 12); (d) a light chain variable region CDR1 comprising the amino acid sequence of HASQDISSYIV (SEQ ID NO: 13); (e) a light chain variable region CDR2 comprising the amino acid sequence of HGTNLED (SEQ ID NO: 14); and (f) a light chain variable region CDR3 comprising the amino acid sequence of VHYAQFPYT (SEQ ID NO: 15). For example, the anti-OX40 antibody may comprise a heavy chain variable domain (VH) sequence comprising one, two or three CDRs selected from: (a) a heavy chain variable region CDR1 comprising the amino acid sequence of NYLIE (SEQ ID NO: 10); (b) a heavy chain variable region CDR2 comprising the amino acid sequence of VINPGSGDTYYSEKFKG (SEQ ID NO: 11); and (c) a heavy chain variable region CDR3 comprising the amino acid sequence of DRLDY (SEQ ID NO: 12) and/or a light chain variable domain (VL) sequence comprising one, two or three CDRs selected from (a) a light chain variable region CDR1 comprising the amino acid sequence of HASQDISSYIV (SEQ ID NO: 13); (b) a light chain variable region CDR2 comprising the amino acid sequence of HGTNLED (SEQ ID NO: 14); and (c) a light chain variable region CDR3 comprising the amino acid sequence of VHYAQFPYT (SEQ ID NO: 15). The anti-OX40 antibody may comprise the VH and VL sequences EVQLVQSGAE VKKPGASVKV SCKASGYAFT NYLIEWVRQA PGQGLEWIGV INPGSGDTYY SEKFKGRVTI TRDTSTSTAY LELSSLRSED TAVYYCARDR LDYWGQGTLV TVSS (SEQ ID NO: 16) and DIQMTQSPSS LSASVGDRVT ITCHASQDIS SYIVWYQQKP GKAPKLLIYH GTNLEDGVPS RFSGSGSGTD FTLTISSLQP EDFATYYCVH YAQFPYTFGQ GTKVEIK (SEQ ID NO: 17), respectively, including post-translational modifications of those sequences.

Further details regarding anti-OX40 antibodies are provided in WO 2015/153513, which is incorporated herein by reference in its entirety.

In certain examples, an antibody produced by the cells and methods disclosed herein is capable of binding to influenza virus B hemagglutinin, i.e., “fluB” (e.g., an antibody that binds hemagglutinin from the Yamagata lineage of influenza B viruses, binds hemagglutinin from the Victoria lineage of influenza B viruses, binds hemagglutinin from ancestral lineages of influenza B virus, or binds hemagglutinin from the Yamagata lineage, the Victoria lineage, and ancestral lineages of influenza B virus, in vitro and/or in vivo). Further details regarding anti-FluB antibodies are described in WO 2015/148806, which is incorporated herein by reference in its entirety.

In certain examples, an antibody produced by the cells and methods disclosed herein is capable of binding to low density lipoprotein receptor-related protein (LRP)-1 or LRP-8 or transferrin receptor, and at least one target selected from the group consisting of beta-secretase (BACE1 or BACE2), alpha-secretase, gamma-secretase, tau-secretase, amyloid precursor protein (APP), death receptor 6 (DR6), amyloid beta peptide, alpha-synuclein, Parkin, Huntingtin, p75 NTR, CD40 and caspase-6.

In certain examples, an antibody produced by the cells and methods disclosed herein is a human IgG2 antibody against CD40. In certain examples, the anti-CD40 antibody is RG7876.

In certain examples, the cells, cell lines and/or methods of the present disclosure can be used to produce a polypeptide. The polypeptide may be a targeted immunocytokine. The targeted immunocytokine may be a CEA-IL2v immunocytokine, for example the CEA-IL2v immunocytokine RG7813. The targeted immunocytokine may be a FAP-IL2v immunocytokine, for example the FAP-IL2v immunocytokine is RG7461.

In examples, the multispecific antibody (such as a bispecific antibody) produced by the cells, cell lines and/or or methods provided herein is capable of binding to CEA and at least one additional target molecule. The multispecific antibody (such as a bispecific antibody) produced according to methods provided herein may be capable of binding to a tumor targeted cytokine and at least one additional target molecule. The multispecific antibody (such as a bispecific antibody) produced according to methods provided herein may be fused to IL2v (i.e., an interleukin 2 variant) and bind an IL1-based immunocytokine and at least one additional target molecule. In examples, the multispecific antibody (such as a bispecific antibody) produced according to methods provided herein is a T-cell bispecific antibody (i.e., a bispecific T-cell engager or BiTE.

In certain examples, the multispecific antibody (such as a bispecific antibody) produced according to methods provided herein is capable of binding to at least two target molecules selected from: IL-1 alpha and IL-1 beta, IL-12 and IL-1S; IL-13 and IL-9; IL-13 and IL-4; IL-13 and IL-5; IL-5 and IL-4; IL-13 and IL-1beta; IL-13 and IL-25; IL-13 and TARC; IL-13 and MDC; IL-13 and MEF; IL-13 and TGF-˜; IL-13 and LHR agonist; IL-12 and TWEAK, IL-13 and CL25; IL-13 and SPRR2a; IL-13 and SPRR2b; IL-13 and ADAMS, IL-13 and PED2, IL17A and IL17F, CEA and CD3, CD3 and CD19, CD138 and CD20; CD138 and CD40; CD19 and CD20; CD20 and CD3; CD3S and CD13S; CD3S and CD20; CD3S and CD40; CD40 and CD20; CD-S and IL-6; CD20 and BR3, TNF alpha and TGF-beta, TNF alpha and IL-1 beta; TNF alpha and IL-2, TNF alpha and IL-3, TNF alpha and IL-4, TNF alpha and IL-5, TNF alpha and IL6, TNF alpha and IL8, TNF alpha and IL-9, TNF alpha and IL-10, TNF alpha and IL-11, TNF alpha and IL-12, TNF alpha and IL-13, TNF alpha and IL-14, TNF alpha and IL-15, TNF alpha and IL-16, TNF alpha and IL-17, TNF alpha and IL-18, TNF alpha and IL-19, TNF alpha and IL-20, TNF alpha and IL-23, TNF alpha and IFN alpha, TNF alpha and CD4, TNF alpha and VEGF, TNF alpha and MIF, TNF alpha and ICAM-1, TNF alpha and PGE4, TNF alpha and PEG2, TNF alpha and RANK ligand, TNF alpha and Te38, TNF alpha and BAFF,TNF alpha and CD22, TNF alpha and CTLA-4, TNF alpha and GP130, TNF a and IL-12p40, VEGF and Angiopoietin, VEGF and HER2, VEGF-A and HER2, VEGF-A and PDGF, HER1 and HER2, VEGFA and ANG2,VEGF-A and VEGF-C, VEGF-C and VEGF-D, HER2 and DR5,VEGF and IL-8, VEGF and MET, VEGFR and MET receptor, EGFR and MET, VEGFR and EGFR, HER2 and CD64, HER2 and CD3, HER2 and CD16, HER2 and HER3; EGFR (HER1) and HER2, EGFR and HER3, EGFR and HER4, IL-14 and IL-13, IL-13 and CD40L, IL4 and CD40L, TNFR1 and IL-1 R, TNFR1 and IL-6R and TNFR1 and IL-18R, EpCAM and CD3, MAPG and CD28, EGFR and CD64, CSPGs and RGM A; CTLA-4 and BTN02; IGF1 and IGF2; IGF1/2 and Erb2B; MAG and RGM A; NgR and RGM A; NogoA and RGM A; OMGp and RGM A; POL-1 and CTLA-4; and RGM A and RGM B.

In certain examples, the multispecific antibody (such as a bispecific antibody) produced according to methods provided herein is an anti-CEA/anti-CD3 bispecific antibody. The anti-CEA/anti-CD3 bispecific antibody is RG7802. In certain embodiments, the anti-CEA/anti-CD3 bispecific antibody comprises the amino acid sequences set forth in SEQ ID NOs: 18-21 are provided below:

(SEQ ID NO: 18)
DIQMTQSPSS LSASVGDRVT ITCKASAAVG TYVAWYQQKP
GKAPKLLIYS ASYRKRGVPS RFSGSGSGTD FTLTISSLQP
EDFATYYCHQ YYTYPLFTFG QGTKLEIKRT VAAPSVFIFP
PSDEQLKSGT ASVVCLLNNF YPREAKVQWK VDNALQSGNS
QESVTEQDSK DSTYSLSSTL TLSKADYEKH KVYACEVTHQ
GLSSPVTKSF NRGEC
(SEQ ID NO: 19)
QAVVTQEPSL TVSPGGTVTL TCGSSTGAVT TSNYANWVQE
KPGQAFRGLI GGTNKRAPGT PARFSGSLLG GKAALTLSGA
QPEDEAEYYC ALWYSNLWVF GGGTKLTVLS SASTKGPSVF
PLAPSSKSTS GGTAALGCLV KDYFPEPVTV SWNSGALTSG
VHTFPAVLQS SGLYSLSSVV TVPSSSLGTQ TYICNVNHKP
SNTKVDKKVE PKSC
(SEQ ID NO: 20)
QVQLVQSGAE VKKPGASVKV SCKASGYTFT EFGMNWVRQA
PGQGLEWMGW INTKTGEATY VEEFKGRVTF TTDTSTSTAY
MELRSLRSDD TAVYYCARWD FAYYVEAMDY WGQGTTVTVS
SASTKGPSVF PLAPSSKSTS GGTAALGCLV KDYFPEPVTV
SWNSGALTSG VHTFPAVLQS SGLYSLSSVV TVPSSSLGTQ
TYICNVNHKP SNTKVDKKVE PKSCDGGGGS GGGGSEVQLL
ESGGGLVQPG GSLRLSCAAS GFTFSTYAMN WVRQAPGKGL
EWVSRIRSKY NNYATYYADS VKGRFTISRD DSKNTLYLQM
NSLRAEDTAV YYCVRHGNFG NSYVSWFAYW GQGTLVTVSS
ASVAAPSVFI FPPSDEQLKS GTASVVCLLN NFYPREAKVQ
WKVDNALQSG NSQESVTEQD SKDSTYSLSS TLTLSKADYE
KHKVYACEVT HQGLSSPVTK SFNRGECDKT HTCPPCPAPE
AAGGPSVFLF PPKPKDTLMI SRTPEVTCVV VDVSHEDPEV
KFNWYVDGVE VHNAKTKPRE EQYNSTYRVV SVLTVLHQDW
LNGKEYKCKV SNKALGAPIE KTISKAKGQP REPQVYTLPP
CRDELTKNQV SLWCLVKGFY PSDIAVEWES NGQPENNYKT
TPPVLDSDGS FFLYSKLTVD KSRWQQGNVF SCSVMHEALH
NHYTQKSLSL SPGK
(SEQ ID NO: 21)
QVQLVQSGAE VKKPGASVKV SCKASGYTFT EFGMNWVRQA
PGQGLEWMG WINTKTGEATY VEEFKGRVTF TTDTSTSTAY
MELRSLRSDD TAVYYCARWD FAYYVEAMD YWGQGTTVTVS
SASTKGPSVF PLAPSSKSTS GGTAALGCLV KDYFPEPVTV
SWNSGALTS GVHTFPAVLQS SGLYSLSSVV TVPSSSLGTQ
TYICNVNHKP SNTKVDKKVE PKSCDKTHT CPPCPAPEAAG
GPSVFLFPPK PKDTLMISRT PEVTCVVVDV SHEDPEVKFN
WYVDGVEVH NAKTKPREEQY NSTYRVVSVL TVLHQDWLNG
KEYKCKVSNK ALGAPIEKTI SKAKGQPRE PQVCTLPPSRD
ELTKNQVSLS CAVKGFYPSD IAVEWESNGQ PENNYKTTPP
VLDSDGSFF LVSKLTVDKSR WQQGNVFSCS VMHEALHNHY
TQKSLSLSPG K

Further details regarding anti-CEA/anti-CD3 bispecific antibodies are provided in WO 2014/121712, which is incorporated herein by reference in its entirety.

In certain examples, a multispecific antibody (such as a bispecific antibody) produced by the cells and methods disclosed herein is an anti-VEGF/anti-angiopoietin bispecific antibody. In certain examples, the anti-VEGF/anti-angiopoietin bispecific antibody bispecific antibody is a Crossmab. In certain examples, the anti-VEGF/anti-angiopoietin bispecific antibody is RG7716. In certain examples, the anti-CEA/anti-CD3 bispecific antibody comprises the amino acid sequences set forth in SEQ ID NOs: 22-25 are provided below:

(SEQ ID NO: 22)
EVQLVESGGG LVQPGGSLRL SCAASGYDFT HYGMNWVRQA
PGKGLEWVGW INTYTGEPTY AADFKRRFTF SLDTSKSTAY
LQMNSLRAED TAVYYCAKYP YYYGTSHWYF DVWGQGTLVT
VSSASTKGPS VFPLAPSSKS TSGGTAALGC LVKDYFPEPV
TVSWNSGALT SGVHTFPAVL QSSGLYSLSS VVTVPSSSLG
TQTYICNVNH KPSNTKVDKK VEPKSCDKTH TCPPCPAPEA
AGGPSVFLFP PKPKDTLMAS RTPEVTCVVV DVSHEDPEVK
FNWYVDGVEV HNAKTKPREE QYNSTYRVVS VLTVLAQDWL
NGKEYKCKVS NKALGAPIEK TISKAKGQPR EPQVYTLPPC
RDELTKNQVS LWCLVKGFYP SDIAVEWESN GQPENNYKTT
PPVLDSDGSF FLYSKLTVDK SRWQQGNVFS CSVMHEALHN
AYTQKSLSLS PGK
(SEQ ID NO: 23)
QVQLVQSGAE VKKPGASVKV SCKASGYTFT GYYMHWVRQA
PGQGLEWMGW INPNSGGTNY AQKFQGRVTM TRDTSISTAY
MELSRLRSDD TAVYYCARSP NPYYYDSSGY YYPGAFDIWG
QGTMVTVSSA SVAAPSVFIF PPSDEQLKSG TASVVCLLNN
FYPREAKVQW KVDNALQSGN SQESVTEQDS KDSTYSLSST
LTLSKADYEK HKVYACEVTH QGLSSPVTKS FNRGECDKTH
TCPPCPAPEA AGGPSVFLFP PKPKDTLMAS RTPEVTCVVV
DVSHEDPEVK FNWYVDGVEV HNAKTKPREE QYNSTYRVVS
VLTVLAQDWL NGKEYKCKVS NKALGAPIEK TISKAKGQPR
EPQVCTLPPS RDELTKNQVS LSCAVKGFYP SDIAVEWESN
GQPENNYKTT PPVLDSDGSF FLVSKLTVDK SRWQQGNVFS
CSVMHEALHN AYTQKSLSLS PGK
(SEQ ID NO: 24)
DIQLTQSPSS LSASVGDRVT ITCSASQDIS NYLNWYQQKP
GKAPKVLIYF TSSLHSGVPS RFSGSGSGTD FTLTISSLQP
EDFATYYCQQ YSTVPWTFGQ GTKVEIKRTV AAPSVFIFPP
SDEQLKSGTA SVVCLLNNFY PREAKVQWKV DNALQSGNSQ
ESVTEQDSKD STYSLSSTLT LSKADYEKHK VYACEVTHQG
LSSPVTKSFN RGEC
(SEQ ID NO: 25)
SYVLTQPPSV SVAPGQTARI TCGGNNIGSK SVHWYQQKPG
QAPVLVVYDD SDRPSGIPER FSGSNSGNTA TLTISRVEAG
DEADYYCQVW DSSSDHWVFG GGTKLTVLSS ASTKGPSVFP
LAPSSKSTSG GTAALGCLVK DYFPEPVTVS WNSGALTSGV
HTFPAVLQSS GLYSLSSVVT VPSSSLGTQT YICNVNHKPS
NTKVDKKVEP KSC

In certain examples, the multispecific antibody (such as a bispecific antibody) produced by methods disclosed herein is an anti-Ang2/anti-VEGF bispecific antibody. The anti-Ang2/anti-VEGF bispecific antibody may be RG7221. The anti-Ang2/anti-VEGF bispecific antibody may have CAS Number 1448221-05-3.

Soluble antigens or fragments thereof, optionally conjugated to other molecules, may be used as immunogens for generating antibodies. For transmembrane molecules, such as receptors, fragments of these (e.g., the extracellular domain of a receptor) may be used as the immunogen. Alternatively, cells expressing the transmembrane molecule may be used as the immunogen. Such cells may be derived from a natural source (e.g., cancer cell lines) or may be cells which have been transformed by recombinant techniques to express the transmembrane molecule. Other antigens and forms thereof useful for preparing antibodies will be apparent to those in the art.

In certain examples, the polypeptide (e.g., antibodies) produced by the cells, cell lines and/or methods disclosed herein is capable of binding to can be further conjugated to a chemical molecule such as a dye or cytotoxic agent such as a chemotherapeutic agent, a drug, a growth inhibitory agent, a toxin (e.g., an enzymatically active toxin of bacterial, fungal, plant, or animal origin, or fragments thereof), or a radioactive isotope (i.e., a radioconjugate). An immunoconjugate comprising an antibody or bispecific antibody produced using the methods described herein may contain the cytotoxic agent conjugated to a constant region of only one of the heavy chains or only one of the light chains.

### Antibody Variants

Variants of the antibodies provided herein are contemplated. For example, it can be desirable to alter the binding affinity and/or other biological properties of the antibody. Amino acid sequence variants of an antibody can be prepared by introducing appropriate modifications into the nucleotide sequence encoding the antibody, or by peptide synthesis. Such modifications include, for example, deletions from, and/or insertions into and/or substitutions of residues within the amino acid sequences of the antibody. Any combination of deletion, insertion, and substitution can be made to arrive at the final construct, provided that the final construct possesses the desired characteristics, e.g., antigen-binding.

### Substitution, Insertion, and Deletion Variants

For example, antibody variants having one or more amino acid substitutions are provided. Sites of interest for substitutional mutagenesis include the CDRs and FRs. Examples of conservative substitutions are shown in Table 1 under the heading of “preferred substitutions”. Examples of more substantial changes are provided in Table 1 under the heading of “exemplary substitutions”, and as further described below in reference to amino acid side chain classes. Amino acid substitutions can be introduced into an antibody of interest and the products screened for a desired activity, e.g., retained/improved antigen binding, decreased immunogenicity, or improved ADCC or CDC.

TABLE 1
Amino acid substitutions

| Original | Exemplary | Preferred |
|---|---|---|
| Residue | Substitutions | Substitutions |
| Ala (A) | Val; Leu; Ile | Val |
| Arg (R) | Lys; Gln; Asn | Lys |
| Asn (N) | Gln; His; Asp, Lys; Arg | Gln |
| Asp (D) | Glu; Asn | Glu |
| Cys (C) | Ser; Ala | Ser |
| Gln (Q) | Asn; Glu | Asn |
| Glu (E) | Asp; Gln | Asp |
| Gly (G) | Ala | Ala |
| His (H) | Asn; Gln; Lys; Arg | Arg |
| Ile (I) | Leu; Val; Met; Ala; Phe; | Leu |
| | Norleucine | |
| Leu (L) | Norleucine; Ile; Val; Met; Ala; Phe | Ile |
| Lys (K) | Arg; Gln; Asn | Arg |
| Met (M) | Leu; Phe; Ile | Leu |
| Phe (F) | Trp; Leu; Val; Ile; Ala; Tyr | Tyr |
| Pro (P) | Ala | Ala |
| Ser (S) | Thr | Thr |
| Thr (T) | Val; Ser | Ser |
| Trp (W) | Tyr; Phe | Tyr |
| Tyr (Y) | Trp; Phe; Thr; Ser | Phe |
| Val (V) | Ile; Leu; Met; Phe; Ala; Norleucine | Leu |

Amino acids can be grouped according to common side-chain properties:
(1) hydrophobic: Norleucine, Met, Ala, Val, Leu, Ile;
(2) neutral hydrophilic: Cys, Ser, Thr, Asn, Gln;
(3) acidic: Asp, Glu;
(4) basic: His, Lys, Arg;
(5) residues that influence chain orientation: Gly, Pro; and
(6) aromatic: Trp, Tyr, Phe.
   Non-conservative substitutions generally entail exchanging a member of one of these classes for a member of another class.

One type of substitutional variant involves substituting one or more hypervariable region residues of a parent antibody (e.g., a humanized or human antibody). Generally, the resulting variant(s) selected for further study will have modifications (e.g., improvements) in certain biological properties (e.g., increased affinity, reduced immunogenicity) relative to the parent antibody and/or will have substantially retained certain biological properties of the parent antibody. An exemplary substitutional variant is an affinity matured antibody, which can be conveniently generated, e.g., using phage display-based affinity maturation techniques such as those described herein. Briefly, one or more. CDR residues are mutated and the variant antibodies displayed on phage and screened for a particular biological activity (e.g., binding affinity).

Alterations (e.g., substitutions) can be made in CDRs, e.g., to improve antibody affinity. Such alterations can be made in CDR “hotspots”, i.e., residues encoded by codons that undergo mutation at high frequency during the somatic maturation process (see, e.g., Chowdhury, Methods Mol. Biol. 207:179-196 (2008)), and/or residues that contact antigen, with the resulting variant VH or VL being tested for binding affinity. Affinity maturation by constructing and reselecting from secondary libraries has been described, e.g., in Hoogenboom et al. in Methods in Molecular Biology 178:1-37 (O'Brien et al., ed., Human Press, Totowa, NJ, (2001).) In some aspects of affinity maturation, diversity is introduced into the variable genes chosen for maturation by any of a variety of methods (e.g., error-prone PCR, chain shuffling, or oligonucleotide-directed mutagenesis). A secondary library is then created. The library is then screened to identify any antibody variants with the desired affinity. Another method to introduce diversity involves CDR-directed approaches, in which several CDR residues (e.g., 4-6 residues at a time) are randomized. CDR residues involved in antigen binding can be specifically identified, e.g., using alanine scanning mutagenesis or modeling. CDR-H3 and CDR-L3 in particular are often targeted.

Substitutions, insertions, or deletions may occur within one or more CDRs so long as such alterations do not substantially reduce the ability of the antibody to bind antigen. For example, conservative alterations (e.g., conservative substitutions as provided herein) that do not substantially reduce binding affinity can be made in the CDRs. Such alterations can, for example, be outside of antigen contacting residues in the CDRs. In certain variant VH and VL sequences provided above, each CDR either is unaltered, or contains no more than one, two or three amino acid substitutions.

A useful method for identification of residues or regions of an antibody that can be targeted for mutagenesis is called “alanine scanning mutagenesis” as described by Cunningham and Wells (1989) Science, 244:1081-1085. In this method, a residue or group of target residues (e.g., charged residues such as Arg, Asp, His, Lys, and Glu) are identified and replaced by a neutral or negatively charged amino acid (e.g., alanine or polyalanine) to determine whether the interaction of the antibody with antigen is affected. Further substitutions can be introduced at the amino acid locations demonstrating functional sensitivity to the initial substitutions. Alternatively, or additionally, a crystal structure of an antigen-antibody complex can be used to identify contact points between the antibody and antigen. Such contact residues and neighbouring residues can be targeted or eliminated as candidates for substitution. Variants can be screened to determine whether they contain the desired properties.

Amino acid sequence insertions include amino- and/or carboxyl-terminal fusions ranging in length from one residue to polypeptides containing a hundred or more residues, as well as intrasequence insertions of single or multiple amino acid residues. Examples of terminal insertions include an antibody with an N-terminal methionyl residue. Other insertional variants of the antibody molecule include the fusion to the N- or C-terminus of the antibody to an enzyme (e.g., for ADEPT (antibody directed enzyme prodrug therapy)) or a polypeptide which increases the serum half-life of the antibody.

### Glycosylation Variants

In certain examples, an antibody provided herein is altered to increase or decrease the extent to which the antibody is glycosylated. Addition or deletion of glycosylation sites to an antibody can be conveniently accomplished by altering the amino acid sequence such that one or more glycosylation sites is created or removed.

Where the antibody comprises an Fc region, the oligosaccharide attached thereto can be altered. Native antibodies produced by mammalian cells typically comprise a branched, biantennary oligosaccharide that is generally attached by an N-linkage to Asn297 of the CH2 domain of the Fc region. See, e.g., Wright et al. TIBTECH 15:26-32 (1997). The oligosaccharide can include various carbohydrates, e.g., mannose, N-acetyl glucosamine (GlcNAc), galactose, and sialic acid, as well as a fucose attached to a GlcNAc in the “stem” of the biantennary oligosaccharide structure. In some examples, modifications of the oligosaccharide in an antibody of the disclosure can be made in order to create antibody variants with certain improved properties.

In some examples, antibody variants are provided having a non-fucosylated oligosaccharide, i.e. an oligosaccharide structure that lacks fucose attached (directly or indirectly) to an Fc region. Such non-fucosylated oligosaccharide (also referred to as “afucosylated” oligosaccharide) particularly is an N-linked oligosaccharide which lacks a fucose residue attached to the first GlcNAc in the stem of the biantennary oligosaccharide structure. In examples, antibody variants are provided having an increased proportion of non-fucosylated oligosaccharides in the Fc region as compared to a native or parent antibody. For example, the proportion of non-fucosylated oligosaccharides can be at least about 20%, at least about 40%, at least about 60%, at least about 80%, or even about 100% (i.e., no fucosylated oligosaccharides are present). The percentage of non-fucosylated oligosaccharides is the (average) amount of oligosaccharides lacking fucose residues, relative to the sum of all oligosaccharides attached to Asn 297 (e. g. complex, hybrid and high mannose structures) as measured by MALDI-TOF mass spectrometry, as described in WO 2006/082515, for example. Asn297 refers to the asparagine residue located at about position 297 in the Fc region (EU numbering of Fc region residues); however, Asn297 can also be located about ±3 amino acids upstream or downstream of position 297, i.e., between positions 294 and 300, due to minor sequence variations in antibodies. Such antibodies having an increased proportion of non-fucosylated oligosaccharides in the Fc region can have improved FcγRIIIa receptor binding and/or improved effector function, in particular improved ADCC function. See, e.g., US 2003/0157108; US 2004/0093621.

Examples of cell lines capable of producing antibodies with reduced fucosylation include Lec13 CHO cells deficient in protein fucosylation (Ripka et al. Arch. Biochem. Biophys. 249:533-545 (1986); US 2003/0157108; and WO 2004/056312, especially at Example 11), and knockout cell lines, such as alpha-1,6-fucosyltransferase gene, FUT8, knockout CHO cells (see, e.g., Yamane-Ohnuki et al. Biotech. Bioeng. 87:614-622 (2004); Kanda, Y. et al., Biotechnol. Bioeng., 94(4):680-688 (2006); and WO 2003/085107), or cells with reduced or abolished activity of a GDP-fucose synthesis or transporter protein (see, e.g., US2004259150, US2005031613, US2004132140, US2004110282.

In further examples, antibody variants are provided with bisected oligosaccharides, e.g., in which a biantennary oligosaccharide attached to the Fc region of the antibody is bisected by GlcNAc. Such antibody variants can have reduced fucosylation and/or improved ADCC function as described above. Examples of such antibody variants are described, e.g., in Umana et al., Nat Biotechnol 17, 176-180 (1999); Ferrara et al., Biotechn Bioeng 93, 851-861 (2006); WO 99/54342; WO 2004/065540, WO 2003/011878.

Antibody variants with at least one galactose residue in the oligosaccharide attached to the Fc region are also provided. Such antibody variants can have improved CDC function. Such antibody variants are described, e.g., in WO 1997/30087; WO 1998/58964; and WO 1999/22764.

### Fc Region Variants

In certain examples, one or more amino acid modifications can be introduced into the Fc region of an antibody provided herein, thereby generating an Fc region variant. The Fc region variant can comprise a human Fc region sequence (e.g., a human IgG1, IgG2, IgG3 or IgG4 Fc region) comprising an amino acid modification (e.g., a substitution) at one or more amino acid positions.

The present disclosure contemplates an antibody variant that possesses some but not all effector functions, which make it a desirable candidate for applications in which the half life of the antibody in vivo is important yet certain effector functions (such as complement-dependent cytotoxicity (CDC) and antibody-dependent cell-mediated cytotoxicity (ADCC)) are unnecessary or deleterious. In vitro and/or in vivo cytotoxicity assays can be conducted to confirm the reduction/depletion of CDC and/or ADCC activities. For example, Fc receptor (FcR) binding assays can be conducted to ensure that the antibody lacks Fc□R binding (hence likely lacking ADCC activity), but retains FcRn binding ability. The primary cells for mediating ADCC, NK cells, express Fc□RIII only, whereas monocytes express Fc□RI, Fc□RII and Fc□RIII. FcR expression on hematopoietic cells is summarized in Table 3 on page 464 of Ravetch and Kinet, Annu. Rev. Immunol. 9:457-492 (1991). Non-limiting examples of in vitro assays to assess ADCC activity of a molecule of interest is described in U.S. Pat. No. 5,500,362 (see, e.g., Hellstrom, I. et al. Proc. Nat'l Acad. Sci. USA 83:7059-7063 (1986)) and Hellstrom, I et al., Proc. Nat'l Acad. Sci. USA 82:1499-1502 (1985); 5,821,337 (see Bruggemann, M. et al., J. Exp. Med. 166:1351-1361 (1987)). Alternatively, non-radioactive assays methods can be employed (see, for example, ACTI™ non-radioactive cytotoxicity assay for flow cytometry (CellTechnology, Inc. Mountain View, CA; and CytoTox 96® non-radioactive cytotoxicity assay (Promega, Madison, WI). Useful effector cells for such assays include peripheral blood mononuclear cells (PBMC) and Natural Killer (NK) cells. Alternatively, or additionally, ADCC activity of the molecule of interest can be assessed in vivo, e.g., in a animal model such as that disclosed in Clynes et al. Proc. Nat'l Acad. Sci. USA 95:652-656 (1998). C1q binding assays can also be carried out to confirm that the antibody is unable to bind C1q and hence lacks CDC activity. See, e.g., C1q and C3c binding ELISA in WO 2006/029879 and WO 2005/100402. To assess complement activation, a CDC assay can be performed (see, for example, Gazzano-Santoro et al., J. Immunol. Methods 202:163 (1996); Cragg, M. S. et al., Blood 101:1045-1052 (2003); and Cragg, M. S. and M. J. Glennie, Blood 103:2738-2743 (2004)). FcRn binding and in vivo clearance/half life determinations can also be performed using methods known in the art (see, e.g., Petkova, S. B. et al., Int'l. Immunol. 18(12):1759-1769 (2006); WO 2013/120929 A1).

Antibodies with reduced effector function include those with substitution of one or more of Fc region residues 238, 265, 269, 270, 297, 327 and 329 (U.S. Pat. No. 6,737,056). Such Fc mutants include Fc mutants with substitutions at two or more of amino acid positions 265, 269, 270, 297 and 327, including the so-called “DANA” Fc mutant with substitution of residues 265 and 297 to alanine (U.S. Pat. No. 7,332,581).

Certain antibody variants with improved or diminished binding to FcRs are described. (See, e.g., U.S. Pat. No. 6,737,056; WO 2004/056312, and Shields et al., J. Biol. Chem. 9(2): 6591-6604 (2001).)

In certain examples, an antibody variant comprises an Fc region with one or more amino acid substitutions which improve ADCC, e.g., substitutions at positions 298, 333, and/or 334 of the Fc region (EU numbering of residues).

In certain examples, an antibody variant comprises an Fc region with one or more amino acid substitutions which diminish FcγR binding, e.g., substitutions at positions 234 and 235 of the Fc region (EU numbering of residues). In one aspect, the substitutions are L234A and L235A (LALA). The antibody variant may further comprise D265A and/or P329G in an Fc region derived from a human IgG1 Fc region. The substitutions may be L234A, L235A and P329G (LALA-PG) in an Fc region derived from a human IgG1 Fc region. (See, e.g., WO 2012/130831). The substitutions may be L234A, L235A and D265A (LALA-DA) in an Fc region derived from a human IgG1 Fc region.

In some examples, alterations are made in the Fc region that result in altered (i.e., either improved or diminished) C1q binding and/or Complement Dependent Cytotoxicity (CDC), e.g., as described in U.S. Pat. No. 6,194,551, WO 99/51642, and Idusogie et al. J. Immunol. 164: 4178-4184 (2000).

Antibodies with increased half lives and improved binding to the neonatal Fc receptor (FcRn), which is responsible for the transfer of maternal IgGs to the fetus (Guyer et al., J. Immunol. 117:587 (1976) and Kim et al., J. Immunol. 24:249 (1994)), are described in US2005/0014934 (Hinton et al.). Those antibodies comprise an Fc region with one or more substitutions therein which improve binding of the Fc region to FcRn. Such Fc variants include those with substitutions at one or more of Fc region residues: 238, 252, 254, 256, 265, 272, 286, 303, 305, 307, 311, 312, 317, 340, 356, 360, 362, 376, 378, 380, 382, 413, 424 or 434, e.g., substitution of Fc region residue 434 (See, e.g., U.S. Pat. No. 7,371,826; Dall'Acqua, W. F., et al. J. Biol. Chem. 281 (2006) 23514-23524).

Fc region residues critical to the mouse Fc-mouse FcRn interaction have been identified by site-directed mutagenesis (see e.g. Dall'Acqua, W. F., et al. J. Immunol 169 (2002) 5171-5180). Residues I253, H310, H433, N434, and H435 (EU index numbering) are involved in the interaction (Medesan, C., et al., Eur. J. Immunol. 26 (1996) 2533; Firan, M., et al., Int. Immunol. 13 (2001) 993; Kim, J. K., et al., Eur. J. Immunol. 24 (1994) 542). Residues I253, H310, and H435 were found to be critical for the interaction of human Fc with murine FcRn (Kim, J. K., et al., Eur. J. Immunol. 29 (1999) 2819). Studies of the human Fc-human FcRn complex have shown that residues I253, S254, H435, and Y436 are crucial for the interaction (Firan, M., et al., Int. Immunol. 13 (2001) 993; Shields, R. L., et al., J. Biol. Chem. 276 (2001) 6591-6604). In Yeung, Y. A., et al. (J. Immunol. 182 (2009) 7667-7671) various mutants of residues 248 to 259 and 301 to 317 and 376 to 382 and 424 to 437 have been reported and examined.

In certain examples, an antibody variant comprises an Fc region with one or more amino acid substitutions, which reduce FcRn binding, e.g., substitutions at positions 253, and/or 310, and/or 435 of the Fc-region (EU numbering of residues). In certain examples, the antibody variant comprises an Fc region with the amino acid substitutions at positions 253, 310 and 435. The substitutions may be I253A, H310A and H435A in an Fc region derived from a human IgG1 Fc-region. See, e.g., Grevys, A., et al., J. Immunol. 194 (2015) 5497-5508.

In certain examples, an antibody variant comprises an Fc region with one or more amino acid substitutions, which reduce FcRn binding, e.g., substitutions at positions 310, and/or 433, and/or 436 of the Fc region (EU numbering of residues). In certain examples, the antibody variant comprises an Fc region with the amino acid substitutions at positions 310, 433 and 436. The substitutions may be H310A, H433A and Y436A in an Fc region derived from a human IgG1 Fc-region. (See, e.g., WO 2014/177460 A1).

In certain examples, an antibody variant comprises an Fc region with one or more amino acid substitutions which increase FcRn binding, e.g., substitutions at positions 252, and/or 254, and/or 256 of the Fc region (EU numbering of residues). In certain examples, the antibody variant comprises an Fc region with amino acid substitutions at positions 252, 254, and 256. In one aspect, the substitutions are M252Y, S254T and T256E in an Fc region derived from a human IgG1 Fc-region. See also Duncan & Winter, Nature 322:738-40 (1988); U.S. Pat. Nos. 5,648,260; 5,624,821; and WO 94/29351 concerning other examples of Fc region variants.

The C-terminus of the heavy chain of the antibody as reported herein can be a complete C-terminus ending with the amino acid residues PGK. The C-terminus of the heavy chain can be a shortened C-terminus in which one or two of the C terminal amino acid residues have been removed. In one preferred example, the C-terminus of the heavy chain is a shortened C-terminus ending PG. In one aspect of all aspects as reported herein, an antibody comprising a heavy chain including a C-terminal CH3 domain as specified herein, comprises the C-terminal glycine-lysine dipeptide (G446 and K447, EU index numbering of amino acid positions). In one aspect of all aspects as reported herein, an antibody comprising a heavy chain including a C-terminal CH3 domain, as specified herein, comprises a C-terminal glycine residue (G446, EU index numbering of amino acid positions).

### Cysteine Engineered Antibody Variants

In certain examples, it can be desirable to create cysteine engineered antibodies, e.g., THIOMAB™ antibodies, in which one or more residues of an antibody are substituted with cysteine residues. In particular examples, the substituted residues occur at accessible sites of the antibody. By substituting those residues with cysteine, reactive thiol groups are thereby positioned at accessible sites of the antibody and can be used to conjugate the antibody to other moieties, such as drug moieties or linker-drug moieties, to create an immunoconjugate, as described further herein. Cysteine engineered antibodies can be generated as described, e.g., in U.S. Pat. Nos. 7,521,541, 8,30,930, 7,855,275, 9,000,130, or WO 2016040856.

### Antibody Derivatives

In certain examples, an antibody provided herein can be further modified to contain additional nonproteinaceous moieties that are known in the art and readily available. The moieties suitable for derivatization of the antibody include but are not limited to water soluble polymers. Non-limiting examples of water soluble polymers include, but are not limited to, polyethylene glycol (PEG), copolymers of ethylene glycol/propylene glycol, carboxymethylcellulose, dextran, polyvinyl alcohol, polyvinyl pyrrolidone, poly-1, 3-dioxolane, poly-1,3,6-trioxane, ethylene/maleic anhydride copolymer, polyaminoacids (either homopolymers or random copolymers), and dextran or poly(n-vinyl pyrrolidone)polyethylene glycol, propylene glycol homopolymers, propylene oxide/ethylene oxide co-polymers, polyoxyethylated polyols (e.g., glycerol), polyvinyl alcohol, and mixtures thereof. Polyethylene glycol propionaldehyde can have advantages in manufacturing due to its stability in water. The polymer can be of any molecular weight, and can be branched or unbranched. The number of polymers attached to the antibody can vary, and if more than one polymer are attached, they can be the same or different molecules. In general, the number and/or type of polymers used for derivatization can be determined based on considerations including, but not limited to, the particular properties or functions of the antibody to be improved, whether the antibody derivative will be used in a therapy under defined conditions, etc.

### Immunoconjugates

The present disclosure also provides immunoconjugates comprising an antibody disclosed herein conjugated (chemically bonded) to one or more therapeutic agents such as cytotoxic agents, chemotherapeutic agents, drugs, growth inhibitory agents, toxins (e.g., protein toxins, enzymatically active toxins of bacterial, fungal, plant, or animal origin, or fragments thereof), or radioactive isotopes.

In some examples, an immunoconjugate is an antibody-drug conjugate (ADC) in which an antibody is conjugated to one or more of the therapeutic agents mentioned above. The antibody is typically connected to one or more of the therapeutic agents using linkers. An overview of ADC technology including examples of therapeutic agents and drugs and linkers is set forth in Pharmacol Review 68:3-19 (2016).

In other examples, an immunoconjugate comprises an antibody as described herein conjugated to an enzymatically active toxin or fragment thereof, including but not limited to diphtheria A chain, nonbinding active fragments of diphtheria toxin, exotoxin A chain (from Pseudomonas aeruginosa), ricin A chain, abrin A chain, modeccin A chain, alpha-sarcin, Aleurites fordii proteins, dianthin proteins, Phytolaca americana proteins (PAPI, PAPII, and PAP-S), Momordica charantia inhibitor, curcin, crotin, Sapaonaria officinalis inhibitor, gelonin, mitogellin, restrictocin, phenomycin, enomycin, and the tricothecenes.

In other examples, an immunoconjugate comprises an antibody as described herein conjugated to a radioactive atom to form a radioconjugate. A variety of radioactive isotopes are available for the production of radioconjugates. Examples include At211, I131, I125, Y90, Re186, Re188, Sm153, Bi212, P32, Pb212 and radioactive isotopes of Lu. When the radioconjugate is used for detection, it can comprise a radioactive atom for scintigraphic studies, for example tc99m or 1123, or a spin label for nuclear magnetic resonance (NMR) imaging (also known as magnetic resonance imaging, MRI), such as iodine-123 again, iodine-131, indium-111, fluorine-19, carbon-13, nitrogen-15, oxygen-17, gadolinium, manganese or iron.

Conjugates of an antibody and cytotoxic agent may be made using a variety of bifunctional protein coupling agents such as N-succinimidyl-3-(2-pyridyldithio) propionate (SPDP), succinimidyl-4-(N-maleimidomethyl) cyclohexane-1-carboxylate (SMCC), iminothiolane (IT), bifunctional derivatives of imidoesters (such as dimethyl adipimidate HCl), active esters (such as disuccinimidyl suberate), aldehydes (such as glutaraldehyde), bis-azido compounds (such as bis (p-azidobenzoyl) hexanediamine), bis-diazonium derivatives (such as bis-(p-diazoniumbenzoyl)-ethylenediamine), diisocyanates (such as toluene 2,6-diisocyanate), and bis-active fluorine compounds (such as 1,5-difluoro-2,4-dinitrobenzene). For example, a ricin immunotoxin can be prepared as described in Vitetta et al., Science 238:1098 (1987). Carbon-14-labeled 1-isothiocyanatobenzyl-3-methyldiethylene triaminepentaacetic acid (MX-DTPA) is an exemplary chelating agent for conjugation of radionucleotide to the antibody. See WO 94/11026. The linker may be a “cleavable linker” facilitating release of a cytotoxic drug in the cell. For example, an acid-labile linker, peptidase-sensitive linker, photolabile linker, dimethyl linker or disulfide-containing linker (Chari et al., Cancer Res. 52:127-131 (1992); U.S. Pat. No. 5,208,020) can be used.

The immunoconjugates or ADCs herein expressly contemplate, but are not limited to such conjugates prepared with cross-linker reagents including, but not limited to, BMPS, EMCS, GMBS, HBVS, LC-SMCC, MBS, MPBH, SBAP, SIA, SIAB, SMCC, SMPB, SMPH, sulfo-EMCS, sulfo-GMBS, sulfo-KMUS, sulfo-MBS, sulfo-SIAB, sulfo-SMCC, and sulfo-SMPB, and SVSB (succinimidyl-(4-vinylsulfone)benzoate) which are commercially available (e.g., from Pierce Biotechnology, Inc., Rockford, IL., U.S.A.).

### 5.7 Exemplary Non-Limiting Embodiments

A. In certain embodiments, the present disclosure is directed to an isolated eukaryotic cell line, wherein the cell line comprises a stable integrated loss-of-function or attenuation-of-function mutation in each of the Bax and Bak genes.
A1. In certain embodiments, the present disclosure is directed to the cell line of A, wherein the cell line comprises a stable integrated loss-of-function mutation in each of the Bax and Bak genes.
A2. In certain embodiments, the present disclosure is directed to the cell line of A or A1, wherein the cell line is an animal cell line or a fungal cell line.
A3. In certain embodiments, the present disclosure is directed to the cell line of A2, wherein the animal cell line is a mammalian cell line.
A4. In certain embodiments, the present disclosure is directed to the cell line of A3, wherein the mammalian cell line is a COS cell line, a VERO cell line, a HeLa cell line, a HEK 293 cell line, a PER-C6 cell line, a K562 cell line, a MOLT-4 cell line, a Ml cell line, a NS-1 cell line, a COS-7 cell line, a MDBK cell line, a MDCK cell line, a MRC-5 cell line, a WI-38 cell line, a WEHI cell line, a SP2/0 cell line, a BHK cell line or a CHO cell line, or their derivatives.
A5. In certain embodiments, the present disclosure is directed to the cell line of A4, wherein the CHO cell line is a CHO K1 cell line, a CHO K1SV cell line, a DG44 cell line, a DUKXB-11 cell line, a CHOK1S cell line, or a CHO K1M cell line, or their derivatives.
A6. In certain embodiments, the present disclosure is directed to the cell line of any of A-A5, wherein the cell line comprises a deletion in each of the Bax and Bak genes.
A7. In certain embodiments, the present disclosure is directed to the cell line of any of A-A6, wherein the cell line further comprises a viral genome and one or more polynucleotides encoding a viral capsid.
A8. In certain embodiments, the present disclosure is directed to the cell line of A-A7, wherein the cell line further comprises a polynucleotide encoding a product of interest.
A9. In certain embodiments, the present disclosure is directed to the cell line of A8, wherein the polynucleotide that encodes the product of interest is integrated in the cellular genome of the cell line at a targeted location.
A10. In certain embodiments, the present disclosure is directed to the cell line of A8, wherein the polynucleotide that encodes the product of interest is randomly integrated in the cellular genome of the cell line.
A11. In certain embodiments, the present disclosure is directed to the cell line of any of A8-A10, wherein the polynucleotide that encodes the product of interest is an extrachromosomal polynucleotide.
A12. In certain embodiments, the present disclosure is directed to the cell line of any of A8-A10, wherein the polynucleotide that encodes the product of interest is integrated into a chromosome of the cell line.
A13. In certain embodiments, the present disclosure is directed to the cell line of any of A8-A10, wherein product of interest comprises a recombinant polypeptide.
A14. In certain embodiments, the present disclosure is directed to the cell line of any of A8-A13, wherein the product of interest comprises an antibody, an antibody-fusion protein, an antigen, an enzyme, or a vaccine.
A15. In certain embodiments, the present disclosure is directed to the cell line of A14, wherein the antibody is a multispecific antibody or antigen-binding fragment thereof.
A16. In certain embodiments, the present disclosure is directed to the cell line of A14 or A15, wherein the antibody consists of a single heavy chain sequence and a single light chain sequence or antigen-binding fragments thereof.
A17. In certain embodiments, the present disclosure is directed to the cell line of any of A14-A16, wherein the antibody comprises a chimeric antibody, a human antibody or a humanized antibody.
A18. In certain embodiments, the present disclosure is directed to the cell line of any of A14-A17, wherein the antibody comprises a monoclonal antibody.
A19. In certain embodiments, the present disclosure is directed to the cell line of any of A6-A18, wherein the cell line has a higher specific productivity than a corresponding isolated eukaryotic cell line that comprises the polynucleotide and functional copies of each of the wild type Bax and Bak genes.
A20. In certain embodiments, the present disclosure is directed to the cell line of any of A-A19, wherein the cell line is more resistant to apoptosis than a corresponding isolated eukaryotic cell line that comprises functional copies of each of the Bax and Bak genes.
A21. In certain embodiments, the present disclosure is directed to the cell line of any of A-A20, wherein the cell line is employed in cell culture processes such as fed-batch, perfusion, process intensified, semi-continuous perfusion, or continuous perfusion.
A22. In certain embodiments, the present disclosure is directed to the cell line of A21, wherein the cell line is employed in an intensified perfusion process.
A23. In certain embodiments, the present disclosure is directed to a composition comprising a eukaryotic cell line according to any of A-A22.
A24. In certain embodiments, the present disclosure is directed to the composition of A23, further comprising a cell culture medium.
B. In certain embodiments, the present disclosure is directed to a cell culture comprising a cell culture medium and a plurality of eukaryotic cells, wherein each cell of the plurality comprises a stable integrated loss-of-function or attenuation-of-function mutation in each of the Bax and Bak genes.
B1. In certain embodiments, the present disclosure is directed to the cell culture of B, wherein each cell comprises a stable integrated loss-of-function mutation in each of the Bax and Bak genes.
B2. In certain embodiments, the present disclosure is directed to the cell culture of B or B1, wherein each cell of the plurality comprises a deletion in each of the Bax and Bak genes.
B3. In certain embodiments, the present disclosure is directed to the cell culture of any of B-B2, wherein the cells are animal cells or fungal cells.
B4. In certain embodiments, the present disclosure is directed to the cell culture of B3, wherein the animal cells are mammalian cells.
B5. In certain embodiments, the present disclosure is directed to the cell culture of B4, wherein the mammalian cells are COS cells, VERO cells, HeLa cells, HEK 293 cells, PER-C6 cells, K562 cells, MOLT-4 cells, Ml cells, NS-1 cells, COS-7 cells, MDBK cells, MDCK cells, MRC-5 cells, WI-38 cells, WEHI cells, SP2/0 cells, BHK cells or a CHO cells, or their derivatives.
B6. In certain embodiments, the present disclosure is directed to the cell culture of B5, wherein the CHO cells are a CHO K1 cells, CHO K1SV cells, DG44 cells, DUKXB-11 cells, CHOK1S cells, or CHO K1M cells, or their derivatives.
B7. In certain embodiments, the present disclosure is directed to the cell culture of any of B-B6, wherein the cell culture further comprises a polynucleotide that encodes a product of interest.
B8. In certain embodiments, the present disclosure is directed to the cell culture of B7, wherein the polynucleotide that encodes the product of interest is integrated in the cellular genome of the cells at a targeted location.
B9. In certain embodiments, the present disclosure is directed to the cell culture of B7, wherein the polynucleotide that encodes the product of interest is randomly integrated in the cellular genome of the cells.
B10. In certain embodiments, the present disclosure is directed to the cell culture of any of B7-B9, wherein the polynucleotide that encodes the product of interest is an extrachromosomal polynucleotide.
B11. In certain embodiments, the present disclosure is directed to the cell culture of any of B7-B9, wherein the polynucleotide that encodes the product of interest is integrated into a chromosome of the cells.
B12. In certain embodiments, the present disclosure is directed to the cell culture of any of B7-B11, wherein the product of interest comprises a recombinant polypeptide.
B13. In certain embodiments, the present disclosure is directed to the cell culture of any of B7-B12, wherein the product of interest is an antibody, an antibody-fusion protein, an antigen, an enzyme, or a vaccine.
B14. In certain embodiments, the present disclosure is directed to the cell culture of B13, wherein the antibody is a multispecific antibody or antigen-binding fragment thereof.
B15. In certain embodiments, the present disclosure is directed to the cell culture of B13 or B14, wherein the antibody consists of a single heavy chain sequence and a single light chain sequence or antigen-binding fragments thereof.
B16. In certain embodiments, the present disclosure is directed to the cell culture of any of B13-B15, wherein the antibody comprises a chimeric antibody, a human antibody or a humanized antibody.
B17. In certain embodiments, the present disclosure is directed to the cell culture of any of B13-B16, wherein the antibody comprises a monoclonal antibody.
B18. In certain embodiments, the present disclosure is directed to the cell culture of any of B-B17, wherein the each of the cells further comprise a recombinant polynucleotide.
B19. In certain embodiments, the present disclosure is directed to the cell culture of B-B18, wherein the cells are employed in a cell culture process such as fed-batch, perfusion, process intensified, semi-continuous perfusion, or continuous perfusion.
B20. In certain embodiments, the present disclosure is directed to the cell culture of B19, wherein the cells are employed in an intensified perfusion process.
C. In certain embodiments, the present disclosure is directed to a method of reducing apoptotic activity in a eukaryotic cell, comprising administering to the cell a genetic engineering system, wherein the genetic engineering system: a) knocks down or knocks out the expression of a Bax polypeptide isoform; and b) knocks down or knocks out the expression of a Bak polypeptide isoform.
C1. In certain embodiments, the present disclosure is directed to the method of C, wherein the method further comprises employing the eukaryotic cell in a fed-batch, perfusion, process intensified, semi-continuous perfusion, or continuous perfusion cell culture process.
C2. In certain embodiments, the present disclosure is directed to the method of C1, wherein the eukaryotic cell is employed in an intensified cell culture process.
C3. In certain embodiments, the present disclosure is directed to the method of any of C-C2, wherein the genetic engineering system is selected from the group consisting of a CRISPR/Cas system, a zinc-finger nuclease (ZFN) system, a transcription activator-like effector nuclease (TALEN) system and a combination thereof.
C4. In certain embodiments, the present disclosure is directed to the method of any C-C3, wherein the genetic engineering system is or comprises a CRISPR/Cas9 system.
C5. In certain embodiments, the present disclosure is directed to the method of C4, wherein the CRISPR/Cas9 system comprises: a) a Cas9 molecule, b) at least one first guide RNA (gRNA) comprising a targeting sequence that is complementary to a target sequence in a Bax gene, and c) at least one second gRNA comprising a targeting sequence that is complementary to a target sequence in a Bak gene.
C6. In certain embodiments, the present disclosure is directed to the method of C5, wherein at least one of the target sequences is a portion of the Bax gene, and/or wherein at least one of the target sequences is a portion of the Bak gene.
C7. In certain embodiments, the present disclosure is directed to the method of any of C-C6, wherein the expression of the Bax polypeptide and/or the expression of the Bak polypeptide is knocked out, and the apoptotic activity of the cell is reduced compared to the apoptotic activity of a reference cell.
C8. In certain embodiments, the present disclosure is directed to the method of any of C-C6, wherein the expression of the Bax polypeptide and/or the expression of the Bak polypeptide is knocked down, and the apoptotic activity of the cell is reduced compared to the apoptotic activity of a reference cell.
C9. In certain embodiments, the present disclosure is directed to the method of C7 or C8, wherein the apoptotic activity of the cell is determined from the viability for a population of said cells compared to the viability of a population of said reference cells determined at day 14 of a production phase.
C10. In certain embodiments, the present disclosure is directed to the method of any of C7-C9, wherein the reference cell is a cell that comprises wild-type alleles of the Bax and Bak genes.
C11. In certain embodiments, the present disclosure is directed to the method of any of C-C10, wherein the genetic engineering system is or comprises a zinc-finger nuclease (ZFN) system or a transcription activator-like effector nuclease (TALEN) system.
C12. In certain embodiments, the present disclosure is directed to the method of any of C-C11, wherein the cell line development system comprises targeted integration, random integration or transposase systems.
C13. In certain embodiments, the present disclosure is directed to the method of any of C-C12, wherein the cell is an animal cell or a fungal cell.
C14. In certain embodiments, the present disclosure is directed to the method of C13, wherein the animal cell is a mammalian cell.
C15. In certain embodiments, the present disclosure is directed to the method of C14, wherein the mammalian cell is a COS cell, a VERO cell, a HeLa cell, a HEK 293 cell, a PER-C6 cell, a K562 cell, a MOLT-4 cell, a Ml cell, NS-1 cell, a COS-7 cell, a MDBK cell, a MDCK cell, a MRC-5 cell, a WI-38 cell, a WEHI cell, a SP2/0 cell line, a BHK cell or a CHO cell line, or their derivatives.
C16. In certain embodiments, the present disclosure is directed to the method of C15, wherein the CHO cell is a CHO K1 cell, a CHO K1SV cell, a DG44 cell, a DUKXB-11 cell, a CHOK1S cell, or a CHO K1M cell, or their derivatives.
C17. In certain embodiments, the present disclosure is directed to the method of any of C-C16, wherein the cell further comprises a polynucleotide that encodes a product of interest.
C18. In certain embodiments, the present disclosure is directed to the method of C17, wherein the polynucleotide that encodes the product of interest is integrated in the cellular genome of the cell at a targeted location.
C19. In certain embodiments, the present disclosure is directed to the method of C17, wherein the polynucleotide that encodes the product of interest is randomly integrated in the cellular genome of the cell.
C20. In certain embodiments, the present disclosure is directed to the method of any of C17-C19, wherein the polynucleotide that encodes the product of interest is an extrachromosomal polynucleotide.
C21. In certain embodiments, the present disclosure is directed to the method of any of C17-C19, wherein the polynucleotide that encodes the product of interest is integrated into a chromosome of the cell.
C22. In certain embodiments, the present disclosure is directed to the method of any of C17-C21, wherein the product of interest comprises a recombinant polypeptide.
C23. In certain embodiments, the present disclosure is directed to the method of any of C17-C22, wherein the product of interest is an antibody, an antibody-fusion protein, an antigen, an enzyme, or a vaccine.
C24. In certain embodiments, the present disclosure is directed to the method of C23, wherein the antibody is a multispecific antibody or antigen-binding fragment thereof.
C25. In certain embodiments, the present disclosure is directed to the method of C23 or C24, wherein the antibody consists of a single heavy chain sequence and a single light chain sequence or antigen-binding fragments thereof.
C26. In certain embodiments, the present disclosure is directed to the method of any of C23-C25, wherein the antibody comprises a chimeric antibody, a human antibody or a humanized antibody.
C27. In certain embodiments, the present disclosure is directed to the method of any of C23-C26, wherein the antibody comprises a monoclonal antibody.
C28. In certain embodiments, the present disclosure is directed to the method of any of C23-C27, wherein the each of the cells further comprise a recombinant polynucleotide.
D. In certain embodiments, the present disclosure is directed to a method of producing a recombinant polypeptide, comprising: culturing a eukaryotic cell line that comprises: (a) a stable integrated loss-of-function or attenuation-of-function mutation in each of the Bax and Bak genes, and (b) a polynucleotide encoding the recombinant polypeptide, under conditions suitable for production of the polypeptide.
D1. In certain embodiments, the present disclosure is directed to the method of D, wherein the polynucleotide that encodes the polypeptide is integrated in the cellular genome of the cells of the cell line at a targeted location.
D2. In certain embodiments, the present disclosure is directed to the method of D, wherein the polynucleotide that encodes the polypeptide is randomly integrated in the cellular genome of the cells of the cell line.
D3. In certain embodiments, the present disclosure is directed to the method of any of D-D2, wherein the polynucleotide that encodes the polypeptide is an extrachromosomal polynucleotide.
D4. In certain embodiments, the present disclosure is directed to the method of any of D-D3, wherein the polynucleotide that encodes the polypeptide is integrated into a chromosome of the cells of the cell line.
D5. In certain embodiments, the present disclosure is directed to the method of any of D-D4, wherein the recombinant polypeptide is an antibody, an antibody-fusion protein, an antigen, an enzyme, or a vaccine.
D6. In certain embodiments, the present disclosure is directed to the method of D5, wherein the antibody is a multispecific antibody or antigen-binding fragment thereof.
D7. In certain embodiments, the present disclosure is directed to the method of D5 or D6, wherein the antibody consists of a single heavy chain sequence and a single light chain sequence or antigen-binding fragments thereof.
D8. In certain embodiments, the present disclosure is directed to the method of any of D5-D7, wherein the antibody comprises a chimeric antibody, a human antibody or a humanized antibody.
D9. In certain embodiments, the present disclosure is directed to the method of any of D5-D8, wherein the antibody comprises a monoclonal antibody.
D10. In certain embodiments, the present disclosure is directed to the method of any of D-D9, further comprising isolating the recombinant polypeptide.
E. In certain embodiments, the present disclosure is directed to a method of producing a viral vector, comprising: culturing a eukaryotic cell line that comprises (a) stable integrated a loss-of-function or attenuation-of function mutation in each of the Bax and Bak genes, (b) a viral genome, and (c) one or more polynucleotides encoding a viral capsid, under conditions suitable for production of the viral vector.
E1. In certain embodiments, the present disclosure is directed to the method of E, further comprising isolating the viral vector.
E2. In certain embodiments, the present disclosure is directed to the method of any of E-E1, wherein the cell line is an animal cell line, or a fungal cell line.
E3. In certain embodiments, the present disclosure is directed to the method of E2, wherein the animal cell line is a mammalian cell line.
E4. In certain embodiments, the present disclosure is directed to the method of E3, wherein the mammalian cell line is a COS cell line, a VERO cell line, a HeLa cell line, a HEK 293 cell line, a PER-C6 cell line, a K562 cell line, a MOLT-4 cell line, a Ml cell line, a NS-1 cell line, a COS-7 cell line, a MDBK cell line, a MDCK cell line, a MRC-5 cell line, a WI-38 cell line, a WEHI cell line, a SP2/0 cell line, a BHK cell line or a CHO cell line, or their derivatives.
E5. In certain embodiments, the present disclosure is directed to the method of E4, wherein the CHO cell line is a CHO K1 cell line, a CHO K1SV cell line, a DG44 cell line, a DUKXB-11 cell line, a CHOK1S cell line, or a CHO K1M cell line, or their derivatives.
E6. In certain embodiments, the present disclosure is directed to the method of any of E-E5, wherein the cell line is cultured in a cell culture medium.
E7. In certain embodiments, the present disclosure is directed to the method of any of E-E6, wherein the cell line is cultured under fed-batch culture conditions, or perfusion culture conditions.
E8. In certain embodiments, the present disclosure is directed to the method of E7, wherein the cell line is cultured under fed-batch culture conditions, optionally wherein the fed-batch culture conditions are intensified fed-batch culture conditions.
E9. In certain embodiments, the present disclosure is directed to the method of any of E-E8, wherein the cell line is cultured under perfusion culture conditions, optionally wherein the perfusion culture conditions are semi-continuous perfusion or continuous perfusion.
E10. In certain embodiments, the present disclosure is directed to the method of any E-E9, wherein the cell line comprises a stable integrated loss-of-function mutation in each of the Bax and Bak genes.

### 6. EXAMPLES

The disclosure will be more fully understood by reference to the following examples. They should not, however, be construed as limiting the scope of the disclosure. It is understood that the examples and embodiments described herein are for illustrative purposes only and that various modifications or changes in light thereof will be suggested to persons skilled in the art and are to be included within the spirit and purview of this application and scope of the appended claims.

### Example 1: Generation and Testing of Apoptosis Resistant Cells

This example describes an evaluation of cell lines generated from apoptotic resistant host in a 14-day intensified process. In this experiment, a standard molecule (antibody A) was tested as a model molecule. Generation of stable cell lines expressing antibody A followed a Standard Cell Line Development (CLD) Protocol. Top clones generated from wild-type (WT) targeted integration (TI) CHO host or two engineered hosts with both Bax and Bak genes knocked-out (Bax/Bak DKO) were evaluated in a CHO production media in an ambr15 minibioreactor using a 14-day intensified process. In the first 7 days of the process, the WT clones and Bax/Bak DKO clones showed similar titer, cell growth, viability and Qp. However, between Day 7 and Day 14, WT clones showed declined viability and Qp, while Bax/Bak DKO clones viability remained high. The top Bax/Bak DKO clone showed 50% titer increase compared to the top WT clone in this 14-day extended intensitied process. This indicates that blocking apoptosis by knocking out Bax and Bak genes improves production in intensified process. Additionally, this should enable extension of the production phase in an intensified process, achieving higher titers. This reduces the cost of manufacturing process as well as the number of manufacturing runs required to obtain a given amount of desired product.

### Methods

### Bax/Bak DKO Host Generation

Wild type TI host cells were co-transfected with Cas9 protein and gRNAs that target Bax and Bak genes. Transfected cells were single-cell printed at 1 cell/well in imaging quality 384-well plates (Corning #7311) prefilled with 40 μL proprietary seed train media containing selection reagents using the Cytena's single-cell printer and immediately imaged in white light and fluorescence modes using the Celigo Imager. The plates were incubated at 37° C., 5% CO₂, in a humidified environment for 2 weeks before 48 colonally-derived clones were picked based on confluence. Clones were expanded in the host seed train media and evaluated for Bax/Bak DKO efficiency by western blot analysis. Clones confirmed for Bax/Bak DKO were scaled up to generate Antibody A expressing clones.

### Single Cell Cloning of Antibody a Expressing Cells

Wild-type TI host cell line and Bax/Bak DKO host cell lines were used to generate Antibody A expressing targeted integration single cell clones. Transfection and single cell cloning was performed according to a standard cell line development protocol.

Cells were single-cell printed at 1 cell/well in 384-well plates prefilled with 40 μL single cell cloning (SCC) media containing selection reagents, 88 clones from each host were picked and transferred to 96-well plates. After three rounds of HTRF titer screening assays, 5 clonally-derived single cell clones per host were selected for fed-batch production assay evaluation in the ambr15.

### Clone Evaluation

Clone evaluation was performed in ambr15 intensified process with CHO production media (proprietary) for 14 days. All clones were scaled up in shake flasks for the N-1 passage. After 4 days of culture, cells were concentrated by centrifugation and inoculated at a high seeding density on day 0 of production

The culture temperature was maintained at 35° C. through the duration of the production evaluation. Appropriate feeds at 15% (of the working volume), and at 2.6% (of working volume) was added on days 1, 3, 5, 12 and on day 7 or 9 (if osmolarity is low). Clones were harvested on day 14. Table 2 provides an overview of the assay types and their respective sample collection days.

TABLE 2
Sampling and assays

| Sample Type | Sample Collection Days |
|---|---|
| NOVA FLEX2 (viability, viable | All days |
| cell count, lactate, glucose, pH) | |
| (titer) | Day 3, 7, 10, 14 |
| (charge variant) | Day 14 |
| aggregate) | Day 14 |
| (HILIC glycan assay) | Day 14 |
| amino acids concentration) | Day 3, 7, 10, 14 |

### Results

### Viability and Cell Growth

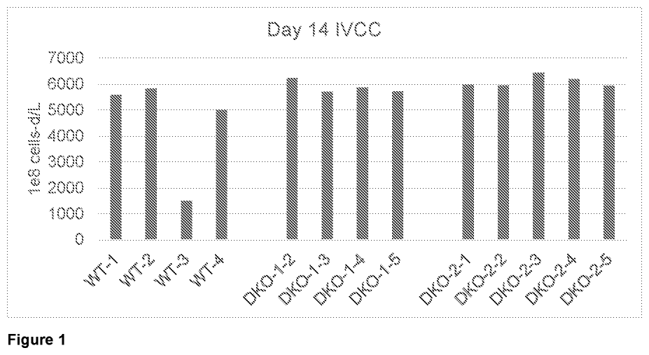

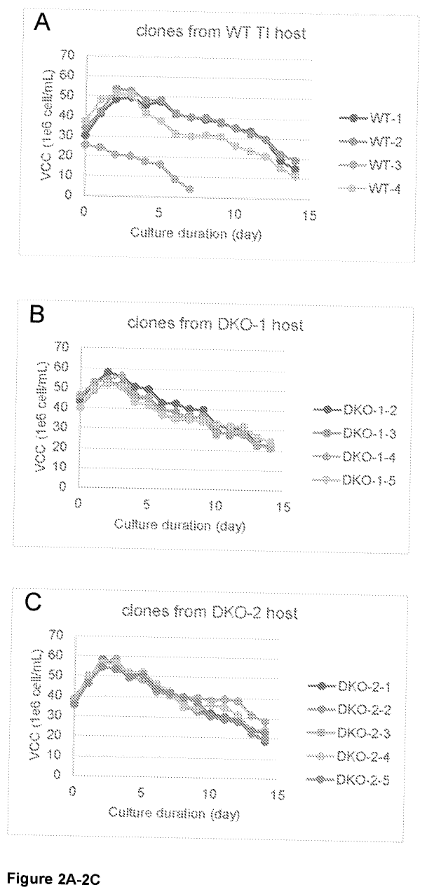

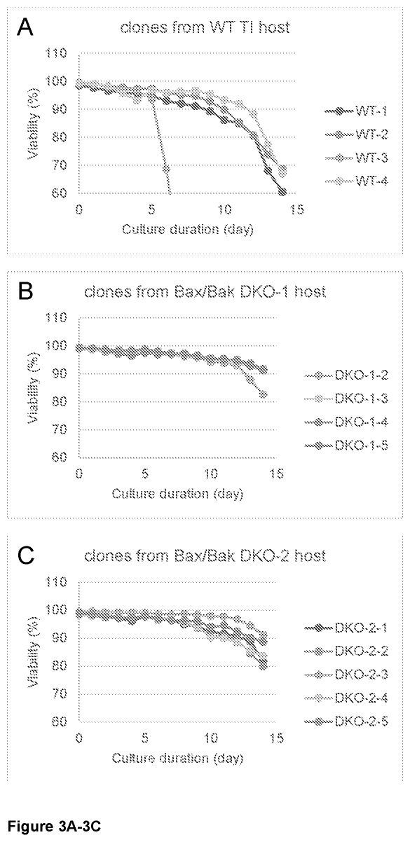

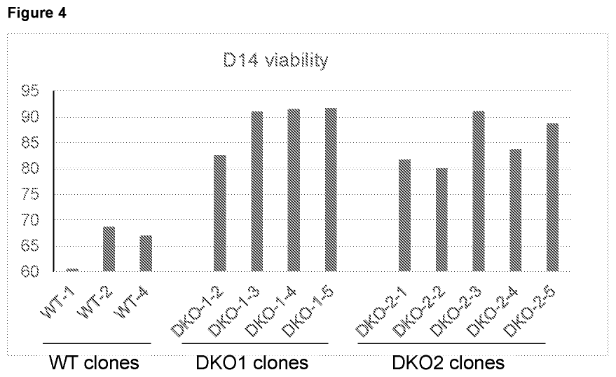

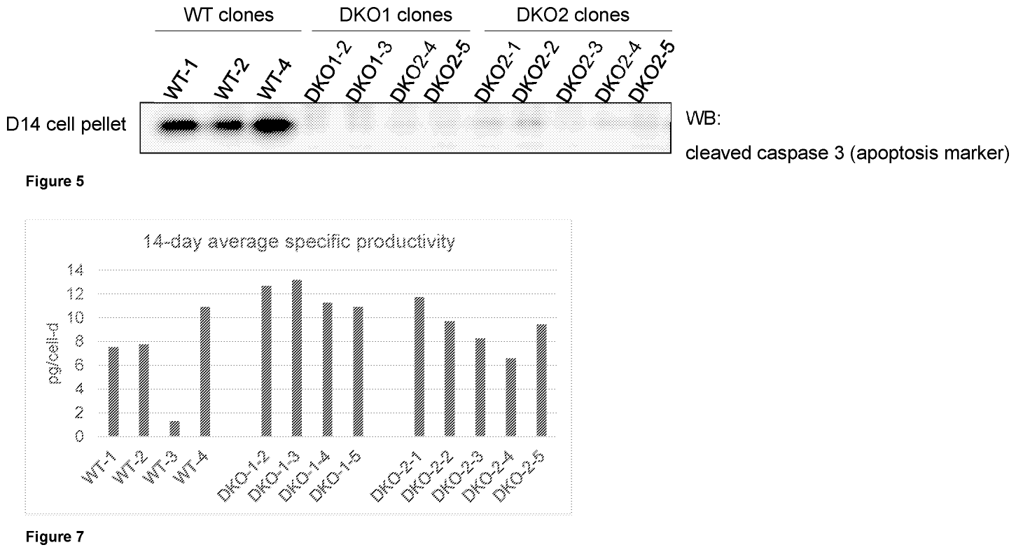

All the Bax/Bak DKO clones had viable cell count (VCC) comparable to or higher than WT clones throughout the 14-day process (FIGS. 1 and 2). VCC is determined per unit of volume, so dilution due to addition of feed resulted in some reduction in VCC in all clones over the production period. This is corrected for when calculating the viability (%). WT clones showed declined viability after Day 10 while Bax/Bak DKO clones viabilities remained high till the end of the process (FIG. 3). On Day 14, all WT clones had lower than 70% viability, while the Bax/Bak DKO clones had over 80% viability (FIG. 4). Western blot analysis of cleaved caspase 3 indicated that all WT clones were undergoing apoptosis at the end of the run while the Bax/Bak DKO clones were not (FIG. 5).

### Titer and Specific Productivity

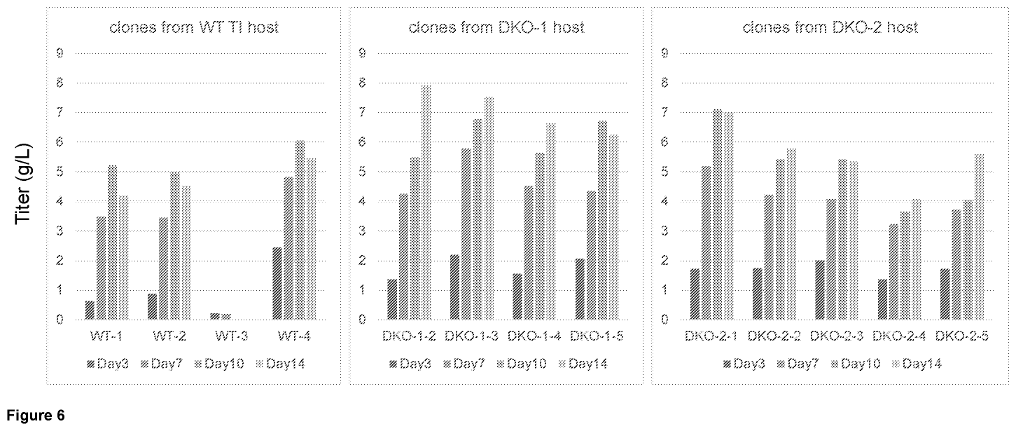

Day 3, 7, 10, and 14 titers and day 14 specific productivities are shown in FIGS. 6 and 7 respectively. Day 7 titers of WT and Bax/Bak DKO clones were comparable. However, on day 14, top clones generated from Bax/Bak DKO hosts showed higher titers than WT clones. More importantly, productivity of WT clones declined significantly after day 10, while Bax/Bak DKO clones still produced antibody. Note that the feeding strategy in this experiment was not optimized, several Bax/Bak DKO clones ran out of essential amino acids on day 7 and day 10. With further optimization of the feeding strategy, the titers of these Bax/Bak DKO clones would be expected to be higher.

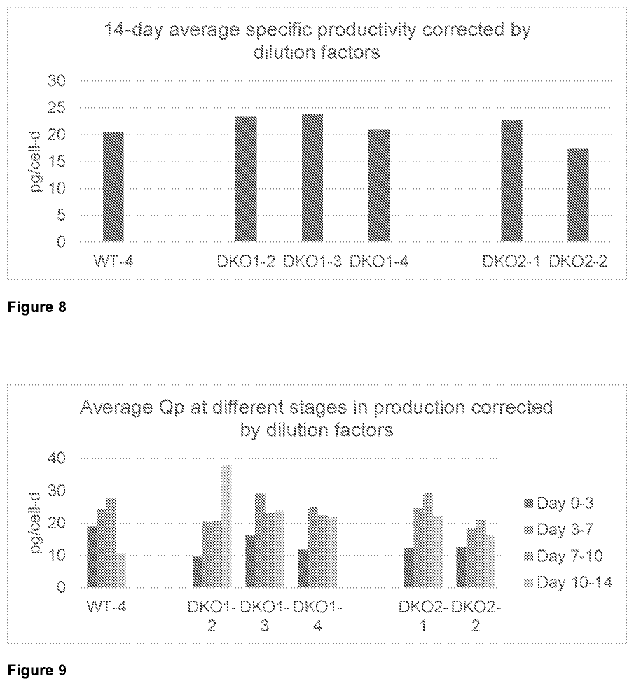

As cell culture was diluted every day during the process, by removing cell culture for sampling or volume reduction and adding feeds, specific productivities shown in FIG. 7 would represent an underestimate. In order to calculate the specific productivities at different stages during the 14-day process, the dilution factors were used to correct titer and VCC readings. The top clone generated from WT TI host, WT-4, together with top clones generated from the two Bax/Bak DKO hosts were analyzed for specific productivities in the whole 14-day process (FIG. 8) and at different stages during the process (FIG. 9). As FIG. 9 illustrates, the specific productivity of WT-4 clone significantly decreased between Day 10 and 14, while the specific productivities of all Bax/Bak DKO clones remained high after Day 10.

### Metabolites

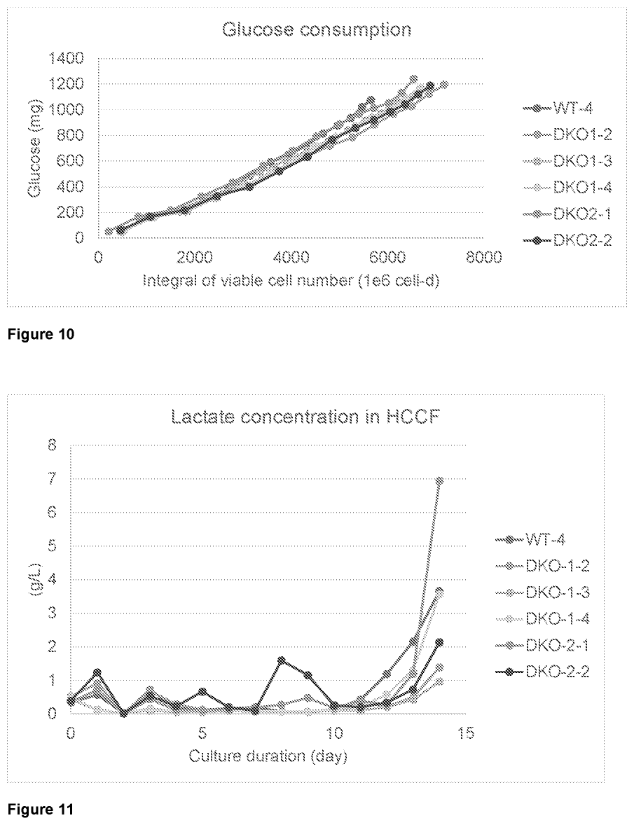

The glucose feeding strategy in the beginning was performed if the glucose readings were below or expected to be below optimal levels in the culture, further glucose was added. On Day 2, all of the clones ran out of glucose, but they were only fed with standard amounts of glucose. From Day 4 till the end, daily culture glucose consumption amounts were calculated, and additional glucose was added to make sure glucose reading will not be below the desired threshold of 2 g/L the next day. FIG. 10 provides a glucose consumption summary of the top clones. FIG. 11 provides a lactate summary of the top clones.

### Product Quality

Product quality data from day 14 PQA analysis suggest that product generated from Bax/Bak DKO clones had comparable product qualities to the WT clones.

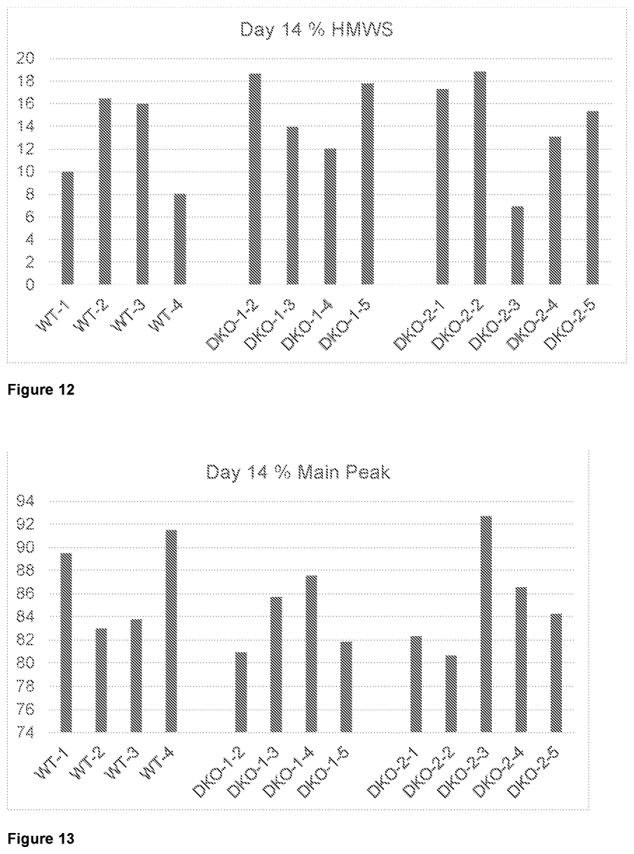

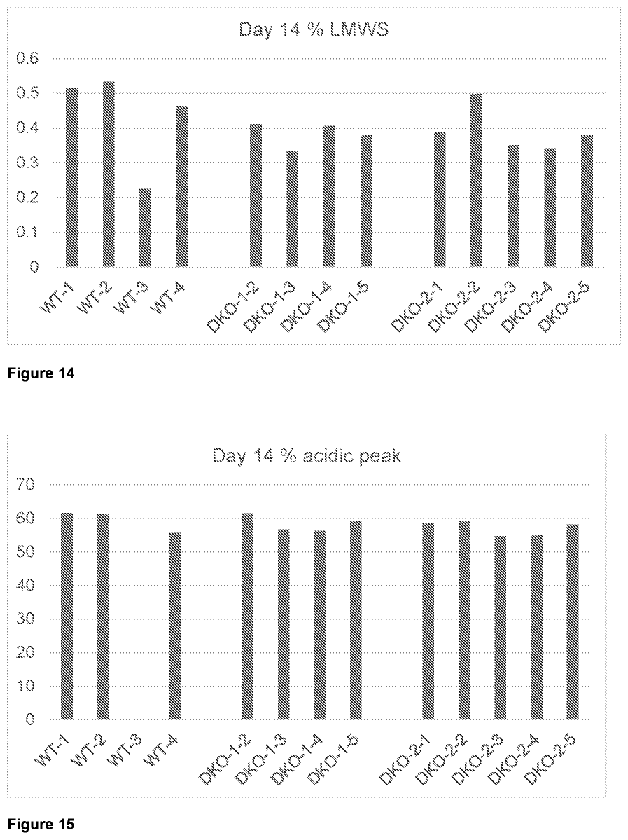

Size Variants (%): HMWS, Main Peak, LMWS. FIGS. 12, 13, and 14 show molecular size data for WT and Bax/Bak DKO clones. The aggregation data is comparable between WT and Bax/Bak DKO clones.

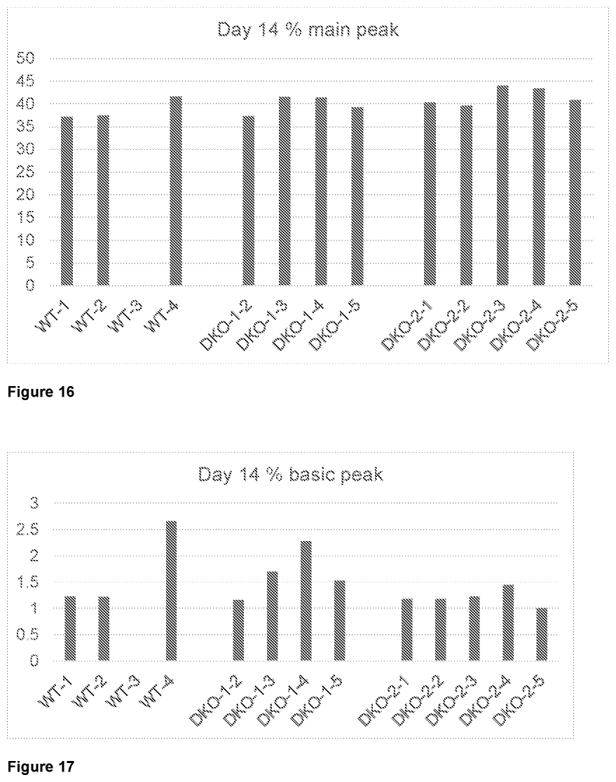

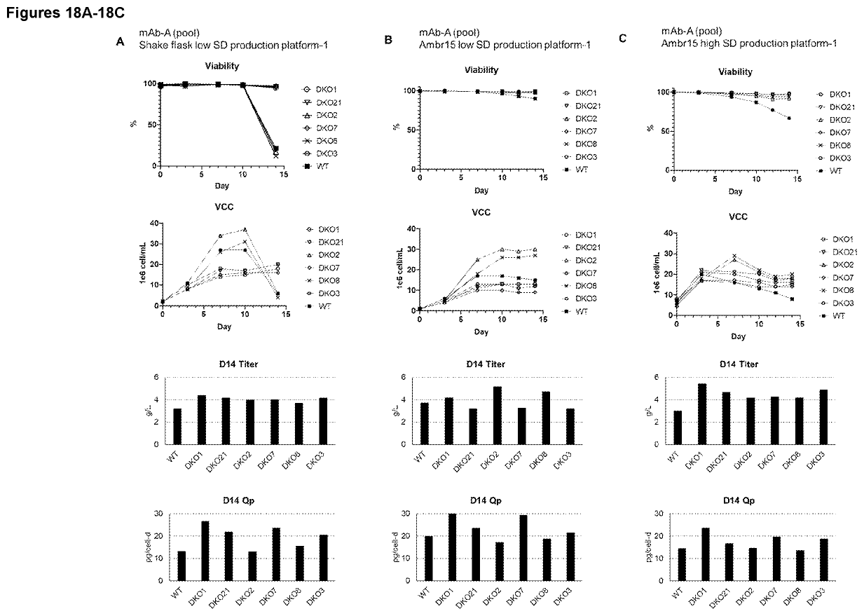

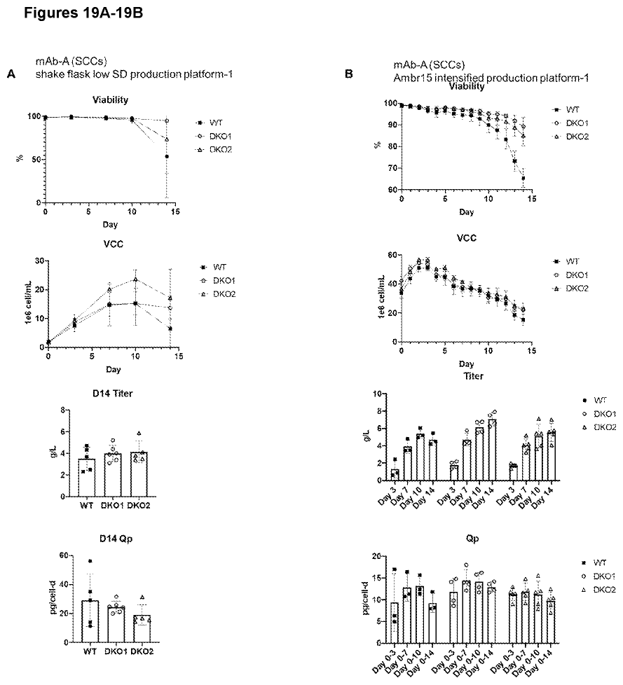

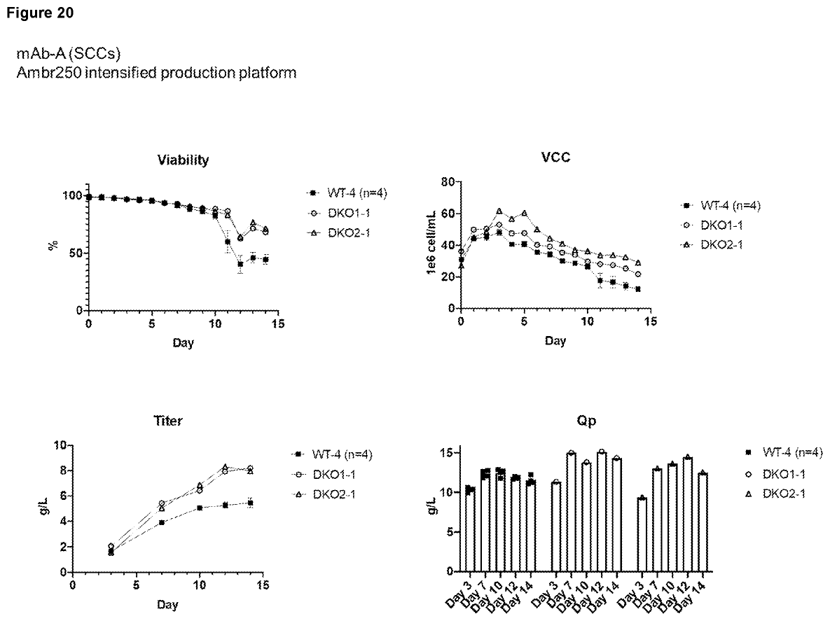

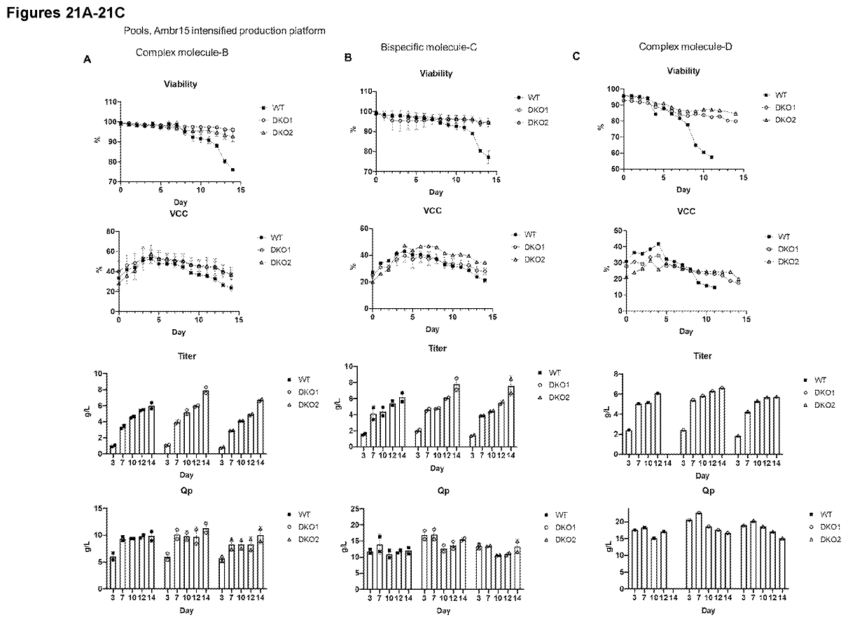

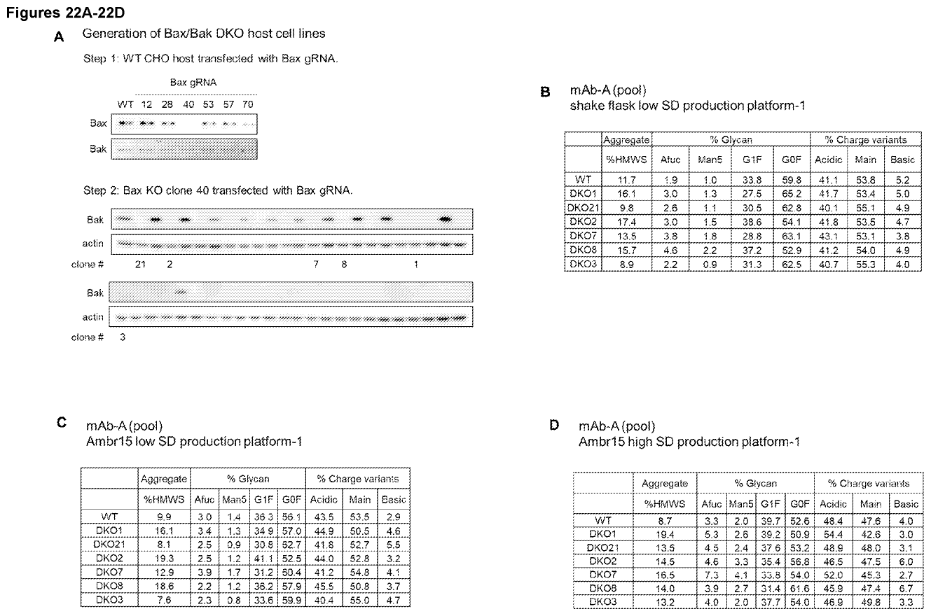

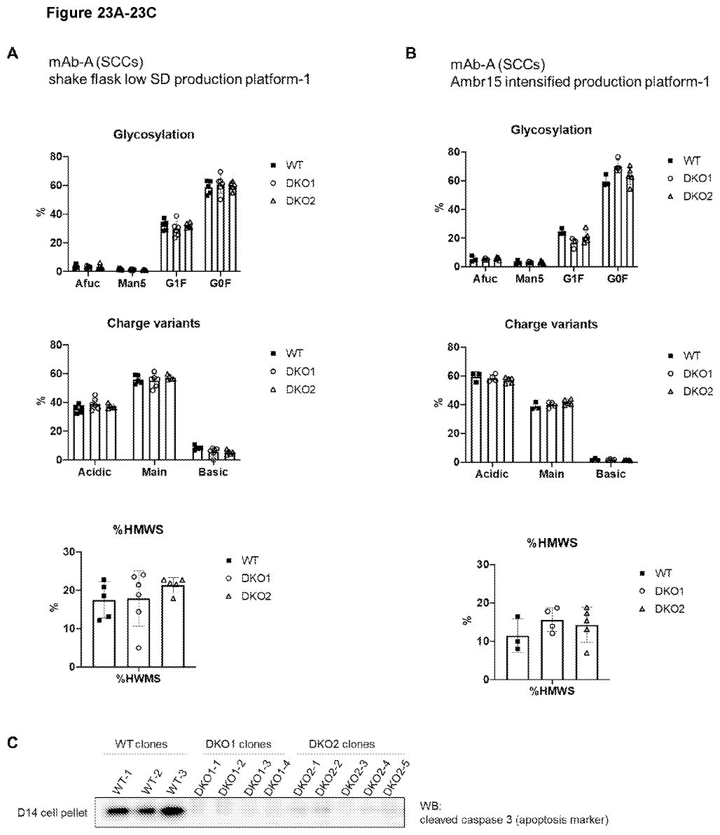

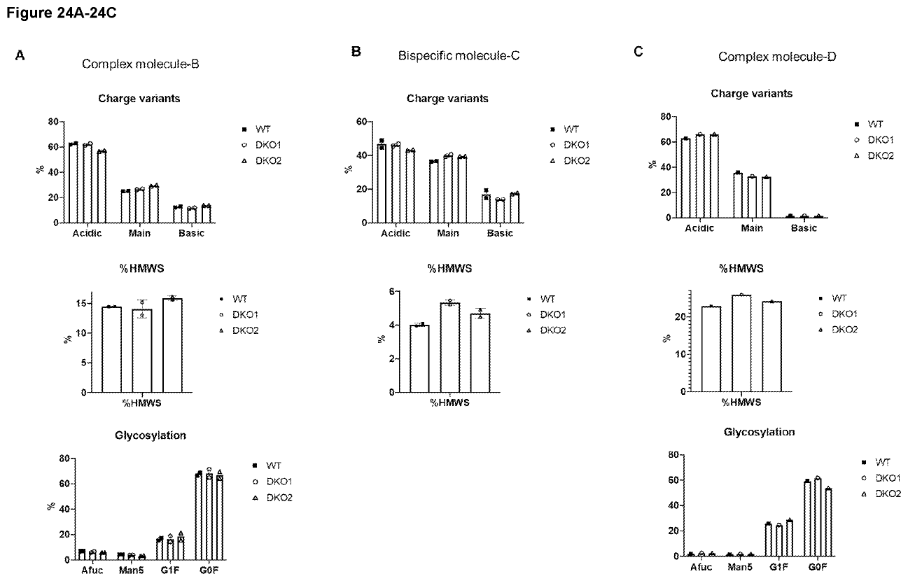

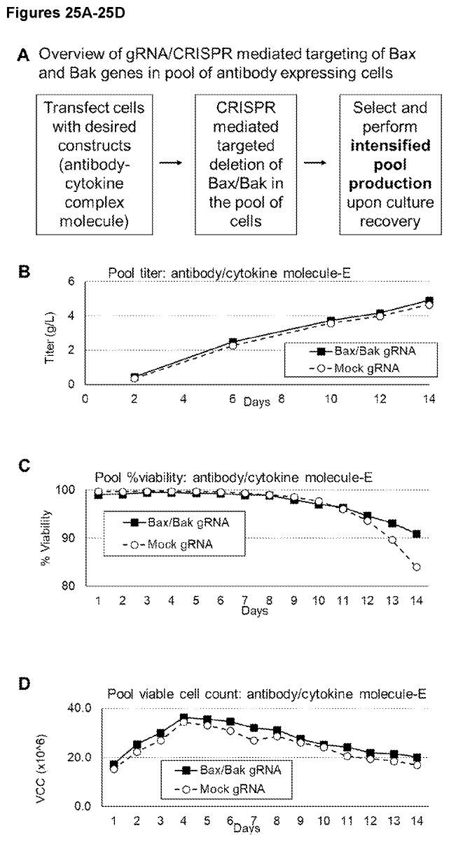

Charge Variants (%): Acidic, Main Peak, Basic. FIGS. 15, 16, and 17 illustrate the percent acidics, main and basics, respectively. The charge variant data is comparable between WT and Bax/Bak DKO clones.

HILIC Glycan Assay (%). Day 14 harvested cell culture fluid (HCCF) was submitted to AO for glycan assay. Table 3 provides an overview of the major glycan species analyzed. The glycan species levels were overall comparable between the WT and Bax/Bak DKO clones. Results obtained from FIGS. 12-17 and Table 3 suggest that antibodies produced from Bax/Bak DKO hosts have comparable product qualities to the ones produced from the WT host.

TABLE 3
Day 14 major glycan species.

| | % Afu | % G0F-N | % G0 | % G0F | % M5 | % G1F | % G2F |
|---|---|---|---|---|---|---|---|
| WT-1 | 4.8 | 0.9 | 1.1 | 56.9 | 2.7 | 26.6 | 5.7 |
| WT-2 | 7.7 | 2.0 | 1.7 | 57.7 | 4.4 | 23.0 | 4.7 |
| WT-3 | 2.5 | 0.9 | 0.7 | 44.0 | 1.1 | 35.7 | 9.9 |
| WT-4 | 3.6 | 1.2 | 0.8 | 64.4 | 2.0 | 23.2 | 4.1 |
| DKO-1-2 | 6.1 | 2.3 | 1.3 | 67.1 | 3.7 | 17.7 | 3.0 |
| DKO-1-3 | 5.0 | 1.9 | 1.1 | 69.1 | 2.7 | 17.6 | 2.8 |
| DKO-1-4 | 4.6 | 2.0 | 1.1 | 76.7 | 2.6 | 12.4 | 1.5 |
| DKO-1-5 | 4.6 | 1.6 | 1.0 | 67.0 | 2.6 | 19.5 | 3.7 |
| DKO-2-1 | 5.5 | 1.9 | 1.3 | 62.6 | 3.1 | 21.6 | 4.1 |
| DKO-2-2 | 6.9 | 2.0 | 1.4 | 63.0 | 4.3 | 20.1 | 4.0 |
| DKO-2-3 | 6.5 | 2.0 | 1.9 | 54.2 | 2.3 | 27.7 | 5.5 |
| DKO-2-4 | 4.4 | 1.8 | 0.9 | 70.7 | 2.8 | 17.0 | 3.1 |
| DKO-2-5 | 6.2 | 2.1 | 1.8 | 66.8 | 3.2 | 18.3 | 3.4 |

### CONCLUSIONS

A higher titer process will not only reduce cost, but also enable the manufacturing network to be more flexible. However, strategies like extending production culture duration, increasing cell density or improving Qp using HDAC inhibitors are hampered by inducing apoptosis in the cell and thus reducing VCC. Using apoptosis resistant host cell lines can diminish this undesired effect in these strategies. In this example, Bax/Bak DKO apoptosis resistant hosts were tested in an extended intensified process. Antibody A producing clones generated from the Bax/Bak DKO hosts exhibited not only improved viability relative to WT cell lines, but also extended productivity in the later stage of the 14-day intensified process. Without wishing to be bound by any theory, the extended productivity of Bax/Bak DKO clones may be due to: 1) knocking out Bax and Bak gene helps maintain mitochondria integrity and health in the later stage of the production, and 2) also prevents/delays apoptosis in culture. In this process, the top Bax/Bak DKO clone generated 50% more antibodies relative to the top WT clone. With further modification of the feeding strategy, it is possible to further increase titer. The product qualities were comparable between Bax/Bak DKO and WT clones. Bax/Bak DKO clones also had similar metabolism to the WT clones

### Example 2: Generation and Testing of Apoptosis Resistant Cells

To further define the benefits of Bax/Bak deficiency in therapeutic molecule manufacturing, the instant example evaluated the production of both a standard monoclonal antibody molecule and several complex molecules in the Bax/Bak DKO genetic background, in regular or intensified processes and at different scales.

### Materials and Methods

### Cell Culture

CHO cells were cultured in a proprietary DMEM/F12-based medium in 125 mL shake flask vessels at 150 rpm, 37° C., and 5% CO2. Cells were passaged at a seeding density of 4×10⁵ cells/mL every 3-4 days.

### Antibody Expressing Cell Line Development

Pool of cells that stably express mAb molecules were generated as described in Misaghi et al., Biotechnol Prog 2013, 29, 727. Expression plasmids were transfected into WT or Bax/Bak DKO CHO cells by MaxCyte STX electroporation (MaxCyte, Gaithersburg, MD). Transfected cells were then selected and expression of mAb was confirmed by FACS via human IgG staining.

### Fed-Batch Production Assay

Fed-batch production cultures were performed in shake flasks, AMBR15 or AMBR250 bioreactors (TAP Biosystems) with proprietary chemically defined production media. For standard or low seeding density processes, cells were seeded at 2×10⁶ cells/ml on Day 0 of the production (N) stage. Cultures received proprietary feed medium on Days 3, 7, and 10. For intensified processes, cells were seeded at 3×10⁷ cells/mL on Day 0 of the production (N) in AMBR15 or AMBR250 vessels. Cultures received proprietary feed media every 2-4 days. Production in the AMBR15 system were operated at set points of 37° C., DO 30%, pH 7.2, and an agitation rate of 1400 rpm. Production in AMBR250 system were operated at set points of 35° C., DO 30%, pH 7.2, and an agitation rate of 477 rpm.

### Bax and Bak Gene Knock-Out

To knock out Bax and Bak genes in CHO cells, gene-targeting Alt-R® crRNA and non-specific Alt-R® tracrRNA from Integrated DNA Technologies, Inc. were reconstituted at 100 μM in Nuclease-Free Duplex Buffer and mixed at 1:1 ratio, followed by incubation at 95° C. for 5 min and cooling down to room temperature to allow annealing. Guide RNA-Cas9 ribonucleoprotein (RNP) complexes were then prepared by mixing 3 μL (150 μmol) annealed gRNA with 1 μL Cas9 protein (IDT, 10 mg/mL) followed by incubation at room temperature for 10 min. Bax and Bak genes were sequentially targeted by transfection of 5 μL gRNA-Cas9 RNP into 1 million cells of a Genentech CHO-K1 host cell line using Neon Electroporation System (Thermo Fisher Scientific), followed by single cell cloning to isolate individual Bax/Bak DKO host cell lines. The complete knock-out of Bax and Bak genes in these DKO host cells lines were confirmed by Western blotting.

Sequences of gRNA oligonucleotides:
Bax gRNA:
GGGTCGGGGGAGCAGCTCGG
Bak gRNA-1:
TCATCACAGTCCTGCCTAGG
Bak gRNA-2:
ATGGCGTCTGGACAAGGACC.

### Off-Line Sample Analyses

Supernatant samples were assayed every other day for viable cell count (VCC), and viability using the Vi-Cell XR (Beckman Coulter), and for pO₂, pH, pCO₂, Na⁺, glucose, and lactate using the Bioprofile 400 (Nova Biomedical). All samples from AMBR bioreactors were analyzed on BioProfile 400 within a few minutes after sampling to minimize off-gassing. The same Vi-Cell XR, BioProfile 400, and osmometer (Model 2020, Advanced Instruments) were used for all samples to eliminate instrument-to-instrument variability. Amino acid concentrations in the supernatant were measured by pre-column derivatization and reversed-phase high performance liquid chromatography. All amino acids were derivatized with 6-Aminoquinolyl-N-Hydroxysuccinimidyl Carbamate (AQC) to produce highly fluorescent derivatives. Antibody titer was measured using high pressure liquid chromatography (HPLC) with a protein A column. Antibody product quality assays were conducted using cell culture supernatant samples purified by PhyTip protein A column. Antibody glycan distribution was analyzed by capillary electrophoresis (CE) with fluorescence detection while molecular size distribution was analyzed by size-exclusion chromatography (SEC). Protein charge heterogeneity was measured using imaged capillary isoelectric focusing (icIEF); all charge heterogeneity samples were pretreated with carboxypeptidase B. All protein product quality assays were developed in-house, and detailed protocols have been published, e.g., Hopp et al., Biotechnol Prog 2009, 25, 1427.

### Results

Pools Generated from Bax/Bak Double-Knock-Out Hosts Resulted in Improved Viability and Higher Titer of a Standard mAb

By targeting Bax and Bak genes sequentially in a Genentech CHO-K1 host cell line (Misaghi et al., Biotechnol Prog 2013, 29, 727) with ribonucleoprotein (RNP) transfection, we were able to generate several different single-cell-cloned Bax/Bak DKO host cell lines whose Bax/Bak deficiency was confirmed by Western blot (Supplementary FIG. 1A). To test whether these DKO host cell lines achieve better viability and recombinant protein expression compared to the parental WT host cell line, we transfected each DKO as well as the WT host cell line to generate stable pools expressing a monoclonal antibody (mAb-A). These pools were first evaluated for their cell culture performance and mAb-A productivity in a 14-day low seeding density production platform (platform-1) in shake flasks (FIG. 1A) as well as in the AMBR15 bioreactors, in which temperature, pH and oxygen levels were continuously monitored and tightly controlled (FIG. 1). In shake flasks, several pools that had better cell growth in the beginning (WT, DKO2, DKO8) exhibited reduced viability in the end of the process, possibly due to lack of process control, e.g. pH control or gassing. In contrast, in AMBR15 bioreactors, all of the pools showed good viability throughout the process with only WT pool showing a slight decrease in viability at the end of the culture process. In both shake flasks and AMBR15 processes, on average, pools derived from the DKO hosts achieved slightly better or comparable titers and specific productivities relative to the WT pool (FIGS. 1A and 1B).

To determine whether Bax/Bak DKO cells achieve better viability and productivity in intensified processes, the pools were then tested in a high seeding density process in AMBR15 bioreactors (FIG. 1C). In the high seeding density process, the WT pool showed declining viability from day 3 and had only 67% viable cells at the end of the process, while all the DKO pools maintained over 90% viability throughout the process. All DKO pools achieved higher titers than the WT pool in the intensified process, mainly because of better viabilities and higher viable cell counts (VCCs). These results suggest that the Bax/Bak DKO genetic modification prevented cell death in the intensified processes and therefore led to higher titer.

In both shake flask and AMBR15 production processes no drastic differences in product quality attributes between WT and DKO pools were observed (Supplementary FIG. 1B-D). In the intensified process, there were higher percentage of high molecular weight species (HMWS) or protein aggregates observed in the products generated from the DKO pools (Supplementary FIG. 1D), which is commonly associated with higher titer. All these cell lines were comparable with regard to other product quality attributes such as charge variant or glycosylation levels (Supplementary FIG. 1B-D).

Single Cell Clones Generated from Bax/Bak Double-Knock-Out Hosts Enabled Process Extension by Improved Cell Culture Viability Resulting in Higher Titer of Standard mAb in an Intensified Process

To determine whether the benefit of achieving better viability and mAb-A titer observed in the Bax/Bak DKO pools could be maintained after single cell cloning, we single cell cloned the WT pool as well as two Bax/Bak DKO pools and picked the top 4-5 clones from each arm after two rounds of in plate titer assays. The single cell clones (SCCs) were first analyzed in the low cell seeding density fed-batch production process in shake flasks (FIG. 2A). Similar to the pool results, there were improvements in cell culture viability and a slight increase in titer of the DKO clones compared to the WT clone. All the product quality attributes were comparable between WT clone and DKO clones (Supplementary FIG. 2A).

Whether Bax/Bak DKO clones would achieve higher titer in the intensified process was tested. The top clones from the WT and two DKO arms were tested in a prolonged (14 days) intensified platform-1 fed-batch production process with a targeted 30×10⁶ cell/mL starting cell seeding density in AMBR15 bioreactors (FIG. 2B). Unlike WT clones that had decreased viability in the later days of the process, the DKO clones maintained high viability until the end of the 14-day process (FIG. 2B). The levels of an apoptosis marker protein, cleaved caspase 3, were elevated in the WT but not DKO clones on Day 14, indicating that WT cells were undergoing apoptosis in the later stages of the intensified production process, while knocking out Bax/Bak genes prevented it (Supplementary FIG. 2C). Furthermore, the titers of WT clone plateaued around Day 10 and reached 4.7±0.7 g/L on Day 14, while the titers of DKO clones kept increasing and achieved 7.1±0.8 g/L on Day 14. Note that the titer decrease from Day 10 to 14 for WT clones and the slower titer increase for DKO clones were due to dilution of the production culture by removing samples from the culture and adding feed and glucose back into the culture. The reduced mAb-A productivity of WT clones in the last 4 days of the process was not only due to loss of viability and VCC, but also a reduced specific productivity (Qp). On average, WT clones had lower overall Qp in the whole 14 days of the process (FIG. 2B, lowest panel, Day 0-14) than in the first 10 days (FIG. 2B, lowest panel, Day 0-10), indicating a reduced Qp in the last 4 days for WT clones. The lower Qp of WT clones versus the DKO clones in the final stage of the process could be due to mitochondrial membrane damage from Bax/Bak activation in the still viable cells. These results indicate that Bax/Bak DKO cells enable extended cell culture viability and thus an extended production process in an intensified process, resulting in a 44% titer increase compared to the WT clones in the best case. No significant product quality change was observed in the DKO cells (Supplementary FIG. 2B) relative to the WT cells.

To evaluate whether production scale affects the performance of DKO cells, the top clone from each host in AMBR250 vessels in the same intensified process was tested. At this larger scale, DKO clones achieved better viabilities, Qp, and titers (8.2 g/L) than the WT clone (5.5±0.4 g/L, 4 replicates from the same clone) (FIG. 3), similar to the AMBR15 process.

### Bax/Bak Double-Knock-Out Also Improves Complex Antibody Production by Improving Viability in an Intensified Process

Compared to standard mAb molecules, the production of bispecific or complex antibodies is more challenging due to their non-standard format, which can pose additional manufacturing problems such as product instability, undesired byproduct species, higher product fragment or aggregation levels, and low expression levels. The non-natural format of bispecific antibodies or complex molecules increases the chance of molecule misfolding and disulfide bond mispairing, causing higher levels of intracellular reactive oxygen species (ROS) accumulation and oxidative stress inside the cell, which eventually leads to low VCD, viability and productivity. Whether Bax/Bak DKO cell lines can help to mitigate these issues was tested.

The production of two complex molecules (B and D) and one bispecific molecule (C) in either WT host or DKO hosts were compared (FIG. 4). For complex molecule-B and bispecific molecule-C, two stable expression pools from each host were generated, while for complex molecule-D, one stable expression pool from each host was generated. These pools were tested in a prolonged (14-day) intensified production process in AMBR15 bioreactors. For all three molecules, pools that were generated from the DKO hosts remained at high viabilities throughout the process while the WT pools showed decreased viability at the later stage of the production (FIG. 4). Better viabilities also led to higher VCCs of the DKO pools, resulting in about 30% higher yields for molecule-B and molecule-C (7.9±0.5 and 7.8±1.0 g/L respectively) in DKO1 host compared to the WT (6.0±0.5 g/L and 6.2±0.8 g/L respectively) (FIGS. 4B and 4C). For molecule-D, DKO pools only achieved 5% higher titer relative to the WT pool, mainly due to decreased specific productivity of the DKO pools at later stages of production, likely triggered by depletion of essential amino acids, such as cysteine, in culture (FIG. 4C). All the product quality attributes were comparable between WT and DKO pools for all three molecules (Supplementary FIG. 3). Overall, Bax/Bak DKO cell lines expressing bispecific or complex molecules maintained high viability in a 14-day intensified production process, leading to higher product titer without impacting product quality.

Knocking out Bax/Bak from an established therapeutic protein expressing cell line also showed some beneficial traits even at the pool stage. As shown in Supplementary FIG. 4, we transfected either mock or Bax/Bak gRNAs into a pool of cells previously transfected with an antibody-cytokine (complex molecule-E) expressing construct. The cell culture performances and titers of the control and Bax/Bak DKO pools were compared in the intensified production process in AMBR250 bioreactors. Similar to previous results, deletion of Bax/Bak genes improved culture viability in the later stage of the production, however, as the knock-out efficiency of Bax and Bak genes were less than 50% (data not shown), little improvements in VCC and titer in the DKO pool was observed. We believe that by optimizing and improving gene knock-out efficiency, transfection of Bax/Bak gRNA into an established cell line or recently transfected pool of cells, followed by SCC, can also enable isolation of single cell clones with complete Bax/Bak DKO phenotype capable of achieve high viability and titer in the intensified production process.

### DISCUSSION

In this example, Bax/Bak DKO apoptosis resistant host cell lines were generated and expressed both standard monoclonal antibodies and several complex molecules in intensified production processes as pools and single cell clones, and at different scales. Therapeutic protein expressing pools or clones generated from the Bax/Bak DKO hosts exhibited extended viability and productivity while both viability and productivity were reduced in the later stage of the intensified production process when the WT host was employed. Meanwhile, the product quality attributes were comparable between Bax/Bak DKO and WT cell lines. Overall, the data indicates that the utilization of apoptosis-resistant host cell lines significantly improved the process intensification strategy, yielding a higher volumetric productivity without altering the product qualities and thus enabling a prolonged production process. Knocking out both Bax and Bak genes helped to maintain high viability throughout a 14-day production process in all cases. Even by transfecting Bax/Bak gRNA into a cell line previously transfected with constructs expressing a complex molecule, hence generating a heterogenous pool (Supplementary FIG. 4), helped to improve viability. The high culture viability itself is very beneficial to the manufacturing processes, since it allows better control of product quality. Besides viability improvement, in most of the intensified production processes, the culture titers were increased by 30-80%.

Less titer improvement was observed at regular or lower seeding densities (FIGS. 1A and 2A) relative to the intensified process, mainly because of lower cell death in the control cultures. Apoptosis happens more frequently during the intensified production processes when the cell density is high, likely due to elevated risk of hypoxia, shear stress, nutrient deprivation, and faster accumulation of toxic metabolic by-products produced by the cells including inhibitory metabolites (e.g. isovalerate and formic acid) and reactive oxygen species (ROS). Perfusion techniques can be used to reduce these cellular stresses by reducing intracellular ROS accumulation, and removing inhibitory metabolites from the cell culture while continuously providing the culture with oxygen and nutrients. However, because of the complex process control and large volumes of media required, perfusion cell culture is generally less ideal. The utilization of apoptosis-resistant Bax/Bak DKO cell lines in this study provides an alternative approach to increase culture cell density and improve titer in the intensified fed-batch cultures. Another way to mitigate the extensive culture viability drop during the fed-batch intensified production process is to shorten the fed-batch cultivation time and harvest the culture before the viability starts to decline. However, the shortened cultivation time reduces economic benefit and improved manufacturing network flexibility because of the massive cost of materials and labor that are required to set up the production cultures. Therefore, extending the cultivation time while maintaining culture productivity is important to achieve the desired benefit-cost ratio and increase manufacturing flexibility. As shown in this example, by using Bax/Bak DKO cell lines the production cultivation time can be extended to at least 14 days compared to 7-10 days with the WT cells, and permits a 30-50% titer increase with both standard antibodies and complex molecules, which will significantly reduce the production cost per unit of product. In summary, the Bax/Bak apoptotic resistant CHO cells allow for a high-viability/high-yield intensified production process with high cell density and extended cultivation time.

What is claimed is:

1. An isolated eukaryotic cell line, wherein the cell line comprises a stable integrated loss-of-function or attenuation-of-function mutation in each of the Bax and Bak genes.

2. The cell line of claim 1, wherein the cell line comprises a stable integrated loss-of-function mutation in each of the Bax and Bak genes.

3. The cell line of claim 1 or claim 2, wherein the cell line is an animal cell line or a fungal cell line.

4. The cell line of claim 3, wherein the animal cell line is a mammalian cell line.

5. The cell line of claim 4, wherein the mammalian cell line is a COS cell line, a VERO cell line, a HeLa cell line, a HEK 293 cell line, a PER-C6 cell line, a K562 cell line, a MOLT-4 cell line, a Ml cell line, a NS-1 cell line, a COS-7 cell line, a MDBK cell line, a MDCK cell line, a MRC-5 cell line, a WI-38 cell line, a WEHI cell line, a SP2/0 cell line, a BHK cell line or a CHO cell line, or their derivatives.

6. The cell line of claim 5, wherein the CHO cell line is a CHO K1 cell line, a CHO K1SV cell line, a DG44 cell line, a DUKXB-11 cell line, a CHOK1S cell line, or a CHO K1M cell line, or their derivatives.

7. The cell line of any preceding claim, wherein the cell line comprises a deletion in each of the Bax and Bak genes.

8. The cell line of any preceding claim, wherein the cell line further comprises a viral genome and one or more polynucleotides encoding a viral capsid.

9. The cell line of any preceding claim, wherein the cell line further comprises a polynucleotide encoding a product of interest.

10. The cell line of claim 9, wherein the polynucleotide that encodes the product of interest is integrated in the cellular genome of the cell line at a targeted location.

11. The cell line of claim 9, wherein the polynucleotide that encodes the product of interest is randomly integrated in the cellular genome of the cell line.

12. The cell line of any of claims 9-11, wherein the polynucleotide that encodes the product of interest is an extrachromosomal polynucleotide.

13. The cell line of any of claims 9-11, wherein the polynucleotide that encodes the product of interest is integrated into a chromosome of the cell line.

14. The cell line of any of claims 9-11, wherein product of interest comprises a recombinant polypeptide.

15. The cell line of any of claims 9-14, wherein the product of interest comprises an antibody, an antibody-fusion protein, an antigen, an enzyme, or a vaccine.

16. The cell line of claim 15, wherein the antibody is a multispecific antibody or antigen-binding fragment thereof.

17. The cell line of claim 15 or claim 16, wherein the antibody consists of a single heavy chain sequence and a single light chain sequence or antigen-binding fragments thereof.

18. The cell line of any of claims 15-17, wherein the antibody comprises a chimeric antibody, a human antibody or a humanized antibody.

19. The cell line of any of claims 15-18, wherein the antibody comprises a monoclonal antibody.

20. The cell line of any of claims 7-19, wherein the cell line has a higher specific productivity than a corresponding isolated eukaryotic cell line that comprises the polynucleotide and functional copies of each of the wild type Bax and Bak genes.

21. The cell line of any preceding claim, wherein the cell line is more resistant to apoptosis than a corresponding isolated eukaryotic cell line that comprises functional copies of each of the Bax and Bak genes.

22. The cell line of any preceding claim, wherein the cell line is employed in cell culture processes such as fed-batch, perfusion, process intensified, semi-continuous perfusion, or continuous perfusion.

23. The cell line of claim 22, wherein the cell line is employed in an intensified perfusion process.

24. A composition comprising a eukaryotic cell line according to any preceding claim.

25. The composition of claim 24, further comprising a cell culture medium.

26. A cell culture comprising a cell culture medium and a plurality of eukaryotic cells, wherein each cell of the plurality comprises a stable integrated loss-of-function or attenuation-of-function mutation in each of the Bax and Bak genes.

27. The cell culture of claim 26, wherein each cell comprises a stable integrated loss-of-function mutation in each of the Bax and Bak genes.

28. The cell culture of claim 26 or claim 27, wherein each cell of the plurality comprises a deletion in each of the Bax and Bak genes.

29. The cell culture of any of claims 26-28, wherein the cells are animal cells or fungal cells.

30. The cell culture of claim 29, wherein the animal cells are mammalian cells.

31. The cell culture of claim 30, wherein the mammalian cells are COS cells, VERO cells, HeLa cells, HEK 293 cells, PER-C6 cells, K562 cells, MOLT-4 cells, Ml cells, NS-1 cells, COS-7 cells, MDBK cells, MDCK cells, MRC-5 cells, WI-38 cells, WEHI cells, SP2/0 cells, BHK cells or a CHO cells, or their derivatives.

32. The cell culture of claim 31, wherein the CHO cells are a CHO K1 cells, CHO K1SV cells, DG44 cells, DUKXB-11 cells, CHOK1S cells, or CHO K1M cells, or their derivatives.

33. The cell culture of any of claims 26-32, wherein the cell culture further comprises a polynucleotide that encodes a product of interest.

34. The cell culture of claim 33, wherein the polynucleotide that encodes the product of interest is integrated in the cellular genome of the cells at a targeted location.

35. The cell culture of claim 33, wherein the polynucleotide that encodes the product of interest is randomly integrated in the cellular genome of the cells.

36. The cell culture of any of claims 33-35, wherein the polynucleotide that encodes the product of interest is an extrachromosomal polynucleotide.

37. The cell culture of any of claims 33-35, wherein the polynucleotide that encodes the product of interest is integrated into a chromosome of the cells.

38. The cell culture of any of claims 33-37, wherein the product of interest comprises a recombinant polypeptide.

39. The cell culture of any of claims 33-38, wherein the product of interest is an antibody, an antibody-fusion protein, an antigen, an enzyme, or a vaccine.

40. The cell culture of claim 39, wherein the antibody is a multispecific antibody or antigen-binding fragment thereof.

41. The cell culture of claim 39 or claim 40, wherein the antibody consists of a single heavy chain sequence and a single light chain sequence or antigen-binding fragments thereof.

42. The cell culture of any of claims 39-41, wherein the antibody comprises a chimeric antibody, a human antibody or a humanized antibody.

43. The cell culture of any of claims 39-42, wherein the antibody comprises a monoclonal antibody.

44. The cell culture of any of claims 26-43, wherein the each of the cells further comprise a recombinant polynucleotide.

45. The cell culture of claims 26-44, wherein the cells are employed in a cell culture process such as fed-batch, perfusion, process intensified, semi-continuous perfusion, or continuous perfusion.

46. The cell culture of claim 45, wherein the cells are employed in an intensified perfusion process.

47. A method of reducing apoptotic activity in a eukaryotic cell, comprising administering to the cell a genetic engineering system, wherein the genetic engineering system:
a. knocks down or knocks out the expression of a Bax polypeptide isoform; and
b. knocks down or knocks out the expression of a Bak polypeptide isoform.

48. The method of claim 47, wherein the method further comprises employing the eukaryotic cell in a fed-batch, perfusion, process intensified, semi-continuous perfusion, or continuous perfusion cell culture process.

49. The method of claim 48, wherein the eukaryotic cell is employed in an intensified cell culture process.

50. The method of any of claims 47-49, wherein the genetic engineering system is selected from the group consisting of a CRISPR/Cas system, a zinc-finger nuclease (ZFN) system, a transcription activator-like effector nuclease (TALEN) system and a combination thereof.

51. The method of any of claims 47-50, wherein the genetic engineering system is or comprises a CRISPR/Cas9 system.

52. The method of claim 51, wherein the CRISPR/Cas9 system comprises:
a. a Cas9 molecule,
b. at least one first guide RNA (gRNA) comprising a targeting sequence that is complementary to a target sequence in a Bax gene, and
c. at least one second gRNA comprising a targeting sequence that is complementary to a target sequence in a Bak gene.

53. The method of claim 52, wherein at least one of the target sequences is a portion of the Bax gene, and/or wherein at least one of the target sequences is a portion of the Bak gene.

54. The method of any of claims 47-53, wherein the expression of the Bax polypeptide and/or the expression of the Bak polypeptide is knocked out, and the apoptotic activity of the cell is reduced compared to the apoptotic activity of a reference cell.

55. The method of any of claims 47-53, wherein the expression of the Bax polypeptide and/or the expression of the Bak polypeptide is knocked down, and the apoptotic activity of the cell is reduced compared to the apoptotic activity of a reference cell.

56. The method of claim 54 or claim 55, wherein the apoptotic activity of the cell is determined from the viability for a population of said cells compared to the viability of a population of said reference cells determined at day 14 of a production phase.

57. The method of any of claims 54-56, wherein the reference cell is a cell that comprises wild-type alleles of the Bax and Bak genes.

58. The method of any of claims 47-57, wherein the genetic engineering system is or comprises a zinc-finger nuclease (ZFN) system or a transcription activator-like effector nuclease (TALEN) system.

59. The method of any of claims 47-58, wherein the cell line development system comprises targeted integration, random integration or transposase systems.

60. The method of any of claims 47-59, wherein the cell is an animal cell or a fungal cell.

61. The method of claim 60, wherein the animal cell is a mammalian cell.

62. The method of claim 61, wherein the mammalian cell is a COS cell, a VERO cell, a HeLa cell, a HEK 293 cell, a PER-C6 cell, a K562 cell, a MOLT-4 cell, a Ml cell, NS-1 cell, a COS-7 cell, a MDBK cell, a MDCK cell, a MRC-5 cell, a WI-38 cell, a WEHI cell, a SP2/0 cell line, a BHK cell or a CHO cell line, or their derivatives.

63. The method of claim 62, wherein the CHO cell is a CHO K1 cell, a CHO K1SV cell, a DG44 cell, a DUKXB-11 cell, a CHOK1S cell, or a CHO K1M cell, or their derivatives.

64. The method of any of claims 47-63, wherein the cell further comprises a polynucleotide that encodes a product of interest.

65. The method of claim 64, wherein the polynucleotide that encodes the product of interest is integrated in the cellular genome of the cell at a targeted location.

66. The method of claim 64, wherein the polynucleotide that encodes the product of interest is randomly integrated in the cellular genome of the cell.

67. The method of any of claims 64-66, wherein the polynucleotide that encodes the product of interest is an extrachromosomal polynucleotide.

68. The method of any of claims 64-66, wherein the polynucleotide that encodes the product of interest is integrated into a chromosome of the cell.

69. The method of any of claims 64-68, wherein the product of interest comprises a recombinant polypeptide.

70. The method of any of claims 64-69, wherein the product of interest is an antibody, an antibody-fusion protein, an antigen, an enzyme, or a vaccine.

71. The method of claim 70, wherein the antibody is a multispecific antibody or antigen-binding fragment thereof.

72. The method of claim 70 or claim 71, wherein the antibody consists of a single heavy chain sequence and a single light chain sequence or antigen-binding fragments thereof.

73. The method of any of claims 70-72, wherein the antibody comprises a chimeric antibody, a human antibody or a humanized antibody.

74. The method of any of claims 70-73, wherein the antibody comprises a monoclonal antibody.

75. The method of any of claims 70-74, wherein the each of the cells further comprise a recombinant polynucleotide.

76. A method of producing a recombinant polypeptide, comprising:
culturing a eukaryotic cell line that comprises:
(a) a stable integrated loss-of-function or attenuation-of-function mutation in each of the Bax and Bak genes, and
(b) a polynucleotide encoding the recombinant polypeptide,
under conditions suitable for production of the polypeptide.

77. The method of claim 76, wherein the polynucleotide that encodes the polypeptide is integrated in the cellular genome of the cells of the cell line at a targeted location.

78. The method of claim 76, wherein the polynucleotide that encodes the polypeptide is randomly integrated in the cellular genome of the cells of the cell line.

79. The method of any of claims 76-78, wherein the polynucleotide that encodes the polypeptide is an extrachromosomal polynucleotide.

80. The method of any of claims 76-79, wherein the polynucleotide that encodes the polypeptide is integrated into a chromosome of the cells of the cell line.

81. The method of any of claims 76-80, wherein the recombinant polypeptide is an antibody, an antibody-fusion protein, an antigen, an enzyme, or a vaccine.

82. The method of claim 81, wherein the antibody is a multispecific antibody or antigen-binding fragment thereof.

83. The method of claim 81 or claim 82, wherein the antibody consists of a single heavy chain sequence and a single light chain sequence or antigen-binding fragments thereof.

84. The method of any of claims 81-83, wherein the antibody comprises a chimeric antibody, a human antibody or a humanized antibody.

85. The method of any of claims 81-84, wherein the antibody comprises a monoclonal antibody.

86. The method of any of claims 76-85, further comprising isolating the recombinant polypeptide.

87. A method of producing a viral vector, comprising:
culturing a eukaryotic cell line that comprises (a) stable integrated a loss-of-function or attenuation-of function mutation in each of the Bax and Bak genes, (b) a viral genome, and (c) one or more polynucleotides encoding a viral capsid, under conditions suitable for production of the viral vector.

88. The method of claim 87, further comprising isolating the viral vector.

89. The method of any of claims 87-88, wherein the cell line is an animal cell line, or a fungal cell line.

90. The method of claim 89, wherein the animal cell line is a mammalian cell line.

91. The method of claim 90, wherein the mammalian cell line is a COS cell line, a VERO cell line, a HeLa cell line, a HEK 293 cell line, a PER-C6 cell line, a K562 cell line, a MOLT-4 cell line, a Ml cell line, a NS-1 cell line, a COS-7 cell line, a MDBK cell line, a MDCK cell line, a MRC-5 cell line, a WI-38 cell line, a WEHI cell line, a SP2/0 cell line, a BHK cell line or a CHO cell line, or their derivatives.

92. The method of claim 91, wherein the CHO cell line is a CHO K1 cell line, a CHO K1SV cell line, a DG44 cell line, a DUKXB-11 cell line, a CHOK1S cell line, or a CHO K1M cell line, or their derivatives.

93. The method of any of claims 87-92, wherein the cell line is cultured in a cell culture medium.

94. The method of any of claims 87-93, wherein the cell line is cultured under fed-batch culture conditions, or perfusion culture conditions.

95. The method of claim 94, wherein the cell line is cultured under fed-batch culture conditions, optionally wherein the fed-batch culture conditions are intensified fed-batch culture conditions.

96. The method of any of claims 87-95, wherein the cell line is cultured under perfusion culture conditions, optionally wherein the perfusion culture conditions are semi-continuous perfusion or continuous perfusion.

97. The method of any of claims 87-96, wherein the cell line comprises a stable integrated loss-of-function mutation in each of the Bax and Bak genes.


SEQUENCE LISTING (35 sequences)

SEQ ID NO: 1 - protein, 6 aa, synthetic construct
  REGION 1..6: note = source = /note="Description of Artificial Sequence: Synthetic peptide"
SSYYMA

SEQ ID NO: 2 - protein, 5 aa, synthetic construct
  REGION 1..5: note = source = /note="Description of Artificial Sequence: Synthetic peptide"
DSYMS

SEQ ID NO: 3 - protein, 17 aa, synthetic construct
  REGION 1..17: note = source = /note="Description of Artificial Sequence: Synthetic peptide"
DMYPDNGDSSYNQKFRE

SEQ ID NO: 4 - protein, 8 aa, synthetic construct
  REGION 1..8: note = source = /note="Description of Artificial Sequence: Synthetic peptide"
APRWYFSV

SEQ ID NO: 5 - protein, 11 aa, synthetic construct
  REGION 1..11: note = source = /note="Description of Artificial Sequence: Synthetic peptide"
RASQDISNYLN

SEQ ID NO: 6 - protein, 7 aa, synthetic construct
  REGION 1..7: note = source = /note="Description of Artificial Sequence: Synthetic peptide"
YTSRLRS

SEQ ID NO: 7 - protein, 9 aa, synthetic construct
  REGION 1..9: note = source = /note="Description of Artificial Sequence: Synthetic peptide"
QQGHTLPPT

SEQ ID NO: 8 - protein, 117 aa, synthetic construct
  REGION 1..117: note = source = /note="Description of Artificial Sequence: Synthetic polypeptide"
EVQLVQSGAEVKKPGASVKVSCKASGYTFTDSYMSWVRQAPGQGLEWIGDMYPDNGDSSYNQKFRERVTITRDTSTSTAYLELSSLRSEDTAVYYCVLAPRWYFSVWGQGTLVTVSS

SEQ ID NO: 9 - protein, 107 aa, synthetic construct
  REGION 1..107: note = source = /note="Description of Artificial Sequence: Synthetic polypeptide"
DIQMTQSPSSLSASVGDRVTITCRASQDISNYLNWYQQKPGKAPKLLIYYTSRLRSGVPSRFSGSGSGTDFTLTISSLQPEDFATYYCQQGHTLPPTFGQGTKVEIK

SEQ ID NO: 10 - protein, 5 aa, synthetic construct
  REGION 1..5: note = source = /note="Description of Artificial Sequence: Synthetic peptide"
NYLIE

SEQ ID NO: 11 - protein, 17 aa, synthetic construct
  REGION 1..17: note = source = /note="Description of Artificial Sequence: Synthetic peptide"
VINPGSGDTYYSEKFKG

SEQ ID NO: 12 - protein, 5 aa, synthetic construct
  REGION 1..5: note = source = /note="Description of Artificial Sequence: Synthetic peptide"
DRLDY

SEQ ID NO: 13 - protein, 11 aa, synthetic construct
  REGION 1..11: note = source = /note="Description of Artificial Sequence: Synthetic peptide"
HASQDISSYIV

SEQ ID NO: 14 - protein, 7 aa, synthetic construct
  REGION 1..7: note = source = /note="Description of Artificial Sequence: Synthetic peptide"
HGTNLED

SEQ ID NO: 15 - protein, 9 aa, synthetic construct
  REGION 1..9: note = source = /note="Description of Artificial Sequence: Synthetic peptide"
VHYAQFPYT

SEQ ID NO: 16 - protein, 114 aa, synthetic construct
  REGION 1..114: note = source = /note="Description of Artificial Sequence: Synthetic polypeptide"
EVQLVQSGAEVKKPGASVKVSCKASGYAFTNYLIEWVRQAPGQGLEWIGVINPGSGDTYYSEKFKGRVTITRDTSTSTAYLELSSLRSEDTAVYYCARDRLDYWGQGTLVTVSS

SEQ ID NO: 17 - protein, 107 aa, synthetic construct
  REGION 1..107: note = source = /note="Description of Artificial Sequence: Synthetic polypeptide"
DIQMTQSPSSLSASVGDRVTITCHASQDISSYIVWYQQKPGKAPKLLIYHGTNLEDGVPSRFSGSGSGTDFTLTISSLQPEDFATYYCVHYAQFPYTFGQGTKVEIK

SEQ ID NO: 18 - protein, 215 aa, synthetic construct
  REGION 1..215: note = source = /note="Description of Artificial Sequence: Synthetic polypeptide"
DIQMTQSPSSLSASVGDRVTITCKASAAVGTYVAWYQQKPGKAPKLLIYSASYRKRGVPSRFSGSGSGTDFTLTISSLQPEDFATYYCHQYYTYPLFTFGQGTKLEIKRTVAAPSVFIFPPSDEQLKSGTASVVCLLNNFYPREAKVQWKVDNALQSGNSQESVTEQDSKDSTYSLSSTLTLSKADYEKHKVYACEVTHQGLSSPVTKSFNRGEC

SEQ ID NO: 19 - protein, 214 aa, synthetic construct
  REGION 1..214: note = source = /note="Description of Artificial Sequence: Synthetic polypeptide"
QAVVTQEPSLTVSPGGTVTLTCGSSTGAVTTSNYANWVQEKPGQAFRGLIGGTNKRAPGTPARFSGSLLGGKAALTLSGAQPEDEAEYYCALWYSNLWVFGGGTKLTVLSSASTKGPSVFPLAPSSKSTSGGTAALGCLVKDYFPEPVTVSWNSGALTSGVHTFPAVLQSSGLYSLSSVVTVPSSSLGTQTYICNVNHKPSNTKVDKKVEPKSC

SEQ ID NO: 20 - protein, 694 aa, synthetic construct
  REGION 1..694: note = source = /note="Description of Artificial Sequence: Synthetic polypeptide"
QVQLVQSGAEVKKPGASVKVSCKASGYTFTEFGMNWVRQAPGQGLEWMGWINTKTGEATYVEEFKGRVTFTTDTSTSTAYMELRSLRSDDTAVYYCARWDFAYYVEAMDYWGQGTTVTVSSASTKGPSVFPLAPSSKSTSGGTAALGCLVKDYFPEPVTVSWNSGALTSGVHTFPAVLQSSGLYSLSSVVTVPSSSLGTQTYICNVNHKPSNTKVDKKVEPKSCDGGGGSGGGGSEVQLLESGGGLVQPGGSLRLSCAASGFTFSTYAMNWVRQAPGKGLEWVSRIRSKYNNYATYYADSVKGRFTISRDDSKNTLYLQMNSLRAEDTAVYYCVRHGNFGNSYVSWFAYWGQGTLVTVSSASVAAPSVFIFPPSDEQLKSGTASVVCLLNNFYPREAKVQWKVDNALQSGNSQESVTEQDSKDSTYSLSSTLTLSKADYEKHKVYACEVTHQGLSSPVTKSFNRGECDKTHTCPPCPAPEAAGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALGAPIEKTISKAKGQPREPQVYTLPPCRDELTKNQVSLWCLVKGFYPSDIAVEWESNGQPENNYKTTPPVLDSDGSFFLYSKLTVDKSRWQQGNVFSCSVMHEALHNHYTQKSLSLSPGK

SEQ ID NO: 21 - protein, 451 aa, synthetic construct
  REGION 1..451: note = source = /note="Description of Artificial Sequence: Synthetic polypeptide"
QVQLVQSGAEVKKPGASVKVSCKASGYTFTEFGMNWVRQAPGQGLEWMGWINTKTGEATYVEEFKGRVTFTTDTSTSTAYMELRSLRSDDTAVYYCARWDFAYYVEAMDYWGQGTTVTVSSASTKGPSVFPLAPSSKSTSGGTAALGCLVKDYFPEPVTVSWNSGALTSGVHTFPAVLQSSGLYSLSSVVTVPSSSLGTQTYICNVNHKPSNTKVDKKVEPKSCDKTHTCPPCPAPEAAGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALGAPIEKTISKAKGQPREPQVCTLPPSRDELTKNQVSLSCAVKGFYPSDIAVEWESNGQPENNYKTTPPVLDSDGSFFLVSKLTVDKSRWQQGNVFSCSVMHEALHNHYTQKSLSLSPGK

SEQ ID NO: 22 - protein, 453 aa, synthetic construct
  REGION 1..453: note = source = /note="Description of Artificial Sequence: Synthetic polypeptide"
EVQLVESGGGLVQPGGSLRLSCAASGYDFTHYGMNWVRQAPGKGLEWVGWINTYTGEPTYAADFKRRFTFSLDTSKSTAYLQMNSLRAEDTAVYYCAKYPYYYGTSHWYFDVWGQGTLVTVSSASTKGPSVFPLAPSSKSTSGGTAALGCLVKDYFPEPVTVSWNSGALTSGVHTFPAVLQSSGLYSLSSVVTVPSSSLGTQTYICNVNHKPSNTKVDKKVEPKSCDKTHTCPPCPAPEAAGGPSVFLFPPKPKDTLMASRTPEVTCVVVDVSHEDPEVKFNWYVDGVEVHNAKTKPREEQYNSTYRVVSVLTVLAQDWLNGKEYKCKVSNKALGAPIEKTISKAKGQPREPQVYTLPPCRDELTKNQVSLWCLVKGFYPSDIAVEWESNGQPENNYKTTPPVLDSDGSFFLYSKLTVDKSRWQQGNVFSCSVMHEALHNAYTQKSLSLSPGK

SEQ ID NO: 23 - protein, 463 aa, synthetic construct
  REGION 1..463: note = source = /note="Description of Artificial Sequence: Synthetic polypeptide"
QVQLVQSGAEVKKPGASVKVSCKASGYTFTGYYMHWVRQAPGQGLEWMGWINPNSGGTNYAQKFQGRVTMTRDTSISTAYMELSRLRSDDTAVYYCARSPNPYYYDSSGYYYPGAFDIWGQGTMVTVSSASVAAPSVFIFPPSDEQLKSGTASVVCLLNNFYPREAKVQWKVDNALQSGNSQESVTEQDSKDSTYSLSSTLTLSKADYEKHKVYACEVTHQGLSSPVTKSFNRGECDKTHTCPPCPAPEAAGGPSVFLFPPKPKDTLMASRTPEVTCVVVDVSHEDPEVKFNWYVDGVEVHNAKTKPREEQYNSTYRVVSVLTVLAQDWLNGKEYKCKVSNKALGAPIEKTISKAKGQPREPQVCTLPPSRDELTKNQVSLSCAVKGFYPSDIAVEWESNGQPENNYKTTPPVLDSDGSFFLVSKLTVDKSRWQQGNVFSCSVMHEALHNAYTQKSLSLSPGK

SEQ ID NO: 24 - protein, 214 aa, synthetic construct
  REGION 1..214: note = source = /note="Description of Artificial Sequence: Synthetic polypeptide"
DIQLTQSPSSLSASVGDRVTITCSASQDISNYLNWYQQKPGKAPKVLIYFTSSLHSGVPSRFSGSGSGTDFTLTISSLQPEDFATYYCQQYSTVPWTFGQGTKVEIKRTVAAPSVFIFPPSDEQLKSGTASVVCLLNNFYPREAKVQWKVDNALQSGNSQESVTEQDSKDSTYSLSSTLTLSKADYEKHKVYACEVTHQGLSSPVTKSFNRGEC

SEQ ID NO: 25 - protein, 213 aa, synthetic construct
  REGION 1..213: note = source = /note="Description of Artificial Sequence: Synthetic polypeptide"
SYVLTQPPSVSVAPGQTARITCGGNNIGSKSVHWYQQKPGQAPVLVVYDDSDRPSGIPERFSGSNSGNTATLTISRVEAGDEADYYCQVWDSSSDHWVFGGGTKLTVLSSASTKGPSVFPLAPSSKSTSGGTAALGCLVKDYFPEPVTVSWNSGALTSGVHTFPAVLQSSGLYSLSSVVTVPSSSLGTQTYICNVNHKPSNTKVDKKVEPKSC

SEQ ID NO: 26 - protein, 17 aa, synthetic construct
  REGION 1..17: note = source = /note="Description of Artificial Sequence: Synthetic peptide"
AIFTGSGAEYKAEWAKG

SEQ ID NO: 27 - protein, 13 aa, synthetic construct
  REGION 1..13: note = source = /note="Description of Artificial Sequence: Synthetic peptide"
DAGYDYPTHAMHY

SEQ ID NO: 28 - protein, 11 aa, synthetic construct
  REGION 1..11: note = source = /note="Description of Artificial Sequence: Synthetic peptide"
RASQGISSSLA

SEQ ID NO: 29 - protein, 7 aa, synthetic construct
  REGION 1..7: note = source = /note="Description of Artificial Sequence: Synthetic peptide"
GASETES

SEQ ID NO: 30 - protein, 12 aa, synthetic construct
  REGION 1..12: note = source = /note="Description of Artificial Sequence: Synthetic peptide"
QNTKVGSSYGNT

SEQ ID NO: 31 - protein, 123 aa, synthetic construct [sequence omitted]

SEQ ID NO: 32 - protein, 110 aa, synthetic construct [sequence omitted]

SEQ ID NO: 33 - DNA, 20 bp, synthetic construct
  misc_feature 1..20: note = source = /note="Description of Artificial Sequence: Synthetic oligonucleotide"
  source 1..20: mol_type = other DNA
gggtcgggggagcagctcgg

SEQ ID NO: 34 - DNA, 20 bp, synthetic construct
  misc_feature 1..20: note = source = /note="Description of Artificial Sequence: Synthetic oligonucleotide"
  source 1..20: mol_type = other DNA
tcatcacagtcctgcctagg

SEQ ID NO: 35 - DNA, 20 bp, synthetic construct
  misc_feature 1..20: note = source = /note="Description of Artificial Sequence: Synthetic oligonucleotide"
  source 1..20: mol_type = other DNA
atggcgtctggacaaggacc